(19) United States
(12) Patent Application Publication
(10) Pub. No.: US 2012/0141603 A1
(43) Pub. Date: Jun. 7, 2012

(54) METHODS AND COMPOSITIONS FOR LUNG CANCER PROGNOSIS

(75) Inventors: Ming-Sound Tsao, Toronto (CA); Kenneth Craddock, Toronto (CA); Wan Lam, Vancouver (CA); Timon Buys, Vancouver (CA); Igor Jurisica, Toronto (CA); Frances A. Shepherd, Toronto (CA)

(73) Assignees: BRITISH COLUMBIA CANCER AGENCY BRANCH, Vancouver, BC (CA); UNIVERSITY HEALTH NETWORK, Toronto, ON (CA)

(21) Appl. No.: 13/265,583

(22) PCT Filed: Apr. 21, 2010
(86) PCT No.: PCT/CA10/00619

§ 371 Date: Jan. 6, 2012

Related U.S. Application Data
(60) Provisional application No. 61/171,356, filed on Apr. 21, 2009.
(60) Provisional application No. 61/171,687, filed on Apr. 22, 2009.

(51) Int. Cl. C12Q 1/68; A61P 35/00; A61K 31/282; A61K 31/475; C40B 30/04; A61K 33/24

(52) U.S. Cl. 424/649; 435/6.12; 506/9; 514/492; 514/283

(57) ABSTRACT

Disclosed herein are methods and materials for prognosing survival of lung cancer patients, the methods comprising the detection of gains and losses of minimal common regions and/or genes associated with prognosis and benefit of chemotherapy.

This application claims the benefit of 35 U.S.C. 119 based on the priority of co-pending U.S. provisional patent applications 61/171,356, filed Apr. 21, 2009 and 61/171,687 filed Apr. 22, 2009, each of which are herein incorporated by reference in their entirety.

### FIELD OF THE DISCLOSURE

The disclosure relates to methods and compositions for prognosing and selecting treatment for lung cancer, particularly non-small cell lung carcinomas (NSCLC).

### BACKGROUND OF THE DISCLOSURE

Lung cancer is the leading cause of cancer death in Canada (Canadian Cancer Society, 2008). Even after complete surgical resection of stage I-III non-small-cell lung cancer (NSCLC), approximately half of patients will recur and die within 5 years (Azzoli et al, 2008). Current NSCLC clinicopathologic staging is not adequate to accurately predict which patients will be cured by surgery alone, and which patients with high risk of disease recurrence and mortality need adjuvant therapies.

Many studies have examined gene and protein expression patterns in NSCLC for refining the prognostication and treatment of the disease, with some success. However, the impact on patient survival and response to therapy for gene copy number alterations (amplifications and deletions) is an area that has not been well studied in this regard.

Gene copy number changes are worthy of close examination in NSCLC, because they have been shown to provide important information in other malignancies. HER2/neu amplification in breast cancer is the best-known example, where it has been shown to impart a much worse survival (Slamon D J et al, 1987) as well as predict the response to systemic chemotherapies (Dhesy-Thind et al, 2007). B-cell chronic lymphocytic leukemia/lymphoma is another well-studied example; deletions at 13q14 have been shown to be associated with prolonged survival, whereas deletions at 11q22-23 and at the TP53 locus on chromosome 17p have both been associated with a poor prognosis (Jaffe, 2003). Many similar discoveries of associations of gene copy number gains and losses with patient outcome are rapidly being discovered in many different malignancies. Detailed mechanistic studies may help further our understanding of the pathobiology and ultimately provide better treatments for patients.

Microarray comparative genomic hybridization (array-CGH) is a relatively new technique, capable of detecting gains and losses of genomic material at high-resolution across the genome, that has begun to revolutionize this body of knowledge. Recent studies have demonstrated the ability of array-CGH to subtype breast carcinomas (Climent et al, 2007a), DLBCL (Tagawa et al, 2005), CLL (Patel et al, 2008), and gliomas (Idbaih et al, 2008) into distinct groups based on their pattern of gains and losses. Many studies have shown an impact of specific gains or losses on patient survival, including colorectal carcinoma (Kim et al, 2006), gastric adenocarcinoma (Weiss et al, 2004), breast carcinoma (Han et al, 2006), mantle cell lymphoma (Rubio-Moscardo et al, 2005), diffuse large B cell lymphoma (Chen et al., 2006; Tagawa et al, 2005), neuroblastoma (Tomioka et al, 2008), and gliomas (Idbaih et al, 2008). In one study of breast carcinomas from patients enrolled in a clinical trial, the loss of a specific region of chromosome 11q was shown to be associated with a good response to anthracycline-based chemotherapy (Climent et al, 2007b). Two previous studies have shown that reliable array-CGH profiles can be obtained using archival formalin-fixed, paraffin-embedded (FFPE) tissues (Fenesterer et al, 2007; Mayr et al, 2006). This is very important, as it allows this powerful technique to be performed on the vast quantity of routinely handled and archived surgical specimens of diagnostic laboratories.

Similar to other epithelial malignancies, the karyotypes of NSCLC show multiple and complex chromosomal aberrations, resulting in net gain or loss of genetic material, indicative of genomic instability (Balsara et al, 2002). The imbalance profiles of the histologic subtypes of NSCLC (adenocarcinoma, squamous carcinoma, and large cell carcinoma) are similar, with frequent gains involving 5p, 8q, 3q, and 1q, frequent losses at 3p, 8p, 9p, 13q, and 17p, and often showing polyploidy (in the range of 58-102 chromosomes per cell) (Hoglund et al, 2004). Amplifications are commonly observed in the form of double minutes. Knowledge of the order or progression of these aberrations is scarce, but some have speculated that early events include trisomy 7, loss at 3p, and trisomy 12. Gains at both 7q and 8q have been associated with higher stage tumours with positive nodal involvement and higher tumour grade, and 20q13 gains have been linked with invasiveness in adenocarcinoma (AC).

Genes reported to be amplified have included MYC, TERT, cyclin D1, and EGFR. Increased epidermal growth factor (EGFR) copy number are seen in 8-30% of patients by FISH and qPCR, and are often seen in conjunction with mutations in the EGFR tyrosine kinase domain (Thomas et al, 2006). Both amplification and mutations are associated with a specific demographic: East Asian, female, never smokers, with adenocarcinomas often showing a bronchioloalveolar histologic pattern (Sequist et al, 2007). Studies have shown that these patients tend to have a rapid, dramatic, and durable response to gefitinib, a drug specifically designed to inhibit the tyrosine kinase signaling activity of EGFR (Cappuzo et al, 2005; Takano et al, 2005; Hirsch et al, 2007). This finding is an exciting example of how the identification of genetic events such as amplification and mutation can lead to effective targeted therapies. Strategies such as this could eventually lead to effective individualized chemotherapy designed against many other altered pathways.

P63 amplification has also been shown to have a prognostic utility in NSCLC. Massion et al (2003) applied FISH and immunohistochemistry to detect P63 gene amplification and protein expression in tissue microarrays containing 217 NSCLC samples. They found that P63 copy number >=3 and increased immunostaining intensity were both significantly associated with a better survival.

Array-CGH has allowed researchers to study gene copy number aberrations in even greater detail (Dehan et al, 2007; Choi et al, 2007; Zhao et al, 2005; Jiang et al, 2004). The high resolution of this technique is clarifying genomic amplification and deletion to regions often containing only a few genes, as well as identifying small, previously undetected aberrations. As a result, the list of genes implicated in NSCLC pathobiology is growing rapidly. Tonon et al. (2005) identified 93 minimal common regions (MCRs) of aberration in NSCLC tumours and cell lines, 21 of which spanned less than 0.5 Mb with a median of 5 genes in each, with virtually all genes previously implicated in NSCLC pathogenesis present within these regions, as well as many novel candidate genes. Patterns of aberrations were similar between adenocarcinoma (AC) and squamous carcinoma (SqC); supervised or unsupervised clustering was unable to differentiate the two. Only the amplification on 3q26-29 has been targeted significantly in SqC, similar to previous findings by Massion et al (2002).

In a large study of 371 adenocarcinomas using SNP array-CGH, Weir et al (2007) identified 26 recurrent large-scale events involving gain or loss of at least half of a chromosome, together comprising more than half of the human genome. In addition, 31 focal amplifications and homozygous deletions were identified, including multiple novel candidate genes. One of the homozygously deleted genes was PTPRD, a tyrosine phosphatase. Upon sequencing of this gene, somatic mutations were found in 11 of 188 samples, indicating a role in PTPRD dysregulation in a subset of ACs. The most common focal amplification, at 14q13.3, contained no known proto-oncogene. Biological studies using RNAi knockout of the 2 genes found within this region identified that NKX2-1 as a key factor in the growth of cell lines with 14q amplifications.

Findings such as these highlight the power and utility of array-CGH for finding specific molecular aberrations in subsets of NSCLC. However, lacking in the literature are studies correlating these genomic events with patient outcome. Shibata et al (2005) studied 55 ACs and were able to split the tumours into 3 groups by unsupervised hierarchical clustering. These clusters were associated with distinct genetic alterations and showed an association with smoking history and gender, but no association with stage or disease-free survival. However, two specific alterations did show an association with disease-free survival on multivariate analysis: loss on 13q14.1 and gain of 8q24.2 were both associated with a poor outcome.

Materials and methods for prognosing lung cancer and selecting effective treatment for subjects with lung cancer, particularly non-small cell lung carcinomas (NSCLC) would be useful.

### SUMMARY OF THE DISCLOSURE

Disclosed herein are genes and genomic regions, the gain or loss of which are associated with prognosis of lung cancer. A subset are associated with significant improvement when administered chemotherapy. Detecting the gains and losses are useful for determining a prognosis for a subject with lung cancer and for guiding treatment selection.

Accordingly in an aspect, the disclosure provides a method for determining a lung cancer prognosis in a subject, the method comprising: (a) determining a genomic profile comprising detecting the presence or absence of one or more genomic alterations in one or more of chromosomes 2, 11, 4, 5, 7, 9, 12, 17, 19, 20, 8, 1, 13, 16, 6 and/or 14 listed in Tables 1-11 in a biological sample from the subject; wherein the prognosis is determined to be poor when the genomic profile comprises a gain of all or part of one or more minimal common regions (MCRs) and/or genes within one or more of chromosomes 1, 2, 11, 4, 5, 6, 7, 9, 12, 14, 16, 17, 19 and/or 20, listed as associated with poor prognosis (e.g. associated with survival) in Tables 1, 2, 5, 9, 10, and/or 11 and/or a loss of all or part of one or more MCRs and/or genes within one or more chromosomes 1, 5, 8, 13 and/or 16 listed as associated with poor prognosis in Table 3 and/or 7 and the prognosis is determined to be good when the genomic profile comprises a genomic gain of all or part of a MCR and/or gne within chromosome 8 listed as associated with good prognosis in Table 6 and/or a loss of all or part of one or more MCRs and/or genes within chromosome 2, 6, 9 and/or 14 listed as associated with good prognosis in Tables 8 relative to a control.

In an embodiment, the method comprises: (a) determining a genomic profile comprising detecting the presence or absence all or part of one or more genomic alterations in one or more of chromosomes 2, 11, 4, 5, 7, 9, 12, 17, 19, 20, 8, 1, 13, 16, 6 and/or 14 and/or genes listed in Tables 1-11 in a biological sample from the subject; (b) determining the lung cancer prognosis for the subject by comparing the genomic profile with one or more controls, wherein the prognosis is determined to be poor when the genomic profile comprises a gain of all or part of one or more minimal common regions (MCRs) and/or genes within chromosomes 1, 2, 11, 4, 5, 6, 7, 9, 12, 14, 16, 17, 19 and/or 20, listed as associated with poor prognosis in Tables 1, 2, 5, 9, 10, and/or 11 and/or a loss of all or part of one or more MCRs within chromosomes 1, 5, 8, 13 and/or 16 listed as associated with poor prognosis in Tables 3 and/or 7; and the prognosis is determined to be good when the genomic profile comprises a genomic gain of all or part of a MCR and/or gene within chromosome 8 listed as associated with good prognosis in Table 6 and/or a loss of all or part of one or more MCRs and/or genes within chromosome 2, 6, 9 and/or 14 listed as associated with good prognosis in Table 6 and/or 8 relative to the control.

In an embodiment, the method comprises obtaining a biological sample for determining the genomic profile.

In an embodiment, the prognosis is determined to be poor when the genomic profile comprises a gain of all or part of a gene listed in Table 5, and/or comprises a loss of all or part of a gene listed in Table 7, and the prognosis is determined to be good when the genomic profile comprises a gain of all or part of a gene listed in Table 6 and/or a loss of all or part of a gene listed in Table 8 relative to the control. In an embodiment, the prognosis is determined to be poor when the genomic profile comprises a gain of all or part of a gene listed in Table 9, and/or 11 identified as associated significantly and/or trending to significance with poor prognosis. In an embodiment, the gene associated with prognosis is a gene that shows a trend to significance. In another embodiment, the gene associated with prognosis is a gene with a significant association.

In an embodiment, the presence or absence of a genomic alteration is determined using a chromosomal probe and detecting a hybridization pattern.

In another embodiment, the prognosis is determined to be poor when the hybridization pattern indicates a gain of all or part of a MCR or a gene listed in Table 1, 2, 5 and/or 9-11 (for genes identified as associated with poor prognosis) and/or loss of all or part a MCR or gene listed in Table 3 and/or 7. In a further embodiment, the gain comprises all or part of a gene listed in Table 5 and/or the loss comprises all or part of a gene listed in Table 7. In yet another embodiment, the gain comprises all or part of one or more of genes listed in Tables 9 and/or 11.

In another embodiment, the method comprises detection of a gain of all or part of one or more of the genes listed in Table 9 and/or 11 for genes identified as associated significantly with poor prognosis (and/or trending to poor prognosis) including ANGPT1, HOXC11, ITGA7, PRIM1, B4GALNT1, OS9, CDK4, and/or TSFM (e.g. Table 9 genes) and/or GUCA2A, LEPRE1, C1orf50, FGF3, FAM112B, B4GALNT1, OS9, CENTG1, CDK4, TSFM, AK024870, NUP107, MDM2, CPSF6, BCL11B, ASXH1 and/or C20orf112 (e.g. Table 11 genes).

In another embodiment, the prognosis is determined to be good when the hybridization pattern indicates a gain of all or part of a MCR within chromosome 8 associated with good prognosis and/or a loss of all or part of one or more MCRs within chromosome 6 or 14 associated with good prognosis relative to a control. In an embodiment, the gain comprises all or part of RAB11FIP1 and/or the loss comprises all or part of a gene listed in Table 8.

In an embodiment, the presence or absence of a genomic alteration is determined using a chromosomal probe. In another embodiment, the control is a control copy number, centromere copy number or a control gene on the same or different chromosome.

In another aspect, the disclosure includes a method for determining a likelihood of improved survival or response with chemotherapy treatment comprising detecting a gain of all or part of a MCR or gene listed in Tables 1, 2, 5, 9, 10 and/or 11 associated with improved response to chemotherapy, wherein a gain indicates the subject has a good prognosis when treated with chemotherapy relative to a subject not treated with chemotherapy.

In another aspect, the disclosure includes a method for determining tumour responsiveness to a chemotherapy treatment comprising detecting a gain of all or part of one or more of the genes listed in Tables 1, 2, 5, 9 or 11 associated with improved response to chemotherapy, wherein a gain indicates the tumour is likely responsive to treatment with chemotherapy relative to a tumour not comprising the gain.

In an embodiment, the gain associated with improved survival with chemotherapy or improved tumor responsiveness is a gain of all or part of one or more of the following genes: MFSD7, D4S234E, ACOX3, SRD5A1, AQP2, ACCN2, SLC11A2, SCN8A, KRT81, KRT1, ESPL1, NPFF, ATP5G2, HOXC11, NEUROD4, ZBTB39, KIAA0286, INHBE, MARS, B4GALNT1, TSFM, DNMT3B.

In another embodiment, the gain associated with improved survival with chemotherapy or improved tumor responsiveness is a gain of all or part of one or more of the following genes: BAALC, ANGPT1, MYC, WISP1, KRT81, KRT1, NEUROD4, and/or PA2G4 (e.g. Table 9 genes associated with improved response to chemotherapy). In a further embodiment, the gain associated with improved survival with chemotherapy or improved tumor responsiveness is a gain of all or part of one or more of the following genes: GUCA2A, PPIH, LEPRE1, CR623026, C1orf50, DQ515898, DQ515897, MYC FGF3, KRT81, KRT1, FAM112B, B4GALNT1, CENTG1, BCL11B (e.g. Table 11 genes associated with improved response to chemotherapy).

In another aspect, the disclosure includes a method for determining a likelihood of improved survival with chemotherapy treatment comprising detecting a loss of all or part of a MCR and/or gene listed in Tables 3, 4, 7 and/or 8 associated with improved response to chemotherapy, wherein the loss indicates the subject has a good prognosis when treated with chemotherapy relative to a subject not treated with chemotherapy.

In another aspect, the disclosure includes a method for determining tumour responsiveness to a chemotherapy treatment comprising detecting a loss of all or part of a MCR and/or gene listed in Tables 3, 4, 7 and/or 8 associated with improved response to chemotherapy, wherein the loss indicates the tumour is likely responsive to treatment with chemotherapy relative to a tumour not comprising the loss.

In an embodiment, the loss is of all or part of one of the following genes: RHOC, ATP2C2, ZDHHC7, COC4I1, and/or FOXF1.

In another embodiment, the lung cancer is non-small cell lung cancer (NSCLC), early stage NSCLC, squamous cell carcinoma or adenocarcinoma and/or metastatic lung cancer.

In another aspect, the method further comprises detecting the expression level of a gene listed in Table 5, 6, 7, 8, 9 and/or 11. For example, the expression level of a gene associated with prognosis and/or response to chemotherapy can be detected for predicting a prognosis and/or for predicting tumour responsiveness. In an embodiment, the expression level of the gene all or partly gained or lost is increased or decreased respectively, relative to a control expression level wherein increased expression of a gene gain listed in Table 5 and/or decreased expression of a gene listed in Table 7 indicates poor prognosis without chemotherapy, and/or increased expression of a gene listed in Table 6 and/or decreased expression of a gene listed in Table 8 indicates good prognosis. In a further embodiment, the expression level of a gene listed in Table 9 or 11 is detected.

Another aspect comprises a method for determining a lung cancer prognosis in a subject, the method comprising: (a) determining a hybridization pattern of a chromosomal probe or a set of chromosomal probes in a biological sample from the subject, wherein the probe or probeset is targeted to all or part of one or more MCRs listed in the provided tables, including but not limited to NRG4 on the short arm of chromosome 1 (1p), NRG58 on 8q, NRG74 on 11q, NRG79 on 12q, NRG80 on 12q, NRG81 on 12q, NRG82 on 12q, and/or NRG89 on 14q; (b) determining the prognosis and/or predicting the response to chemotherapy for a patient with lung cancer based on the hybridization pattern, wherein the prognosis is determined to be poor without chemotherapy when the hybridization pattern indicates a gain of DNA copy number at an MCR on 11q and/or a gain at an MCR on 12q and/or a gain at an MCR on 14q relative to a control; and/or the prognosis is determined to be good when treated with chemotherapy when the hybridization pattern indicates a gain of DNA copy number within an MCR on 1p and/or 8q and/or 11q and/or 12q and/or 14q.

In an embodiment, the gain of DNA copy number is at or within an MCR located at approximately base-pair positions 41265460 to about 43221579 on the short arm of chromosome 1, and is indicative of a good prognosis with chemotherapy.

In another embodiment, the gain of DNA copy number is at an MCR located at approximately base-pair positions 128289292 to about 128936748 on the long arm of chromosome 8 is indicative of a good prognosis with chemotherapy.

In another embodiment, the gain of DNA copy number is at or within an MCR located at approximately base-pair positions 68572940 to about 70388868 on the long arm of chromosome 11 is indicative of a good prognosis with chemotherapy.

In another embodiment, the gain of DNA copy number is at or within an MCR located at approximately base-pair positions 50731457 to about 51457372 on the long arm of chromosome 12 is indicative of a good prognosis with chemotherapy.

In another embodiment, the gain of DNA copy number is at or within an MCR located at approximately base-pair positions 52696908 to about 53538441 on the long arm of chromosome 12 is indicative of a good prognosis with chemotherapy.

In another embodiment, the gain of DNA copy number is at or within an MCR located at approximately base-pair positions 55933813 to about 57461765 on the long arm of chromosome 12 is indicative of a good prognosis with chemotherapy.

In another embodiment, the gain of DNA copy number is at or within an MCR located at approximately base-pair positions 96994959 to about 99058653 on the long arm of chromosome 14 is indicative of a good prognosis with chemotherapy.

Another aspect relates to a method of selecting a treatment regimen for a subject with lung cancer, the method comprising: (a) determining a genomic profile comprising detecting a genomic alteration of all or part of one or more MCRs and/or genes selected from MCRs and genes identified herein associated with survival with chemotherapy, for example as listed in Table 1, 2, 3, 5, 7, 9, 10 and/or 11; in a biological sample from the subject; and (b) selecting chemotherapy when a gain or loss associated with improved survival with chemotherapy is detected and/or not selecting chemotherapy and/or selecting a non-chemotherapy and/or a non-platinum analog-, a vinca alkyloid- and/or combination thereof chemotherapy, when a gene associated with worse survival with chemotherapy.

In an embodiment, the method comprises: (a) determining a genomic profile comprising detecting a genomic alteration in one or more genes selected from Table 5 and/or 7 in a biological sample from the subject; (b) selecting chemotherapy for the subject when the genomic profile comprises a gain of all or part of one or more of the following genes: MFSD7, D4S234E, ACOX3, SRD5A1, AQP2, ACCN2, SLC11A2, SCN8A, KRT81, KRT1, ESPL1, NPFF, ATP5G2, HOXC11, NEUROD4, ZBTB39, KIAA0286, INHBE, MARS, B4GALNT1, TSFM, and/or DNMT3B; and/or a loss of all or part of one or more of the following genes: RHOC, ATP2C2, ZDHHC7, COC4I1, and/or FOXF1 relative to a control.

In another embodiment, the method comprises: (a) determining a genomic profile comprising detecting a genomic alteration in one or more genes selected from Table 9 and/or 11 in a biological sample from the subject; (b) selecting chemotherapy for the subject when the genomic profile comprises a gain of all or part of one or more of the following genes: BAALC, ANGPT1, MYC, WISP1, KRT81, KRT1, NEUROD4, and/or PA2G4 (e.g. Table 9 genes associated with improved response to chemotherapy). In a further embodiment, the gain associated with improved survival with chemotherapy or improved tumor responsiveness is a gain of all or part of one or more of the following genes: GUCA2A, PPIH, LEPRE1, CR623026, C1orf50, DQ515898, DQ515897, MYC, FGF3, KRT81, KRT1, FAM112B, B4GALNT1, CENTG1, and/or BCL11B (e.g. Table 11 genes associated with improved response to chemotherapy). In an embodiment, the method comprises not selecting chemotherapy and/or not selecting a chemotherapeutic regimen comprising a platinum analog, a vinca alkyloid and/or a combination thereof e.g. selecting a non-chemotherapy and/or a non-platinum analog-, vinca alkyloid- or combination thereof chemotherapy, when a gain at AK024870, CPSF6 is detected.

In certain embodiments, the biological sample is selected from the group consisting of lung tissue, lung cells, lung biopsy and sputum, including formalin fixed, paraffin embedded and fresh frozen specimens.

Also provided is a method for determining a lung cancer prognosis in a subject, the method comprising: detecting the presence or absence of a genomic alteration at a locus identified in Tables 1, 2, 3, 4, 5, 6, 7, 8, 9, 10 and/or 11 in a biological sample from the subject, wherein the prognosis is determined to be poor in the absence of chemotherapy when a gain of all or part of a MCR listed in Tables 1 and/or 2 or a gene listed in Table 5 and/or a loss of all or part of a MCR listed in Table 3 and/or gene listed in Table 7 is detected; and the prognosis is determined to be good when a gain of all or part of a MCR or gene listed in Table 6 and/or loss of all or part of a MCR or gene in Table 4 and/or 8 is detected relative to a control. In another embodiment, a gain of all or part of a MCR listed in Table 10 and/or a gene listed in Table 9 and/or 11, wherein the prognosis is determined to be poor in the absence of chemotherapy when a gain associated with poor prognosis (including trending to poor prognosis) is detected.

In an embodiment, the presence or absence of a gain of DNA copy number is detected at an MCR at 12q at or within basepair positions 50731457 to 51457372, and/or 12q at or within basepair positions 52696908 to 53538441, and/or 12q at or within basepair positions 55933813 to 57461765, and/or 12q at or within basepair positions 64438067 to 68503251, and/or 14q at or within basepair positions 96994959 to 99058653. In another embodiment, the presence or absence of a gain of DNA copy number is detected at an MCR at 12q at or within basepair positions 50731457 to 51457372. In another embodiment, the presence or absence of a gain of DNA copy number is detected at an MCR at 12q at or within basepair positions 52696908 to 53538441. In another embodiment, the presence or absence of a gain of DNA copy number is detected at an MCR at 12q at or within basepair positions 55933813 to 57461765. In another embodiment, the presence or absence of a gain of DNA copy number is detected at an MCR at 12q at or within basepair positions 64438067 to 68503251. In another embodiment, the presence or absence of a gain of DNA copy number is detected at an MCR at 14q at or within basepair positions 96994959 to 99058653. In yet a further embodiment the genomic alteration comprises all or part of a MCR listed in Table 1, 2, 3, 4 and/or 10.

In an embodiment, the presence or absence of a DNA copy number alteration at for example, the position of a gene located within the MCRs gained or lost, for example genes within the MCRs listed in any one of Tables 1 to 11 are detected. In an embodiment, the presence or absence of a DNA copy number alteration at the position of a gene from the group consisting of KRT1, ESPL1, NPFF, ATP5G2, HOXC11, and/or genes within an MCR located between 50-57 Mb on chromosome arm 12q (e.g. MCR IDs NRG79, NRG80, NRG81, NRG82) is detected. In another embodiment, the presence or absence of a gene from the group consisting of ITGA7, CDK2, BCDO2, ERBB3, DLST, PA2G4, ZBTB39 and/or TSFM which are comprised in the MCRs at 55.2-55.6 Mbp on chromosome arm 12q are detected. In another embodiment, the gene detected is all or part of a gene listed in Table 9 and/or 11.

Another aspect provides a method of predicting response to a chemotherapeutic treatment in a subject with lung cancer comprising detecting the presence or absence of a gain or loss of all or part of a MCR or a gene in any one of Tables 1-11, predicting the response to the chemotherapeutic according to the presence or absence of the MCR or gene gain or loss compared to a control, wherein detecting a MCR or gene associated with improvement with chemotherapy predicts chemotherapy will be efficacious, for example will, improve survival and/or wherein detecting a MCR and/or gene not associated with improvement with chemotherapy predicts no response to chemotherapy.

A further aspect provides a method of determining a likelihood of improved survival in a lung cancer subject who was or is receiving a chemotherapeutic treatment, comprising determining the presence or absence of a gain or loss of all or part of a MCR and/or gene associated with improvement with chemotherapy, predicting the likelihood of improved survival according to the presence or absence of the MCR and/or gene gain or loss compared to a control, wherein detecting all or part of a gain and/or loss of a MCR and/or gene associated with improvement with chemotherapy predicts likelihood of improved survival compared to a control having the same gain and/or loss who has not received and/or is not receiving chemotherapy. In an embodiment, the presence of a gain and/or loss associated with improvement with chemotherapy is indicative of a favourable predisposition of the subject to respond to platinum analogs, vinca alkyloids and/or a combination thereof.

In certain embodiments, the genomic alteration, MCR and/or gene gain or loss is determined by array CGH, FISH, chromagen in situ hybridization (CISH) or PCR.

Another aspect provides a method of treating lung cancer comprising determining the presence or absence of a gain and/or loss of all or part of a MCR and/or gene associated with improvement with chemotherapy in a subject with lung cancer and administering chemotherapy to the subject with at least one gain or loss associated with improvement with chemotherapy.

In an embodiment, the chemotherapy is a platinum analog, a vinca alkaloid or a combination thereof. In a further embodiment, the platinum analog is selected from the group consisting of cisplatin, paraplatin, carboplatin, oxaliplatin and satraplatin in either IV or oral form. In another embodiment, the vinca alkaloid is selected from the group vinorelbine, vincristine, vinblastine, vindesine and vinflunine in either IV or oral form.

A further aspect relates to a composition comprising a detection agent for detecting all or part of a MCR and/or gene gain or loss associated with prognosis. In an embodiment, the composition comprises a probe that binds and/or hybridizes with all or part of a MCR and/or a gene described herein, and/or a primer or primer pair for amplifying a polynucleotide comprising all or part of a MCR and/or gene associated with prognosis described herein. In an embodiment, the probe is a BAC clone listed in Table 13 and/or the primer is a primer listed in Table 12.

Yet a further aspect provided is a kit for determining lung cancer prognosis in a subject. In and embodiment, the kit comprises a chromosomal probe and/or a set of chromosomal probes, wherein the probe or set comprises a probe to a MCR or part thereof listed in any one of Tables 1 to 11 and/or a gene or part thereof listed in Tables 5, 6, 7, 8, 9 and/or 11. In another embodiment the kit comprises one or more gene expression probes, wherein a probe is specific for a gene expression product of a gene listed in Tables 5, 6, 7, 8, 9 and/or 11. In an embodiment, the probes are labeled, optionally fluorescently labeled or labelled with a chromagen. In another embodiment, the probes are comprised in an array on a solid support. In yet a further embodiment, the kit further comprising instructions that indicate prognosis is determined to be poor when a hybridization pattern of the set of chromosomal probes indicates a gain in all or part of a MCR in 12q, and/or a gain in all or part of a MCR comprising all or part of a gene listed in Table 5, 9 and/or 11 and/or a loss of all or part of a MCR comprising all or part of a gene listed in Table 7, relative to control; and/or to be good when a hybridization pattern of chromosomal probes indicates a gain in all or part of a MCR comprising all or part of a gene listed in Table 6 and/or a loss of all or part of a MCR comprising all or part of a gene listed in Table 8; optionally wherein the control is centromere copy number.

In an embodiment, the kit comprises a reagent for FISH analysis of a MCR or a gene gain or loss described herein, for example, the kit comprises a probe for a MCR or gene gain or loss described herein, for example a BAC clone comprising all or part of a target MCR or gene, including for example the BAC clones listed in Table 13 and/or labeling reagents for labeling the probe. In a further embodiment, the kit comprises a reagent for CGH analysis of a MCR or gene gain or loss described herein, for example, the kit comprises an array with one or more probes for detecting all or part of one or more MCRs or genes gained or lost described herein and/or labeling reagents for labeling the subject sample DNA. In a further embodiment, the kit comprises a reagent for PCR such as quantitative or multiplex PCR, for example the kit comprises a primer set for amplifying all or part of a MCR or gene described herein associated with prognosis.

Unless otherwise defined, all technical and scientific terms used herein have the same meaning as commonly understood by one of ordinary skill in the art to which this disclosure pertains. Although methods and materials similar or equivalent to those described herein can be used in the practice or testing of methods and compositions described herein, a few selected suitable methods and materials are described in more details below. All publications, patent applications, patents, and other references mentioned herein are incorporated by reference in their entirety, including nucleic acid sequences identified by Entrez Gene ID, unigene ID or other gene identifier number referred to herein and particularly as provided in the Tables. In case of conflict, the present specification, including definitions, will control. In addition, the materials, methods, and examples are illustrative only and are not intended to be limiting in any respect.

All embodiments of the disclosure, including those described under different aspects of the disclosure, are contemplated to be combined with other embodiments whenever applicable.

Other features and advantages of the present disclosure will become apparent from the following detailed description and claims. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### DETAILED DESCRIPTION OF THE DISCLOSURE

### I. Definitions

The term “lung cancer” as used herein refers to cancers of the tissues or cells of the lung including for example non-small cell lung cancer (NSCLC), and small cell lung cancer (SCLC). The term could also be used to refer to cancers that have arisen in the lung and have metastasized to other sites (e.g. brain, liver, adrenals).

The term “non-small cell lung cancer” as used herein refers to primary lung cancer that is distinguished from small cell lung cancer and that is composed of multiple different types, including adenocarcinoma, squamous cell carcinoma, large cell carcinoma and other less frequent types.

The term “lung adenocarcinoma” and/or “lung ADC” and/or “pulmonary ADC” as used herein refer to a type of lung cancer and comprises various subtypes including bronchioloalveolar carcinoma (BAC) which is non invasive and/or includes focal invasion and has good prognosis (2) and invasive ADC including mixed type, which can have areas with BAC like pattern and is referred to as invasive ADC with BAC features (AWBF).

The term “control” as used herein refers to a specific value or dataset e.g., control expression level, control gene copy number, reference expression profile or reference genomic profile according to the context which a person skilled in the art would readily understand, derived from one or more samples of a known subject class e.g., lung cancer free class not having a MCR or a gene gain or loss described herein, that is suitable for comparison to the value or dataset derived from a subject sample. For example, the control can be a value or dataset derived from tumor adjacent non-neoplastic normal tissue or tissue from a disease free subject, e.g. for comparing to a lung cancer subject gene expression profile. With respect to genomic alterations e.g. gains and losses, the control can for example also refer to an internal control e.g. the copy number of a non-altered region of the chromosome or a different chromosome e.g. a chromosome with minimal variance in lung cancer subjects, for example a chromosome not herein or previously identified as associated with prognosis. Such methods wherein an internal control is useful include for example quantitative polymerase chain reaction (PCR) or fluorescent in situ hybridization (FISH). Optionally, the copy number can be compared to the centromere for example when using FISH. Typically a normal or control genomic profile refers to a single genomic copy on each of the two alleles. For example in the array-CGH, the control is a normal reference genomic DNA that is assumed to have 2 copies of each gene. In other examples, a positive control is employed, for example, a sample or standard corresponding to subject comprising the gain or loss associated with prognosis and/or response to chemotherapy, useful for example for quantitative PCR and/or FISH methods, for example included in quantitative PCR and/or FISH based kits. Based on the teachings herein and knowledge in the field, a person skilled in the art would readily be able to identify suitable controls for the methods described herein.

The term “disease free subject” refers to a subject that is free of lung cancer.

The term “microarray” as used herein, refers to an array of distinct polynucleotides or oligonucleotides synthesized or spotted (e.g. in the case of BAC clones) on a substrate, such as paper, nylon or other type of membrane, filter, chip, glass slide, or any other suitable solid support.

The terms “complementary” or “complementarity”, as used herein, refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some nucleotides or portions of the nucleotide sequences of the nucleic acids bind, or it may be complete when total complementarity exists between the single stranded molecules. The degree of complementarity between nucleic acid strands has significant effects on the efficiency and strength of hybridization between nucleic acid strands.

“Amplification of polynucleotides” can be achieved by utilization of s methods such as the polymerase chain reaction (PCR), including for example quantitative PCR, multiplex PCR and multiplex ligation dependent probe amplification (MLPA), ligation amplification (or ligase chain reaction, LCR) and amplification methods based on the use of Q-beta replicase. These methods are well known and widely practiced in the art. Reagents and hardware for conducting PCR are commercially available. Primers useful to amplify specific sequences from selected genomic regions are preferably complementary to, and hybridize specifically to sequences flanking the target genomic regions.

The term “reference profile” as used herein refers to a reference expression profile, a reference genomic profile, and/or a reference gene copy number profile according to the context.

A “reference expression profile” as used herein refers to the expression signature of a subset of biomarkers (e.g. one or more), which correspond to genes associated with a prognosis class e.g. poor prognosis or good prognosis +/− chemotherapy and/or a control.

The term “expression level” as used herein refers to the absolute or relative amount of the transcription and/or translation product of a gene described herein and includes RNA and polypeptide products.

A “reference gene copy number profile” as used herein refers to the gene copy number of a subset of genes (e.g one or more) listed in Tables 5, 6, 7, 8, 9 and/or 11. The reference gene copy number profile is optionally a reference number, typically 2, and/or identified using for example using normal human tissue and/or cells and/or tissue and/or cells from lung cancer. Normal tissue and/or cells includes for example, tumor adjacent non-neoplastic tissue and/or cells and/or tissue and/or cells from a lung cancer disease free subject. The reference gene copy number profile is accordingly a reference signature of the copy number of a subset of genes in Tables 5, 6, 7, 8, 9 and/or 11, to which the subject gene copy number of the corresponding genes in a sample of a subject are compared.

The term “genomic profile” as used herein refers to the genomic structural signature of a subject genome. A number of variations and alterations referred to as copy number variations, have been characterized including amplifications and deletions, a subset of which are associated with disease. The alterations can comprise small and large amplifications and/or deletions which can occur through out the genome.

The phrase “determining a genomic profile” as used herein refers to detecting the presence, absence, frequency, variability and/or length of one or more genomic alterations including amplifications and deletions of all or part of one or more MCRs and/or which may or may not comprise alterations in the coding nucleic acid sequence of genes e.g., can comprise alterations in the intergenic regions of the genome, such as those found for example on 12q, 8q and 11q. Genomic alterations comprising amplifications and deletions in all or part of one or more genes comprise those listed in Tables 5, 6, 7, 8, 9 and/or 11. A person skilled in the art will appreciate that a number of methods can be used to determine a genomic profile, including for example fluorescence and other non-fluorescent types of in situ hybridization (FISH, CISH or others), and quantitative PCR (qPCR), multiplex PCR including for example multiplex ligation dependent probe amplification (MLPA) and array CGH.

The term “reference genomic profile” as used herein refers a genomic signature comprising genomic alterations, associated with prognosis with or without chemotherapy. The reference genomic profile is optionally a normal reference genomic DNA (e.g. a control) that is assumed to have 2 copies of each gene and/or is derived from normal human tissues and/or cells. The reference genomic profile is accordingly for example, normal genomic copy number to which a subject genomic profile is compared for classifying the tumor or determining or predicting clinical outcome.

The term “chemotherapy” as used herein means treatment with anticancer drugs, including but not limited to treatment with vinca alkaloids for example vinorelbine vinblastine, vincristine, vinflunine and/or vindesine in for example IV or oral form and/or platinum analogues for example cisplatin, carboplatin, paraplatin, satraplatin and/or oxaliplatin in for example IV or oral form.

The term “chemotherapeutic” as used herein means an anticancer drug, including but not limited to treatment with mitotic inhibitors such as vinca alkaloids for example vinorelbine vinblastine, vincristine, and/or vindesine or analogs thereof and/or DNA alkylating agents such as platinum based chemotherapeutics for example cisplatin, carboplatin and oxaliplatin.

The term “similar” or “similarity” as used herein with respect to a reference profile refers to similarly in both the identity and quantum of change in expression level of a biomarker, genomic alteration, or gene copy number variation compared to a control where the control is for example derived from a normal cell and/or tissue or has a known outcome class such as poor survival or good survival.

The term “similarity in expression” as used herein means that there is no or little difference, for example no statistical difference, in the level of expression of the biomarkers between the test sample and the control and/or between good and poor prognosis groups defined by biomarker expression levels.

The term “most similar” in the context of a reference profile refers to a reference profile that is associated with a clinical outcome that shows the greatest number of identities and/or degree of changes with the subject profile.

The term “differentially expressed” or “differential expression” as used herein refers to biomarkers described herein that are expressed at one level in a prognostic group and expressed at another level in a control. The differential expression can be assayed by measuring the level of expression of the transcription and/or translation products of the biomarkers, such as the difference in level of messenger RNA transcript expressed or polypeptide expressed in a test sample and a control. The difference can be statistically significant.

The term “difference in the level of expression” refers to an increase or decrease in the measurable expression level of a given biomarker expression product as measured by the amount of messenger RNA transcript and/or the amount of polypeptide in a sample as compared with the measurable expression level of a given biomarker in a control. In one embodiment, the differential expression can be compared using the ratio of the level of expression of a given biomarker or biomarkers as compared with the expression level of the given biomarker or biomarkers of a control, wherein the ratio is not equal to 1.0. For example, an RNA or polypeptide is differentially expressed if the ratio of the level of expression in a first sample as compared with a second sample is greater than or less than 1.0. For example, a ratio of greater than 1.1, 1.2, 1.5, 1.7, 2, 3, 3, 5, 10, 15, 20 or more, or a ratio less than 0.9, 0.8, 0.6, 0.4, 0.2, 0.1, 0.05, 0.001 or less. A sample can be compared to a group to identify differential expression. For example, one could compare a sample of interest to a group of control samples and use a p-value to demonstrate statistically that the sample of interest is for example overexpressing the RNA product of a gene or has an increased DNA copy number at that gene compared to control samples.

The term “prognosis” as used herein refers to a clinical outcome e.g. a poor survival or a good survival, and includes for example survival outcome in the absence of chemotherapy and/or improved survival with administration of chemotherapy. Good prognosis and improved survival are used herein interchangeably as are poor prognosis and poor survival. As demonstrated herein, prognosis is associated with the presence or absence of a gain or loss of specific MCRs and genes described herein, compared to a reference profile such as a reference expression profile, or a reference gene copy number profile of a suitable comparator group. For example, subjects with gains in MCRs and/or genes listed in for example Tables 1, 2, 5, 9, 10 and/or 11 or loss of MCRs and/or genes in Table 3, 4, and/or 7 have a poor prognosis or poor survival compared to subjects not having these gains or losses for regions identified. Accordingly, the prognosis provides an indication of disease progression and includes an indication of likelihood of recurrence, metastasis, death due to disease e.g. survival, tumor subtype or tumor type.

The term “associated with a prognosis” as used herein refers to gains and/or losses in all or part of a MCR and/or gene associated with survival identified in the Tables as associated with for example, poor survival in the absence of chemotherapy and/or listed in the Tables as associated with improved survival with chemotherapy, as well as for example MCRs and/or genes listed in the Tables as associated with good and/or prognosis. The term “associated with a poor prognosis” identifies the subset shown to statistically or trend to poor survival with surgery alone e.g. in the absence of chemotherapy (and/or the presence of chemotherapy for gains at AK024870 and/or CPSF6).

The term “tumour responsiveness” as used herein refers to the likelihood that a subject's lung cancer will or will not respond to chemotherapy treatment. It has been determined that a subset gains or losses associated with prognosis are associated with benefit from chemotherapy such that a subject with these gains or losses have an improved survival when treated with chemotherapy compared to a subject not receiving chemotherapy with the same gain or loss. Gains have also been associated with worse survival. For example, a gain or increased expression of ANK024870 and/or CPSF6 is associated with worse survival with administration of chemotherapy.

The term “classifying” as used herein refers to assigning, to a class or kind, an unclassified item. A “class” or “group” then being a grouping of items, based on one or more characteristics, attributes, properties, qualities, effects, parameters, etc., which they have in common, for the purpose of classifying them according to an established system or scheme. For example, subjects having gains associated with poor prognosis, such as gains in MCRs and/or genes listed in for example Tables 1, 2, 5, 9, 10 and/or 11, or losses of MCRs and/or genes listed in Table 3, 4 and/or 7, define a class with poor prognosis. Also for example, subjects having a gain in a Table 5, 9 or 11 gene or loss in a Table 7 gene identified as showing benefit from receiving chemotherapy, define a class that benefit from receiving chemotherapy. Similarly, subjects for example with a gain in a Table 6 gene or a loss of a Table 8 gene define a class with good prognosis.

The term “loss” or “gain” refers with respect to a genomic profile refers to a change in copy number, for example the loss can be on the plus strand or the minus strand and can involve loss of one or both alleles. Similarly, a “gain” can for example be a gain on the plus strand or the minus strand and can involve gain on one or both alleles. The gain can additionally be the gain of 1 or more copies.

The term “high amplitude gain” or “high level amplification” as used herein refers to a copy number variation of a MCR or gene amplification where the average log 2 value, as assigned by DNAcopy analysis, in the gained samples, was greater than 0.15. For example, high amplification gains were identified as described in the Examples and include for example MCRs listed in Table 10 and genes listed in Table 11.

The term “prognosing” as used herein means predicting clinical outcome such as survival and/or response to chemotherapy for example by identifying the class a subject belongs to according to the presence of a gain or loss of a genomic region such as 12q, 11q, 8q, 1p, or 14q or a region (MCR) or gene identified in any one of Tables 1 to 11. Where one or more gains or losses are detected, clinical outcome can be based on a subject's similarity to a control and/or a reference profile and/or biomarker expression level associated with a prognosis. Methods of prognosis described herein can optionally be included in multivariate models incorporating known prognostic clinical factors, such as age, sex stage and grade.

The term “good survival” as used herein refers to an increased disease free survival for example as compared to subjects in a suitable comparator “poor survival” group e.g. not having a gain or loss associated with good prognosis or improved response to chemotherapy. The term “poor survival” as used herein refers to an increased risk of death and/or disease occurrence as compared to subjects in a suitable comparator “good survival” group e.g. having a gain or loss associated with good prognosis or improved response to chemotherapy. For example, subjects comprising a gain or loss of a MCR or gene or altered biomarker expression described herein as associated with poor prognosis, such as genes and MCRs listed in Tables 1, 2, 5, 7, and/or 9-11, have a poor survival compared to subjects not comprising such a loss, gain or altered expression as indicated therein. As another example, subjects not receiving chemotherapy who comprise a gain or loss associated with improvement when treated with chemotherapy, for example such as MCRS listed in Table 1, 2, and/or 3 and/or genes listed in Tables 5, 7, 9 and/or 11 associated with improvement with chemotherapy, have poor survival when not treated with chemotherapy compared to subjects with the same gain, loss or altered expression who receive chemotherapy. As a further example, subjects who comprise a gain or loss not associated with improvement when treated with chemotherapy have a poor prognosis compared to individuals without the gain, or loss. Similarly, for example, a good survival group comprises subjects comprising a gain or loss or biomarker expression described herein associated with good prognosis, for example a gain or loss listed in Table 6 and/or 8 respectively. As another example, subjects receiving chemotherapy that comprise gains or losses associated with improved survival with chemotherapy, such as the particular MCRs listed in Table 1, 2 and/or 3 and/or the genes in Tables 5, 7, 9 and/or 11 identified as associated with significant improvement with chemotherapy have good survival when treated with chemotherapy compared to subjects with the similar gain, loss or expression who do not receive chemotherapy. Subjects in a good survival group or good survival group when treated with chemotherapy are at less risk of death 5 years after surgery. Subjects in a poor survival group or poor survival when not treated with chemotherapy group are at greater risk of death within 5 years from surgery. For example a poor survival group comprises subjects having a 5 year survival rate of less than 80%. As used herein, good survival indicates good prognosis and poor survival indicates poor prognosis.

The term “genes associated with good survival” or “genes associated with good prognosis” as used herein refers to genes listed in Table 6, for example RAB11FIP1 and genes listed in Table 8, for example, C6orf15, CDYL, HLA-DOA, KIFC1, MSH5/C6orf26, NCR3, RXRB, and/or TCL6.

The term “MCRs associated with good survival” or “MCRs associated with good prognosis” as used herein refer to MCRs associated with good prognosis for example the MCRs comprising the genes listed in Tables 6 and/or 8.

The term “genes associated with good survival when treated with chemotherapy” or “genes associated with good prognosis when treated with chemotherapy” as used herein refers to for example genes identified in Table 5 as showing significant improvement and/or trending to improvement, for example MFSD7, D4S234E, ACOX3, SRD5A1, AQP2, ACCN2, SLC11A2, SCN8A, KRT81, KRT1, ESPL1, NPFF, ATP5G2, HOXC11, NEUROD4, ZBTB39, KIAA0286, INHBE, MARS, B4GALNT1, TSFM, and/or DNMT3B; and/or genes listed in Table 7, for example RHOC, ATP2C2, ZDHHC7, COC4I1, and/or FOXF1; and/or gene listed in Table 9, for example BAALC, ANGPT1, MYC, WISP1, KRT81, KRT1, NEUROD4, and/or PA2G4; and/or genes listed in Table 11, for example, GUCA2A, PPIH, LEPRE1, CR623026, C1orf50, DQ515898, DQ515897, MYC FGF3, KRT81, KRT1, FAM112B, B4GALNT1, CENTG1, and/or BCL11B.

The term “genes associated with poor survival” or “genes associated with poor prognosis” alternatively “genes associated with poor survival/prognosis in the absence of chemotherapy” as used herein refers to for example genes so identified and listed in Table 5, for example MFSD7, D4S234E, ACOX3, SRD5A1, ADCY2, (clone Z146), ANKH, CDH18, OXCT1, UTRN, cDNA DKFZp434E2423, C9orf68, AQP2, ACCN2, SLC11A2, SCN8A, KRT81, KRT1, ESPL1, NPFF, ATP5G2, HOXC11, NEUROD4, ITGA7, CDK2/BCDO2, ERBB3, DLST/PA2G4, PRIM1, ZBTB39, KIAA0286, INHBE, MARS, B4GALNT1, TSFM, TRHDE, OR1E1/OR1E2, RCVRN, and/or DNMT3B; and genes listed in Table 7, for example AHCYL1, RHOC, ATP1A1, IGSF3, ELF1, RGC32, ESD, TAF1C, ATP2C2, ZDHHC7, COC4I1, FOXF1, and/or MAP1LC3B; and/or genes Table 9, including ANGPT1, HOXC11, ITGA7, PRIM1, B4GALNT1, OS9, CDK4, and TSFM; and/or genes in Table 11, including GUCA2A, LEPRE1, C1orf50, FGF3, FAM112B, B4GALNT1, OS9, CENTG1, CDK4, TSFM, AK024870, NUP107, MDM2, CPSF6, BCL11B, ASXH1 and/or C20orf112.

The term “genes not associated with improvement when treated with chemotherapy” as used herein refers to genes for example listed in Table 5 identified as not showing significant improvement when treated with chemotherapy, for example ADCY2, (clone Z146), ANKH, CDH18, OXCT1, UTRN, cDNA DKFZp434E2423, C9orf68, ITGA7, CDK2/BCDO2, ERBB3, DLST/PA2G4, PRIM1, TRHDE, OR1E1/OR1E2, and/or RCVRN; and/or genes listed in Table 7 identified as not showing significant improvement when treated with chemotherapy, for example AHCYL1, ATP1A1, IGSF3, ELF1, RGC32, ESD, TAF1C, and/or MAP1LC3B, as well as genes listed in Table 9 and/or 11 so identified. Detection of these genes for example is useful for selecting a treatment regimen. For example since subjects comprising losses or gains at these loci do not demonstrate improved prognosis with cisplatin, and/or venolrebine, chemotherapeutics that are not related to cisplatin and/or venolrebine e.g. a different class of drug, may be indicated.

The term “minimal common region” or “MCR” refers to the a region determined to be commonly gained or lost in subjects belonging to a particular class such as good prognosis when treated chemotherapy. A subject may have a gain or loss that comprises the MCR and/or comprises a portion of the MCR. For example the minimal common regions associated with poor prognosis in the absence of chemotherapy, and/or improved prognosis upon treatment with chemotherapy, are listed in Tables 1-11. The MCR start and stop positions refer to positions in NCBI human genome build 36.3 which corresponds to hg18.

As used herein, “treatment” or “treating” is an indicated approach for obtaining beneficial or desired results, including clinical results, for example an indicated approach for lung cancer. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, prolonging survival as compared to expected survival if not receiving treatment and remission (whether partial or total), whether detectable or undetectable. For example surgery is indicated for Stage I lung cancers, and surgery plus adjuvant chemotherapy is indicated for subjects with more advanced stages. The methods described herein are useful for example, for identifying subjects with lung cancer that benefit from receiving chemotherapy.

The phrase “selecting a treatment” as used herein refers to selecting a chemotherapeutic regimen, for example a regimen comprising a platinum based chemotherapeutic such as cisplatin, a regimen comprising a vinca alkyloid such as vinolrebine or a treatment regimen comprising a combination thereof, that is useful for obtaining beneficial results such as prolonging survival. Alternatively for example, where MCRs or genes that are not associated with improvement with chemotherapy or good prognosis, the treatment selected is a regimen that does not comprise a platinum based chemotherapeutic such as cisplatin, a regimen comprising a vinca alkyloid such as vinolrebine or a treatment regimen comprising a combination thereof.

The term “subject” such as a “subject” to be diagnosed, prognosed, staged, screened, assessed for risk, subject for selection of a treatment, and/or treated by the subject methods and articles of manufacture can mean either a human or non-human animal, preferably a human being.

The term “sample”, “test sample” or “biological sample” as used herein refers to any fluid, cell or tissue sample from a subject which can be assayed for genomic alterations or biomarker expression products e.g. for determining a genomic profile or an expression profile, depending on the method and comprises without limitation lung tumor tissue and/or cells, derived from, for example, lung biopsy, for example obtained by bronchoscopy, needle aspiration, thoracentesis and/or thoracotomy, and/or derived from cells found in sputum. The term could also be used for example to refer to metastatic tumour tissue obtained from the brain or liver or other site.

The phrase “determining the expression level of biomarkers” as used herein refers to determining or quantifying RNA and/or polypeptides expressed by the biomarkers. The term “RNA” includes mRNA transcripts, and/or specific spliced variants of mRNA. The term “RNA product of the biomarker” as used herein refers to RNA transcripts transcribed from the biomarkers and/or specific spliced variants. In the case of “polypeptide”, it refers to polypeptides translated from the RNA transcripts transcribed from the biomarkers. The term “polypeptide product of the biomarker” refers to polypeptide translated from RNA products of the biomarkers.

The term “nucleic acid” as used herein refers to a polynucleotide molecule and includes DNA and RNA and can be either double stranded or single stranded. The nucleic acid molecules contemplated by the present disclosure include isolated nucleotide molecules which hybridize specifically to genomic DNA, RNA product of a biomarker, polynucleotides which are complementary to a RNA product of a biomarker of the present disclosure, nucleotide molecules which act as probes, or nucleotide molecules which are specific primers for a MCR or gene gained or lost set out in Tables 1-11, including for example the probes and primers listed in Tables 12 and 13.

The term “isolated nucleic acid” as used herein refers to a nucleic acid substantially free of cellular material or culture medium when produced by recombinant DNA techniques, or chemical precursors, or other chemicals when chemically synthesized. An “isolated nucleic acid” is also substantially free of nucleotides which naturally flank the nucleic acid (i.e. nucleotides located at the 5′ and 3′ ends of the nucleic acid) from which the nucleic acid is derived.

The term “hybridize” refers to the sequence specific non-covalent binding interaction with a complementary nucleic acid. In a preferred embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. For example, 6.0× sodium chloride/sodium citrate (SSC) at about 45° C., followed by a wash of 2.0×SSC at 50° C. may be employed when hybridization is detecting expression levels, for example by northern or slot blot analysis. For array CGH, hybridization often occurs with labeled DNA for patient and reference DNA added to a solution including formamide and SSC (2.0×). The DNA/hybridization buffer mixture is allowed to competitively hybridize at 45° C. to the array (and its targets) for ˜36-40 hours, after which washes take place. Signal intensities at each arrayed element are then evaluated. A detailed description of array CGH hybridization protocols is provided in Buys et al., “Key Features of Bacterial Artificial Chromosome Microarray Production and Use” in DNA Microarrays (Methods Express Series) (Schena M, ed.), Scion Publishing, Ltd. Bloxham, Oxfordshire, UK, pp. 115-145 (ISBN: 9781904842156) (please see section 2.5 in particular).

The term “primer” as used herein refers to a nucleic acid sequence, whether occurring naturally as in a purified restriction digest or produced synthetically, which is capable of acting as a point of synthesis of when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less. The factors involved in determining the appropriate length of primer are readily known to one of ordinary skill in the art.

The term “primer pair” as used herein refers a set of primers which can produce a double stranded nucleic acid product complementary to a portion of the RNA products of the biomarker or sequences complementary thereof.

The term “probe” and/or “hybridization probe” as used herein refers to a nucleic acid sequence that will hybridize to a nucleic acid target sequence, for example. For example, the probe hybridizes to a RNA product of the biomarker or a nucleic acid sequence complementary thereof for detecting gene expression or hybridizes a genomic region comprising a gain or loss of a genomic region described herein associated with prognosis. The length of probe depends on the hybridization conditions and the sequences of the probe and nucleic acid target sequence. For example, the probe comprises at least 8, 10, 15, 20, 25, 50, 75, 100, 150, 200, 250, 400, 500 or more nucleotides in length, for example complementary to at least 8, 10, 15, 20, 25, 50, 75, 100, 150, 200, 250, 400, or 500 contiguous nucleotides of a gene listed in Table 5, 6, 7, 8, 9 and/or 11, or a genomic region alteration such as a MCR and/or region flanking a MCR described herein, for example in Tables 1 to 11, for example Table 1, 2, 3, 4 and/or 10. The probe can further be 90%, 95, 96, 97, 98, 99, 99.5, 99.9% identical to the at least 8, 10, 15, 20, 25, 50, 75, 100, 150, 200, 250, 400, or 500 contiguous nucleotides of a gene listed in Table 5, 6, 7, 8, 9 and/or 11, or a genomic region alteration such as a MCR and/or region flanking a MCR described herein, for example in Table 1, 2, 3, 4 and/or 10. The probe can also for example comprise a MCR or a gene associated with prognosis. For example the probe can be a bacterial artificial chromosome (BAC) clones and can comprise the target sequence as well as additional sequence. In this case, the probe can be at least 50 000, 100 000, 150 000 and/or 200 000 nucleotides, for example 150 000-200 000 base pairs The probe can for example comprised in an array, for example, on a solid support, for example array for CGH. For example, BAC clone probes on the array are usually in the 150,000-200,000 bp range. Labelled DNA and reference DNA generated from subject and reference DNA samples are typically a few hundred by in size (small fragments may be excluded after labeling or during washing steps). These subject DNA and reference DNA are generated for example, using a random priming reaction, such that their lengths will vary. See for example Buys et al. reference (above) and citations within (e.g. original citation at Feinberg & Vogelstein Anal. Biochem, 132, 6-13.)

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include Fab, Fab′, F(ab′)2, scFv, dsFv, ds-scFv, dimers, minibodies, diabodies, and multimers thereof and bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

The term “biomarker” as used herein refers to a gene that is altered in its gene copy number in a poor prognosis class and/or a good prognosis class e.g. with or without chemotherapy, compared to a control and/or is differentially expressed in subjects in poor and good prognosis classes. For example the term “biomarkers” includes one or more of the genes listed in Table 5, 6, 7, 8, 9 and/or 11.

The definitions and embodiments described in particular sections are intended to be applicable to other embodiments herein described for which they are suitable as would be understood by a person skilled in the art.

### II. Methods

Lung cancer remains the leading cause of cancer death in Canada with an overall 5-yr survival rate of 16%. Up to 40% of lung cancer patients are potentially curable by surgery, yet their risk of dying from the disease remains high at 50%. Post-surgery chemotherapy is a toxic therapy but may improve cure rate. New methods of classifying lung cancers are needed for making more informed decisions on chemotherapy, based on specific molecular markers present in each cancer. Using a CGH microarray, small regions of chromosomes have been identified that when gained or lost in lung cancers, impart a worse prognosis with surgery alone, and a subset of these also show a significant benefit with current standard chemotherapy. After testing individual genes within these regions by quantitative polymerase chain reaction, DNA copy number gains located on 1p, 8q, 11q, 12q, and 14q were confirmed to impart a worse prognosis in the absence of chemotherapy, and/or an improved response to chemotherapy.

Accordingly in an aspect, the disclosure provides a method for determining a lung cancer prognosis in a subject, the method comprising: determining a genomic profile comprising detecting one or more genomic alterations in chromosomes 2, 11, 4, 5, 7, 9, 12, 17, 19, 20, 8, 1, 13, 16, 6 and/or 14 listed in Tables 1-11 in a biological sample from the subject; wherein the prognosis is determined to be poor in the absence of chemotherapy when the genomic profile comprises a gain of one or more minimal common regions (MCRs) or genes within chromosomes 1, 2, 11, 12, 4, 5, 6, 7, 9, 12, 14, 16, 17, 19 and 20 listed as associated with poor prognosis in Tables 1, 2, 5, 9, 10, and 11, and/or a loss of one or more MCRs or genes within chromosomes 1, 5, 8, 13 and/or 16 listed as associated with poor prognosis in Tables 3 and/or 7 and the prognosis is determined to be good in the absence of chemotherapy when the genomic profile comprises a genomic gain of an MCR or gene within chromosome 8 listed as associated with good prognosis in Table 6 and/or a loss of one or more MCRs or genes within chromosome 2, 6, 9 or 14 listed as associated with good prognosis in Table 8 relative to the control.

In an embodiment, the method comprises: (a) determining a genomic profile comprising detecting one or more genomic alterations in chromosomes 2, 11, 4, 5, 7, 9, 12, 17, 19, 20, 8, 1, 13, 16, 6 and/or 14 listed in Tables 1-11 in a biological sample from the subject; (b) determining the lung cancer prognosis for the subject by comparing the genomic profile with one or more controls, wherein the prognosis is determined to be poor when the genomic profile comprises a gain of one or more minimal common regions (MCRs) or genes within chromosomes 1, 2, 11, 12, 4, 5, 6, 7, 9, 12, 14, 16, 17, 19 and 20 listed as associated with poor prognosis in Tables 1, 2, 5, 9, 10, and 11, and/or a loss of one or more MCRs or genes within chromosomes 1, 5, 8, 13 and/or 16 listed as associated with poor prognosis in Tables 3 and/or 7 and the prognosis is determined to be good when the genomic profile comprises a genomic gain of an MCR or gene within chromosome 8 listed as associated with good prognosis in Table 6 and/or a loss of one or more MCRs or genes within chromosome 2, 6, 9 or 14 listed as associated with good prognosis in Tables 6 and/or 8 relative to the control.

In an embodiment, the method comprises obtaining a biological sample for determining the genomic profile.

In another embodiment, the disclosure provides a method for determining a lung cancer prognosis in a subject, the method comprising: detecting the presence of a genomic alteration at a locus identified in Tables 1-11 in a biological sample from the subject, wherein the prognosis is determined to be poor in the absence of chemotherapy when a gain of a MCR or gene listed in Tables 1, 2, 5, 9, 10 and/or 11 and/or a loss of a MCR or gene listed in Table 3 and/or 7 is detected; and the prognosis is determined to be good when a gain of a MCR or gene listed in Table 6 and/or loss of a MCR or gene in Table 4 and/or 8 is detected relative to a control.

In an embodiment, the genomic alteration detected comprises a gain or loss of DNA copy number at an MCR listed in Tables 1-11, for example Table 1, 2, 3, 4 and/or 10. In another embodiment, the presence or absence of a gain of DNA copy number is detected at an MCR at 1p at or within basepair positions 41265460 to 43221579. In another embodiment, the presence or absence of a gain of DNA copy number is detected at an MCR at 8q at or within basepair positions 128289292 to 128936748. In another embodiment, the presence or absence of a gain of DNA copy number is detected at an MCR at 11q at or within basepair positions 68572940 to 70388868. In another embodiment, the presence or absence of a gain of DNA copy number is detected at an MCR at 14q at or within basepair positions 96994959 to about 99058653. In another embodiment, the presence or absence of a gain of DNA copy number is detected at an MCR at 12q at or within basepair positions 50731457 to 51457372. In another embodiment, the presence or absence of a gain of DNA copy number is detected at an MCR at 12q at or within basepair positions 52696908 to 53538441. In another embodiment, the presence or absence of a gain of DNA copy number is detected at an MCR at 12q at or within basepair positions 55933813 to 57461765. In another embodiment, the presence or absence of a gain of DNA copy number is detected at an MCR at 12q at or within basepair positions 64438067 to 68503251. In another embodiment, the presence or absence of a gain of DNA copy number is detected at an MCR at 14q at or within basepair positions 96994959 to 99058653.

In another embodiment, the genomic alteration detected comprises all or part of a MCR listed in Table 1, 2, 3, 4 and/or 10. In an embodiment, the genomic alteration detected comprises all or part of a MCR listed in Table 10.

In an embodiment, the method comprises determining a genomic profile comprising detecting one or more genomic alterations listed Table 1, 2, 5, 9, 10 and/or 11, in a biological sample from the subject; (b) determining the lung cancer prognosis for the subject by comparing the genomic profile with one or more controls, wherein the prognosis is determined to be poor in the absence of chemotherapy when the genomic profile comprises a gain of one or more minimal common regions (MCRs) or genes listed in Table 1, 2, 5, 9, 10 and/or 11.

In another embodiment, the method comprises determining a genomic profile comprising detecting one or more genomic alterations in chromosomes 1, 5, 8, 13 and 16 listed in Table 3 and/or 7 wherein the prognosis is determined to be poor in the absence of chemotherapy when the genomic profile comprises a loss of one or more MCRs or genes within chromosomes 1, 5, 8, 13 and 16 listed in Table 3 and/or 7.

In a further embodiment, the method comprises determining a genomic profile comprising detecting a genomic alteration or gene gain in chromosome 8 listed as associated with good prognosis in Table 6, wherein the prognosis is determined to be good when the genomic profile comprises a gain of the MCR or the gene within chromosome 8 listed as associated with good prognosis.

In another embodiment, the method comprises determining a genomic profile comprising detecting one or more genomic alterations in chromosomes 6 and/or 14, wherein the prognosis is determined to be good when the genomic profile comprises a loss of one or more MCRs within chromosomes 6 and/or 14 listed in Table 8.

In another aspect, all or part of genes located within the MCRs gained or lost, for example the MCRs listed in any one of Tables 1 to 11, for example, Tables 1, 2 and/or 10 are detected. Detection of an increased or decreased DNA copy number of a gene (e.g. a gain, amplification, or loss of said gene) comprised therein can be indicative of the presence or absence of a gain, amplification, or loss at the corresponding MCR. For example, DQ515898, DQ515897, and MYC genes are found within the MCR at basepair positions 128289292 to 128936748 on chromosome arm 8q, CCND1 and FGF3 genes are found within the MCR at basepair positions 68572940 to 70388868 on chromosome arm 11q, and B4GALNT1, OS9, CENTG1, CDK4, and TSFM are genes found within the MCR at basepair positions 55933813 to 57461765 on chromosome arm 12q.

In an embodiment, the gene detected is selected from the group, DQ515898, DQ515897, and MYC. In a further embodiment, the gene detected is selected from the group consisting of AK024870, NUP107, MDM2, CPSF6, and BCL11B. In a further embodiment, the gene detected is selected from the group consisting of GUCA2A, PPIH, LEPRE1, CR623026, and C1orf50. In a further embodiment, the gene detected is selected from the group consisting of CCND1 and FGF3. In a further embodiment, the gene detected is selected from the group consisting of B4GALNT1, OS9, CENTG1, CDK4, and TSFM.

In another embodiment, the method comprises detection of a gain of all or part of one or more of the genes listed in Table 9 and/or 11 for genes identified as associated significantly with poor prognosis (and/or trending to poor prognosis) including ANGPT1, HOXC11, ITGA7, PRIM1, B4GALNT1, OS9, CDK4, and TSFM (e.g. Table 9 genes) and/or GUCA2A, LEPRE1, C1orf50, FGF3, FAM112B, B4GALNT1, OS9, CENTG1, CDK4, TSFM, AK024870, NUP107, MDM2, CPSF6, BCL11B, ASXH1 AND C20orf112 (e.g. Table 11 genes).

The MCRs described herein as associated with prognosis comprise gains or losses of genes listed in Tables 5, 6, 7, 8, 9 and/or 11, and of the genomic regions listed in Tables 1 to 11 and particularly Tables 1, 2, 3, 4 and/or 10. The gain or loss can be all or part of any one of these genes. In an embodiment, the detected gain or loss comprises amplification and/or deletion of the entire gene.

Accordingly, in a further embodiment, the prognosis is determined to be poor, in the absence of chemotherapy, when the genomic profile comprises a gain of a MCR comprising all or part of a gene listed in Table 5, 9 and/or 11 associated with poor prognosis and/or comprises a loss of a MCR comprising all or part of a gene listed in Table 7 associated with poor and/or comprises a gain of an MCR in table 1, 2 and/or 10 associated with poor prognosis, and the prognosis is determined to be good, in the absence of chemotherapy, when the genomic profile comprises a gain of a MCR comprising all or part of gene listed in Table 6 and/or a loss of a MCR comprising all or part of a gene listed in Table 8 relative to the control.

The genomic profile can be determined by various methods for example by determining a hybridization pattern using a probe that hybridizes to a region described herein as associated with a prognosis or outcome. In an embodiment, a set of probes are used. In another embodiment the probe is a chromosomal probe.

In an embodiment, detection of one of the gains losses described herein is sufficient for association with prognosis and/or response to chemotherapy.

In an embodiment, the method comprises hybridizing a chromosomal probe or a set of chromosomal probes to the biological sample, and detecting the presence or absence of hybridized probe.

In an embodiment, the probe is complementary to at least 8, 10, 15, 20, 25, 50, 75, 100, 150, 200, 250, 400, 500 contiguous nucleotides of a gene listed in Table 5, 6, 7, 8, 9 and/or 11, or a genomic region alteration such as a MCR and/or region flanking a MCR described herein, for example in Table 1, 2, 3, 4 and/or 11. In another embodiment, the probe is at least or greater than 90, 95, 96, 97, 98, 99, 99.5 or 99.9% identical to a gene listed in Tables 5, 6, 7, 8, 9 and/or 11, or a region in listed in any one of Tables 1-11, for example Tables 1, 2, 3, 4 and/or 10.] Alternatively, for example the probe can be a bacterial artificial chromosome (BAC) clone and can comprise the target sequence. In this case, the probe can be at least 50,000 bp, at least 100,000 bp, at least 150,000 bp and/or at least 200 000 bp, for example 150 000-200 000 bp The probe can for example comprised in an array, for example, on a solid support. Accordingly, in another embodiment, the set of chromosomal probes is comprised in an array.

In a further embodiment, the probes are labeled, for example the probes are fluorescently labeled. In another embodiment, the subject DNA and the reference DNA is labeled.

Accordingly in another embodiment, the method comprises: (a) determining a hybridization pattern of a chromosomal probe in a biological sample from the subject, wherein the probe hybridizes to a chromosome selected from the group 11, 4, 5, 6, 7, 9, 12, 17, 20, 8, 1, 13, 16, and/or 14 and (b) determining the lung cancer prognosis for the subject based on the hybridization pattern, wherein the prognosis is determined to be poor when the hybridization pattern indicates a gain of one or more MCRs or genes within chromosome for example 1, 2, 8, 11, 12, 14 and/or 20 listed in Table 1, 2 and/or 10, or for example within chromosome 1, 2, 4, 5, 6, 7, 8, 9, 11, 12, 14, 17, and 20 listed in Table 5, 9 and/or 11 and/or a loss of one or more MCRs or genes within chromosomes 1, 13 and 16 listed in Table 7 and the prognosis is determined to be good when the hybridization profile indicates a gain of a MCR or gene within chromosome 8 in Table 6 and/or a loss of one or more MCRs or genes within chromosome 6 or 14 in Table 8, relative to the control.

Accordingly in another embodiment, the method comprises: (a) determining a hybridization pattern of a chromosomal probe or set of chromosomal probes in a biological sample from the subject, wherein the set comprises one or more probes directed to one or more MCRs and/or genes in chromosomes 2, 11, 4, 5, 6, 7, 9, 12, 14, 16, 17, 19, 20, 8, 1, 13, 16, 6 and/or 14 listed in Tables 1-11; and (b) determining the lung cancer prognosis for the subject based on the hybridization pattern, wherein the prognosis is determined to be poor when the hybridization pattern indicates a gain or loss of one or more MCRs or genes associated with poor prognosis and the prognosis is determined to be good when the hybridization profile indicates a gain or loss of one or more MCRs or genes associated with good prognosis relative to the control.

In an embodiment, the prognosis is determined to be poor when the hybridization pattern indicates a gain of one or more MCRs or genes listed in Table 1, 2, 5, 9 and/or 11 and/or a loss of one or more MCRs or genes listed in Table 3 and/or 7. In an embodiment, the gain comprises all or part of a gene listed in Table 5. In another embodiment, the gain comprises all or part of a gene listed in Table 9. In yet another embodiment, the gain comprises all or part of a gene listed in Table 11. In another embodiment, the loss comprises all or part of a gene listed in Table 7.

In yet a further embodiment, the prognosis is determined to be good when the hybridization pattern indicates a gain of a MCR or gene within chromosome 8 and/or a loss of one or more MCRs or genes within chromosome 6 or 14 relative to the control. In an embodiment, the gain comprises all or part of RAB11FIP1. In another embodiment, the loss comprises all or part of a gene listed in Table 8.

It has also been determined that subjects with a gain or loss of a subset of MCRs or genes are associated with significant improvement in survival and/or have improved tumor responsiveness with chemotherapy compared to subjects with the gain or loss not treated with chemotherapy.

In another aspect, the disclosure includes a method for determining a likelihood of improved survival or response with chemotherapy treatment comprising detecting a gain of all or part of a MCR or gene listed in Tables 1, 2, 5, 9, 10 and/or 11 associated with improved response to chemotherapy, wherein a gain indicates the subject has a good prognosis when treated with chemotherapy relative to a subject not treated with chemotherapy.

In another aspect, the disclosure includes a method for determining tumour responsiveness to a chemotherapy treatment comprising detecting a gain of all or part of one or more of the genes listed in Tables 1, 2, 5, 9 or 11 associated with improved response to chemotherapy, wherein a gain indicates the tumour is likely responsive to treatment with chemotherapy relative to a tumour not comprising the gain.

Accordingly in an embodiment, a gain detected of all or part of one or more of the following genes: MFSD7, D4S234E, ACOX3, SRD5A1, AQP2, ACCN2, SLC11A2, SCN8A, KRT81, KRT1, ESPL1, NPFF, ATP5G2, HOXC11, NEUROD4, ZBTB39, KIAA0286, INHBE, MARS, B4GALNT1, TSFM, DNMT3B and/or the loss of all or part of one of the following genes: RHOC, ATP2C2, ZDHHC7, COC4I1, FOXF1, indicates the subject has a good prognosis when treated with chemotherapy relative to a subject not treated with chemotherapy.

In another embodiment, the gain associated with improved survival with chemotherapy or improved tumor responsiveness is a gain of all or part of one or more of the following genes: BAALC, ANGPT1, MYC, WISP1, KRT81, KRT1, NEUROD4, and/or PA2G4 (e.g. Table 9 genes associated with improved response to chemotherapy). In a further embodiment, the gain associated with improved survival with chemotherapy or improved tumor responsiveness is a gain of all or part of one or more of the following genes: GUCA2A, PPIH, LEPRE1, CR623026, C1orf50, DQ515898, DQ515897, MYC FGF3, KRT81, KRT1, FAM112B, B4GALNT1, CENTG1, BCL11B (e.g. Table 11 genes associated with improved response to chemotherapy).

Another aspect provides a method of determining a lung cancer prognosis in a subject, the method comprising detecting the presence of a MCR and/or gene associated with improvement with chemotherapy, for example a MCR of Table 1, 2, and/or 3, or a gene from Table 5 or 7, wherein the gain or loss of a MCR and/or gene associated with improvement with chemotherapy (as indicated in the relevant table) is indicative the subject will have good prognosis relevant to a control, for example a subject with the gain or loss not receiving chemotherapy.

In another aspect, the disclosure includes a method for determining a likelihood of improved survival with chemotherapy treatment comprising detecting a loss of all or part of a MCR or gene listed in Tables 3, 4, 7 and/or 8 associated with improved response to chemotherapy, wherein the loss indicates the subject has a good prognosis when treated with chemotherapy relative to a subject not treated with chemotherapy.

In another aspect, the disclosure includes a method for determining tumour responsiveness to a chemotherapy treatment comprising detecting a loss of all or part of a MCR or gene listed in Tables 3, 4, 7 and/or 8 associated with improved response to chemotherapy, wherein the loss indicates the tumour is likely responsive to treatment with chemotherapy relative to a tumour not comprising the loss.

In an embodiment, the chemotherapy comprises a platinum based chemotherapeutic. In another embodiment, the chemotherapy comprises a vinca alkaloid. In a further embodiment, the chemotherapy regimen includes both a platinum based chemotherapeutic and a vinca alkyloid.

Expression data of the genes herein identified associated with prognosis is also predicted to be useful for predicting prognosis. Generally, with increasing gene dosage, gene expression levels would be expected to increase. Similarly, with decreasing gene dosage, gene expression would be expected to decrease. This is for example often the case with heterozygous gene knock out in mice, and/or transgene copy number in transgenic mice. For example, increased expression of a gene whose gain is associated with poor outcome is expected to be indicative of poor outcome and decreased expression of a gene, loss of which is associated with poor outcome is expected to be indicative of poor outcome. Similarly, increased expression of a gene, gain of which is associated with good outcome is expected to be indicative of good outcome and decreased expression of a gene, loss of which is associated with good outcome, is expected to be indicative a good outcome. Gene expression can be determined alone and/or in conjunction with genomic alterations.

Accordingly, another aspect provides a method for determining a lung cancer prognosis in a subject, the method comprising: (a) determining an expression profile comprising detecting an expression level of one or more genes listed in Tables 5, 6, 7, 8, 9 and/or 11 associated with prognosis in a biological sample from the subject; wherein the prognosis is determined to be poor when the expression profile comprises a increased level of expression of one or more genes in Table 5, 9 and/or 11 associated with poor prognosis and/or a decreased expression in one or more genes listed in Table 7 and the prognosis is determined to be good when the expression profile comprises increased expression of RAB11FIP1 and/or decreased expression of one or more genes in Table 8, relative to a control.

In an embodiment, the method includes step (b), said step (b) comprising determining the lung cancer prognosis for the subject by comparing the expression profile with one or more controls.

The expression level is optionally determined in addition to the genomic copy number. Accordingly, in addition to determining the genomic profile and/or the detecting the gain or loss of a MCR comprising all or part of one or more genes listed in Tables 5, 6, 7, 8, 9 and/or 11, the method further comprises detecting the expression level of a gene listed in Table 5, 6, 7, 8, 9 and/or 11. In an embodiment, the expression level of the gene all or partly gained or lost, is increased or decreased respectively, relative to a control expression level.

In an embodiment, the expression level is detected using a probe that binds a gene listed in Tables 5, 6, 7, 8, 9 and/or 11. In an embodiment, the probe comprises at least 8, 10, 15, 20, 25, 50, 75, 100, 150, 200, 250, 400, 500 contiguous nucleotides complementary to a gene listed in Table 5, 6, 7, 8, 9 and/or 11, or a gene with at least 90, 95, 98, 99, 99.5 or 99.9% identity to a gene in Table 5, 6, 7, 8, 9 and/or 11. The probe can for example be comprised in an array, for example, on a solid support, for example array. In another embodiment, the expression level is detected by detecting the presence or absence of hybridized probe.

In a further embodiment, the probes are comprised in an array, for example on a solid support. In another embodiment, the probes are labeled or for example fluorescently labeled.

As described herein and mentioned above, prognostic associations have been found for MCRs of gain located on 12q and 14q (e.g. Table 1 or 10). Such MCR gains were found by array-CGH and qPCR studies to be significantly associated with poor survival in the absence of chemotherapy. Predictive associations have been found for MCRs of gain located on 1p, 8q, 11q, 12q, and 14q. Subjects with these MCRs were found to have improved survival when treated with chemotherapy.

Accordingly, another aspect provides a method for determining a lung cancer prognosis in a subject, the method comprising: (a) determining a hybridization pattern of a chromosomal probe in a biological sample from the subject, wherein the set comprises a probe to the 6 Mb region of chromosome 12q, 8q or 11q; and (b) determining the lung cancer prognosis for the subject based on the hybridization pattern, wherein the prognosis is determined to be poor without chemotherapy when the hybridization pattern indicates a gain of a MCR within the 6 Mb region of chromosome 12q relative to a control and/or the prognosis is determined to be good when treated with chemotherapy when the hybridization pattern indicates a gain of a MCR within 8q and/or 11q.

In an embodiment, gain of DNA copy number at an MCR located on 1p within basepair positions 41265460 to 43221579 is indicative of a good prognosis with chemotherapy.

In another embodiment, gain of DNA copy number at an MCR within basepair positions 128289292 to about 128936748 on the long arm of chromosome 8 is indicative of a good prognosis with chemotherapy.

In another embodiment, gain of DNA copy number at an MCR within basepair positions 68572940 to about 70388868 on the long arm of chromosome 11 is indicative of a good prognosis with chemotherapy.

In another embodiment, gain of DNA copy number at an MCR within basepair positions 50731457 to about 51457372 on the long arm of chromosome 12 is indicative of a good prognosis with chemotherapy.

In another embodiment, gain of DNA copy number at an MCR within basepair positions 52696908 to about 53538441 on the long arm of chromosome 12 is indicative of a good prognosis with chemotherapy.

In another embodiment, gain of DNA copy number at an MCR within basepair positions 55933813 to about 57461765 on the long arm of chromosome 12 is indicative of a good prognosis with chemotherapy.

In another embodiment, gain of DNA copy number at an MCR within basepair positions 96994959 to about 99058653 on the long arm of chromosome 14 is indicative of a good prognosis with chemotherapy.

Several genes comprised within the 1p, 8q, 11q, 12q, and 14q MCRs were also detected in a separate gene analysis. Accordingly, in an embodiment, the method comprises detection of DNA copy number of a gene in Tables 5-11 that falls within a MCR listed in Table 1, 2, 3, 4 and/or 10.

As a number of genome gains and losses are associated with tumour responsiveness and/or better survival when subjects are treated with chemotherapy, the disclosure provides methods for selecting a treatment for subjects with lung cancer.

Accordingly, in another aspect, the disclosure provides a method of selecting a treatment regimen for a subject with lung cancer, the method comprising: (a) determining a genomic profile comprising detecting a genomic alteration in one or more genes selected from Table 5 and/or 7 in a biological sample from the subject; (b) selecting a treatment for the subject optionally by comparing the genomic profile with one or more controls, wherein the treatment selected comprises chemotherapy when the genomic profile comprises a gain of all or part of one or more of the following genes: MFSD7, D4S234E, ACOX3, SRD5A1, AQP2, ACCN2, SLC11A2, SCN8A, KRT81, KRT1, ESPL1, NPFF, ATP5G2, HOXC11, NEUROD4, ZBTB39, KIAA0286, INHBE, MARS, B4GALNT1, TSFM, DNMT3B; and/or a loss of all or part of one or more of the following genes: RHOC, ATP2C2, ZDHHC7, COC4I1, and/or FOXF1 relative to the control.

In another embodiment, the gain associated with improved survival with chemotherapy or improved tumor responsiveness is a gain of all or part of one or more of the following genes: BAALC, ANGPT1, MYC, WISP1, KRT81, KRT1, NEUROD4, and/or PA2G4 (e.g. Table 9 genes associated with improved response to chemotherapy). In a further embodiment, the gain associated with improved survival with chemotherapy or improved tumor responsiveness is a gain of all or part of one or more of the following genes: GUCA2A, PPIH, LEPRE1, CR623026, C1orf50, DQ515898, DQ515897, MYC FGF3, KRT81, KRT1, FAM112B, B4GALNT1, CENTG1, BCL11B (e.g. Table 11 genes associated with improved response to chemotherapy).

In an embodiment, the gain comprises a gain in all or part of one or more of FGF3, FAM112B, TSFM, NUP107 and/or MDM2.

In an embodiment, the subject has been treated by surgical resection.

Two genes were identified as trending to worse survival with administration of chemotherapy.

Accordingly, in an embodiment the method for selecting a treatment comprises: (a) determining a genomic profile comprising detecting a genomic alteration in one or more genes selected from AK024870 and CPSF6; wherein the treatment selected comprises non-chemotherapy and/or a non-platinum analog-, vinca alkaloid or combination thereof chemotherapy treatment when the genomic profile comprises a gain of all or part of one or more of AK024870 and CPSF6.

The disclosure also provides a method of prognosis of likelihood of improved survival in a lung cancer subject who was and/or is receiving a chemotherapeutic treatment, comprising determining the presence or absence of a gain or loss of a MCR associated with improvement with chemotherapy, predicting the likelihood of improved survival according to the presence or absence of the MCR or gene gain or loss compared to a control, wherein detecting a MCR or gene associated with improvement with chemotherapy predicts likelihood of improved survival compared to a control having the same gain or loss who has not received or is not receiving chemotherapy.

In an embodiment, the presence of a gain or loss associated with improvement with chemotherapy is indicative of a favourable predisposition of the subject to respond to platinum analogs, vinca alkyloids and/or a combination thereof.

Another aspect provides a method of treating lung cancer comprising determining the presence or absence of a gain or loss of a MCR or gene associated with improvement with chemotherapy in a subject with lung cancer and administering chemotherapy to a subject with at least one gain or loss associated with improvement with chemotherapy.

In an embodiment the chemotherapy administered is a platinum analog, a vinca alkyloid or a combination thereof. In a further embodiment, the platinum analog is selected from the group consisting of cisplatin, paraplatin, carboplatin, oxaliplatin and satraplatin in either IV or oral form. In another embodiment the vinca alkyloid is selected from the group vinorelbine, vincristine, vinblastine, vindesine and vinflunine in either IV or oral form.

The methods described herein are useful for different lung cancers. In an embodiment, the lung cancer is non-small cell lung cancer (NSCLC), early stage NSCLC, squamous cell carcinoma, adenocarcinoma, or large cell carcinoma.

The biological sample can be any sample that comprises a polynucleotide or biomarker expression product to be assayed. In an embodiment, the biological sample is selected from the group consisting of lung tissue, lung cells, lung biopsy and sputum, including formalin fixed, paraffin embedded and fresh frozen specimens.

The methods described herein compare a subject profile, genomic or expression with a control. The control with respect to genomic alterations is for example the copy number of gene or region in a subject in a different class e.g. good prognosis when treated with chemotherapy versus poor prognosis when not treated with chemotherapy, or alternatively can be an internal control, e.g. the copy number at a region with no gain or loss, for example centromere copy number. For example, For the FISH method, the centromere copy number can be used. For the qPCR method, centromere cannot be used, and instead a “control” gene would be used, a gene on the same or different chromosome that is infrequently gained or lost. For array-CGH, a reference genomic DNA sample from a “normal” individual without cancer would be used. A person skilled in the art would be able to select an appropriate control. Accordingly, in an embodiment the control is the centromere copy number. Typically, the copy number of a gene or region is 2, one copy per allele. Accordingly, in another embodiment the control is such that a copy number greater than 2 is a gain, and a copy number less than 2 is a loss. Myc and CCDN1 have for example, previously been shown to be amplified in lung cancer, however it is not believed that they have been identified in association with improved response to chemotherapy.

In an embodiment, for example pertaining to prognosis without chemotherapy, the gene detected is not EGFR, MET, MYC, CCND1, KRAS, and/or TITF1.

### III. Compositions and Kits

The disclosure also provides compositions and kits which are useful for example in the methods described herein.

An aspect provides a composition comprising a detection agent for detecting the presence or absence of a MCR or gene gain or loss associated with prognosis. In an embodiment the detection reagent is a hybridization probe, for example a chromosomal probe or a gene expression probe. In an embodiment, the probe comprises at least 8, 10, 15, 20, 25, 50, 75, 100, 150, 200, 250, 400, or 500 contiguous nucleotides complementary to a gene listed in Table 5, 6, 7, 8, 9 and/or 11, or a genomic region alteration such as a MCR and/or region flanking a MCR described herein, for example in Tables 1 to 11, or for example in Table 1, 2, 3, 4 and/or 11. The probe can further be 90, 95, 96, 97, 98, 99, 99.5, 99.9% identical to the at least 8, 10, 15, 20, 25, 50, 75, 100, 150, 200, 250, 400, or 500 contiguous nucleotides of a gene listed in Table 5, 6, 7, 8, 9 and/or 11, and/or a MCR and/or region flanking a MCR described herein, for example in Table 1, 2, 3, 4 and/or 10. Depending on the probe type (e.g. oligonucleotide or BAC clone), the nucleotide length of the probe can vary, and in the case of a BAC clone can include sequence in addition to the gene or MCR associated. In an embodiment, the probe is a BAC clone. In an embodiment, the BAC clone is at least 50 000, 100 000, 150 000 or 200 000 nucleotides. In an embodiment the BAC clone is about 150 000-200 000 nucleotides. BAC clones can be used for example as probes in FISH and some array CGH platforms. In an embodiment, the probe is complementary to a MCR described herein. In a further embodiment the probe comprises a BAC clone that overlaps the MCR or gene gained or lost. In an embodiment the probe comprises the nucleotide sequence of a BAC clone of an Affymetrix U133A chip comprising a MCR or gene gain or loss described herein as associated with prognosis. A person skilled in the art on the basis on the teachings herein, such as the teachings in the Examples, would be able to identify the probes that correspond to the particular MCRs and genes.

In another embodiment, the composition comprises a primer or a primer pair for amplifying a biomarker expression polynucleotide, or a genomic region described herein. The primer is in an embodiment, 15-20, 21-30, 31-40, 41-50 or more than 50 nucleotides in length.

In an embodiment the composition further comprises a carrier.

In another aspect, the disclosure provides a kit for determining lung cancer prognosis in a subject comprising for example a detection agent or composition described herein. In an embodiment, the kit comprises a chromosomal probe wherein the probe hybridizes all or part of a MCR listed in Tables 1 to 11, for example in Table 1, 2, 3, 4 and/or 10 and/or all or part of a gene listed in Tables 5, 6, 7, 8, 9 and/or 11.

In another aspect, the disclosure provides a kit for determining lung cancer prognosis in a subject, the kit comprising one or more gene expression probes, wherein the set comprises a probe specific for a gene expression product of a gene listed in Tables 5, 6, 7, 8, 9 and/or 11.

In an embodiment, the probes are labeled, for example, the probes are fluorescently labeled. In other embodiment, the kit comprises labeling reagents for example for labeling subject sample, e.g. subject DNA.

In another embodiment, the probes are comprised in an array on a solid support.

In an embodiment, the kit comprises reagents for FISH analysis of a MCR or gene gain or loss described herein, and a control region such as a centromere or gene on the same or different chromosome. For example, the kit comprises a probe for a MCR or gene gain or loss described herein, and a reference probe to the centromere or a gene on the same or different chromosome, and labeling reagents for labeling the probe.

In another embodiment, the kit comprises reagents for CGH analysis of a MCR or gene gain or loss described herein, for example, the kit comprises an array with one or more probes for one or more MCRs or genes gained or lost described herein and labeling reagents for labeling the subject sample DNA.

In a further embodiment the kit comprises reagents for PCR such as quantitative or multiplex PCR. For example the kit comprises a primer set for amplifying all or part of a MCR or gene, or multiple MCRs or genes, described herein associated with prognosis, as well as one or more primer sets for identifying one or more control genes on the same or different chromosomes.

In yet a further embodiment, the kit comprises a primer set and probe for detecting an amplification product.

In a further embodiment, the kit comprises a positive and/or a negative control. The control in an embodiment comprises normal reference DNA for CGH or FISH based kits. A positive control comprises a tumour that is known to have a gain or loss at the particular target being assayed.

In yet a further embodiment, the kit further comprising instructions that indicate prognosis is determined to be poor in the absence of chemotherapy when a hybridization pattern of the chromosomal probe or set of chromosomal probes indicates a gain in a MCR in for example, chromosome 11 or 12 listed in Table 1, 2 and/or 10, or a gain in a MCR comprising all or part of a gene listed in Table 5, 9 and/or 11 and/or a loss of the a MCR comprising all or part of a gene listed in Table 7, relative to control; good when a hybridization pattern of one or more chromosomal probes indicates a gain in a MCR comprising all or part of a gene listed in Table 6 and/or a loss of the MCR comprising all or part of a gene listed in Table 8. In another embodiment, the kit comprises instructions that indicate prognosis is determined to good when treated with chemotherapy when a hybridization pattern of the chromosomal probe or set of chromosomal probes indicates a gain in a MCR comprising for example, all or part of MFSD7, D4S234E, ACOX3, SRD5A1, AQP2, ACCN2, SLC11A2, SCN8A, KRT81, KRT1, ESPL1, NPFF, ATP5G2, HOXC11, NEUROD4, ZBTB39, KIAA0286, INHBE, MARS, B4GALNT1, TSFM, and/or DNMT3B, and/or a loss in a MCR comprising all or part of RHOC, ATP2C2, ZDHHC7, COC4I1, FOXF1, relative to a control. In another embodiment, the kit comprises instructions that indicate prognosis is determined to be good when treated with chemotherapy, when a hybridization pattern of a chromosomal probe or set of chromosomal probes indicates a gain in a MCR comprising for example a gain listed in Table 9 and/or 11 to be associated with poor prognosis. In an embodiment, the instructions include direction for comparing to a control. In an embodiment, the instructions include direction and/or reagents for using a centromere copy number or other chromosome as a control.

The following non-limiting examples are illustrative of the present disclosure:

### EXAMPLES

### Example 1

### Results

Array-CGH and RNA Microarray:

The chromosomal pattern of observed gains and losses by array-CGH are in concordance with previous array-CGH and CGH studies in NSCLC, including frequent gains at chromosome 1q, 3q, 5p, and 8q, and frequent losses at 3p, 5q, 6q, 8p, 9p, 13q, and 17p. MCRs of DNA copy number alteration encompass multiple genes known to be important in NSCLC, including MYC, hTERT, and cyclin D1, as well as many potentially important novel genes.

Upon integration of wide MCRs of gain with RNA expression microarray data, there are 38 genes that, when gained in copy number, were found to impart a significantly worse survival in the absence of chemotherapy (p<0.05) (Table 5). These genes are found mostly on chromosomes 12q and 5p. Of these 38 genes 22 were found to show a significant improvement with chemotherapy by the interaction terms analysis on the array-CGH dataset. Only one gene (RAB11FIP1) was found to have a favourable effect on prognosis when gained (Table 6).

Within the wide MCRs of loss, 13 genes had a significant deleterious effect on survival in the absence of chemotherapy, predominantly found on chromosomes 1p, 13q, and 16q (Table 7). Of these, 6 genes were found to show a significant improvement with chemotherapy by the interaction terms analysis. Eight genes, mostly on chromosome 6p, showed an improved prognosis with loss of DNA material in one of the 3 analyses.

After removing known human copy number variations, 27 narrow MCRs of gain and 19 narrow MCRs of loss across the genome were identified for statistical analysis. After correcting for multiple testing, MCRs of gain within a 6 Mb region of 12q were found to be significantly associated with poor survival in the absence of chemotherapy (p<0.001, q<0.05). When this region was examined for benefit of chemotherapy, a significant improvement of survival was identified at one of these 12q MCRs (interaction p<0.01), while the other 12q MCRs showed a trend towards improved response to chemotherapy (Table 1). These associations remained significant (p<0.05) in a multivariate model incorporating known prognostic clinical factors (i.e., age, sex, stage, grade). Approximately 25% of samples showed gains at these MCRs on 12q, which were more common in squamous cell carcinomas (40%) than adenocarcinomas (20%), and tended to be seen in older patients.

Other potential predictive associations arising from this analysis that were not significant after multiple testing corrections included an improved survival with chemotherapy for patients with gains at MCRs on 8q (interaction p=0.02) and 11q (interaction p=0.08). The 11q gain showed significant predictive ability in the multivariate model (interaction p=0.02), whereas the MCR on 8q lost its predictive ability in the multivariate model in this analysis.

One hundred and twenty-three focal high-amplitude MCRs were identified from the 113 NSCLC samples interrogated by array-CGH. These amplicons were found on all 22 chromosomes examined, and included well-known amplified genes in NSCLC including EGFR, MET, MYC, CCND1, KRAS, and TITF1. Twenty-six of these high-amplitude MCRs were found to be well known copy number variations (CNVs) contained within the Database of Genomic Variants (DGV). Eleven of these MCRs were selected for further validation studies based on significant survival associations (Table 10).

Quantitative Polymerase Chain Reaction (qPCR):

There were 40 genes on chromosomes 5, 8, and 12 from the wide MCRs analysis, that were tested by qPCR on the same samples. Of these, 6 genes showed a significant (p<0.05) poor survival in the observation arm associated with DNA copy number gains as detected by qPCR (Table 9). Five of the genes showed a significant (p<0.05) improved outcome with chemotherapy by interaction terms analysis (Table 9). These survival associations were in agreement with the array-CGH analysis. However, the remainder of the genes tested did not show the same survival association by qPCR as by array-CGH, on DNA from the same samples.

Upon examination of the minority of genes that were validated by qPCR, it was noted that these genes tended to fall in regions that showed high-level amplifications. As a result of this finding, an array-CGH analysis designed to focus on high-level amplifications was performed, resulting in the list of high-amplitude MCRs listed in Table 10.

From the 11 prognostic/predictive high-amplitude MCRs, 38 genes have been tested by qPCR on the same samples. Of these, 16 have shown significant (p<0.05) survival associations (prognostic in the absence of chemotherapy, and/or predictive of improved response to chemotherapy) in agreement with the array-CGH analysis. An additional 9 of these genes show a trend to significant survival associations (p<0.2). Many of the genes with significant survival associations were found within the four 12q amplicons, showing a poor prognosis in the observation arm, and an improved response to chemotherapy.

### Discussion

High-resolution array-CGH analyses on a subset of the BR 10 patients have identified regions of recurrent copy number gain that may be predictive of benefit from adjuvant chemotherapy. This information would be very useful for selecting those lung cancer patients who should receive current adjuvant chemotherapy, those who do not require chemotherapy, and those patients who will require more experimental treatments in hopes of curing their disease. Further experiments are underway to validate these results in additional samples from the same study, as well as to identify critical genes in these areas. (Supported by grants from the Canadian Cancer Society, Ontario Institute of Cancer Research and Genome Canada)

### Materials and Methods

### Study Materials:

All NSCLC samples used in this study were excised from patients who were enrolled in a prospective, randomized controlled trial (JBR10) which studied the efficacy of adjuvant venorelbine plus cisplatin to improve survival in early stage (stage IB or II) NSCLC patients who had been treated by complete surgical resection (Winton et al., 2006). Half of the patients were randomly assigned to receive adjuvant chemotherapy, and half were assigned to no adjuvant chemotherapy. The samples examined were excised prior to any adjuvant therapy being administered. The study concluded that adjuvant chemotherapy prolongs disease free survival and overall survival in patients with completely resected early-stage NSCLC.

For array-comparative genomic hybridization (CGH) analysis, DNA was extracted from 134 formalin-fixed, paraffin-embedded (FFPE) and 16 fresh frozen NSCLC specimens, from 142 patients. The FFPE samples were cored from tissue blocks in areas of >60% tumour cells, as marked by a pathologist on hematoxylin and eosin (H&E) slides.

For gene expression microarray experiments, 176 fresh frozen tumour samples and 10 fresh frozen corresponding normal lung samples were used. 133 of these tumour samples were from patients in the JBR10 cohort, 81 of which also had array-CGH data analyzed in this study. 38 of the tumour samples were from a non-JBR10 cohort.

Array-CGH hybridization:

Array comparative genomic hybridization (CGH) was performed using a custom whole genome tiling path bacterial artificial chromosome array with 26,363 overlapping clones, each spotted in duplicate (BC Cancer Research Centre, Vancouver, BC) (Watson S K et al., 2007). This platform enables us to measure alterations in DNA copy number at high resolution across the entire genome in each tumour sample, with a minimal amount (as little as 50 ng) of DNA.

Comparative genomic hybridization experiments were undertaken as previously described (Coe & Lockwood et al., 2006). Briefly, each tumour DNA sample was labeled with Cyanine-3, mixed with a Cyanine-5-labeled individual male reference DNA sample, and hybridized to the array.

### Array-CGH Data Preprocessing and Normalization:

Array image capture and data normalization was performed as previously described (Watson S K et al., 2007). Briefly, post-hybridization arrays were scanned using a CCD-based imaging system, and quantitated using Soft-Worx Tracker spot analysis software (Applied Precision, Issaquah, Wash.).

Data was log 2 transformed, and replicate clones having standard deviations >0.075 or signal-to-noise ratios in each dye channel of <3 were filtered out. A multi-step normalization was then carried out to control for biases caused by the array (ex. spatial biases or differences in background signal), the dyes used for labeling, or the DNA sample quality (Khojasteh et al. 2005, Chi et al. 2007). The amount of “copycat” correction required for each sample was plotted in a histogram of all samples; those that required too much correction and did not lie within a normal distribution were deemed to be poor quality DNA, and were eliminated from analysis. By this criteria, 35 samples were eliminated, leaving 115 samples from 113 patients (56 received adjuvant chemotherapy, 57 had no adjuvant chemotherapy) for further analysis. Log 2 ratios were plotted and data was visualized using SeeGH software (Chi et al. 2004).

### Array-CGH Data Analysis:

In order to define genomic regions that were frequently gained in terms of DNA copy number in NSCLC, three algorithms were employed in parallel analyses to define the segmental DNA gains and losses in each tumour genome for the 113 patient samples: circular binary segmentation (DNAcopy) (Venkatraman & Olshen, 2007), a hidden markov model (HMMeR) (Shah et al., 2006), and aCGH Smooth (Jong et al. 2004). For DNAcopy analysis, a log 2 threshold of 0.05 for gains and −0.05 for losses was used to define whether a segment was gained/lost or not. For each algorithm, minimal common regions (MCRs) of DNA gain and loss were then identified for the entire tumor panel with STAC software (Diskin et al. 2006) (using 100 permutations at a resolution of 100,000 bp, and a p-value cut-off of 0.05 by either footprint or frequency calculation by the software). These regions are referred to herein and accompanying tables as “wide MCRs of gain” and “wide MCRs of loss.”

To attempt to focus further the genomic regions of DNA copy number gain in NSCLC, circular binary segmentation (DNAcopy) (Venkatraman & Olshen, 2007) was used to define the segmental DNA gains and losses in each tumour genome for the 113 patient samples. A log 2 threshold of 0.05 for gains and −0.05 for losses was used to define whether a segment was gained/lost or not. Minimal common regions (MCRs) of DNA gain and loss were then identified for the entire tumor panel with STAC software (Diskin et al. 2006) (using 100 permutations at a resolution of 100,000 bp) with a p-value cut-off of 0.05 by frequency calculation. MCRs corresponding to known copy number variations as described by Wong et al. 2007 were eliminated. As well, MCRs whose frequency of alteration amongst the samples multiplied by their average log 2 of altered samples was less than 0.02 were removed from further analysis. These MCRs are referred to as “narrow MCRs of gain” and “narrow MCRs of loss” herein.

In order to focus the array-CGH analysis on high-level amplification events in NSCLC, circular binary segmentation (DNAcopy) (Venkatraman & Olshen, 2007 was used to define the segmental DNA gains and losses in each tumour genome for the 113 patient samples. A log 2 threshold of 0.05 was used to define whether a segment was gained or not. High-amplitude regions of gain (referred to as “high-amplitude MCRs” herein) were defined as genomic regions where the average log 2 value, as assigned by DNAcopy analysis, in the gained samples, was greater than 0.15.

Prognostic and predictive genes by RNA expression levels within MCRs of gain were determined by integrating data from gene expression microarray experiments. Gene expression for 133 NSCLC samples was assessed using an Affymetrix U133A microarray chip. The data was normalized using RMAexpress software followed by distance-weighted discrimination (DWD) to minimize “batch” differences among samples, and then log 2 transformed.

### Statistical Analysis:

In order to identify prognostic genes, the MCRs of gain and loss as defined above (p-value 0.05 by frequency or footprint calculation) were cross-referenced with the locations of genes on the Affymetrix U133A chip (˜22,000 probesets in total) that were found to have prognostic value by univariate Cox proportional hazards analysis on the observation arm only. Out of 1584 probesets that had a significant prognostic effect (p<0.05) by gene expression, 398 probesets (364 genes) fell within MCRs of gain, and 426 probesets (391 genes) fell within MCRs of loss. These genes were selected for further analysis.

To evaluate the prognostic significance of genomic gain or loss at each of the genes in the absence of adjuvant therapy, a univariate Cox proportional hazards model using disease-specific survival (DSS) was applied to determine any statistically significant (p<0.05) prognostic effect for the patients who did not receive chemotherapy (57 patients). Hazard ratios were compared to ensure agreement between the gene expression and array-CGH data in terms of the effect on patient survival, and 4 lists of genes were arrived at: genes imparting a worse prognosis when gained (39 genes), genes imparting a better prognosis when gained (1 gene), genes imparting a worse prognosis when lost (13 genes), and genes imparting a better prognosis when lost (8 genes).

In addition, a univariate Cox proportional hazards model was employed on the entire cohort (observation and chemotherapy arm, 113 patients in total) with the use of interaction terms to identify effects of chemotherapy on the survival associated with gain or loss at each gene.

Genes within MCRs that were differentially expressed between tumours and normal lung samples were identified through significance analysis of microarray (SAM) analysis of the Affymetrix U133A expression microarray data from 176 NSCLC samples and 10 corresponding normal lung samples. The SAM parameters were as follows: FDR 5%, fold-change required 0.

To examine any clinicopathological associations between genomic gains and losses at each MCR, a Fisher's exact test was employed, using sex, nodal status, and histologic cell type as variables.

Quantitative Polymerase Chain Reaction (qPCR):

Quantitative PCR was performed using the SYBR Green method and the Roche Lightcycler 480 instrument. Five ng of genomic DNA were used per well in triplicate in 384 well plates. Primers were designed and tested for specificity using the online Primer Blast software (NCBI). Primers were designed to target one exon region of each gene, with a bias towards 3′ exon location. As a reference, primers were designed for 3 genes on different chromosomes that are infrequently altered numerically in NSCLC, as guided by our array-CGH results. Dissociation curves (melting curves) for each primer pair were determined to test for contamination, mispriming, and primer-dimer artifact; only primers producing a single peak in the dissociation curve were used in the assays.

Standard curves were derived using pooled DNA from 20 formalin-fixed paraffin-embedded lung tissue from resection specimens, taken from blocks uninvolved by tumour. In addition, 23 normal FFPE lung samples were run along with the tumour samples in each reaction.

Initial processing of data was carried out using the Roche Lightcycler 480 software, which calculates using the 2^{nd} derivative max point to determine crossing-point (CP) values for each well. CP values were mapped to the standard curve for each gene to obtain DNA concentration values for each well. The gene copy number was normalized against the copy number of the reference genes. A normal range of gene copy number for each gene was established with the 23 samples of non-neoplastic lung DNA, and samples with copy number 2sd above the mean were identified as gained in copy number. Samples with copy numbers, as calculated by advanced relative quantification, of greater than 4, were identified as having an amplification (in addition to a gain) at that gene, by qPCR analysis.

### Example 2

### Selection of Genes for Quantitative PCR Validation

Genes within wide MCRs of gain on chromosomes 5, 8, and 12 that showed concordant survival effect by transcript level and DNA copy number were chosen for the first round of quantitative PCR validation.

For the second round of quantitative PCR validation, 5 genes within each prognostic/predictive high-amplitude MCR were selected by ranking them using the following criteria: RNA expression data showing the same survival effect for the RNA transcript quantity as for the DNA copy number, gene ontology relating to oncogenicity, average log 2 (“raw” log 2 values as well as log 2 values assigned by DNAcopy) among gained samples, STAC analysis frequency p-value<0.05, overexpression of RNA transcripts in NSCLC, location within an amplicon reported previously in the literature, p-values of prognostic and predictive survival associations for DNA copy number at that location (both univariate and multivariate), and p-values for prognostic and predictive survival associations of RNA transcript levels (univariate).

### Example 3

The array-CGH dataset described in Example 1 is unique and powerful in that it uses tumour samples from a randomized controlled trial of the effectiveness of chemotherapy in early-stage NSCLC, providing an unprecedented opportunity to study genomic aberrations at high-resolution and correlating them with patient outcome in the presence or absence of chemotherapy. The sample size (113) is more than double the majority of previous array-CGH studies, allowing for a greater power in determining prognostic and predictive effects of gains and losses. Furthermore, the resolution of our platform is superior to most previous array-CGH studies in NSCLC, allowing us to more precisely define the breakpoints of amplifications and deletions. An additional 180 samples from the same trial will be processed to further validate the survival associations found in the array-CGH study described herein.

### Example 4

### Optimization of the Prognostic and Predictive Gene Copy Number Model

The gains and losses outlined herein could be tested for associations amongst one another using methods of multivariate statistics including but not limited to, cluster analysis, principal component analysis, and logistic regression. In this way, copy number alterations that tend to occur together could be identified, and key alterations that could serve as surrogate biomarkers for the co-occurring events could be identified. These key copy number alterations could be incorporated into a weighted model or that could be used to identify one or more “copy number signatures” that could molecularly classify non-small cell lung carcinomas. Such a signature would be useful for predicting prognosis and response to chemotherapy.

### Example 5

The sample of lung tumour is obtained during surgery or a minimally invasive procedure. The tissue is processed in the lab to identify the tumour content. A portion of the tumour is frozen, or fixed in formalin and embedded in paraffin as per standard laboratory protocol. The DNA is extracted from the tumour tissue, and subjected to a laboratory test to examine for specific genomic alterations, such as array-CGH or multiplex qPCR. Alternatively, sections are cut from a paraffin block containing tumour, and processed for FISH analysis using probes hybridizing to one or more of our targets, and the tumour nuclei are scored for gains and losses. The presence as determined by these tests of a gain or loss in copy number, compared to a control (internal or external, depending on the test), indicates a poor prognosis for the patient if not treated with chemotherapy, but a significantly improved prognosis if treated with chemotherapy.

### Example 6

How to identify probes used herein useful for detecting gains and losses associated with prognosis.

An individual could take the known genomic location of the MCR and then apply online resources to determine which BAC clones span the recurring alteration (e.g. Human BAC Resource—http://www.ncbi.nlm.nih.gov/genome/cyto/hbrc.shtml). SMRT array mapping information—specific to individual BAC clones—is available online (http://www.bccrc.ca/cg/ArrayCGH_Group.html, http://bacpac.chori.org/order.php).

Individuals can take the known genomic location, open the mapping file, and determine which BAC clones span the MCR region they are interested in. Individuals could then order clone(s) for their own use from an online resource (e.g. BACPAC Resources Center http://bacpac.chori.org/order.php). Labeled probes from this DNA could then be made and applied using a standard FISH protocol. Alternatively, labeled probes for FISH from a given clone could also be ordered directly from a variety of sources, including the BC Cancer Research Centre (http://arraycgh.ca/services.php).

### Tables

TABLE 1
Narrow Minimal Common Regions (MCRs) of Gain Associated with Prognosis by array-CGH analysis

| | | | | Proportion | | | # genes | # genes with |
|---|---|---|---|---|---|---|---|---|
| | | | MCR | of | Poor survival in | Improved survival | tested | same survival |
| | BP start | BP end | length | Tumours | absence of | with | by | association by |
| Chromosome | position* | position* | (Mb) | with Gain | chemotherapy? | chemotherapy? | qPCR | qPCR |
| 8 | 133600000 | 135300000 | 1.7 | 0.50 | No effect on survival | Yes (p = 0.011) | 2 | 0 significant |
| | | | | | | | | 2 trending |
| 11 | 68500000 | 71000000 | 2.5 | 0.22 | Trend to yes | Yes (p = 0.056) | 1 | 1 |
| | | | | | (p = 0.17) | (multivariate | | |
| | | | | | | p = 0.02) | | |
| 12 | 51000000 | 53400000 | 2.4 | 0.25 | Yes (p < 0.001, | Yes (p = 0.004) | 10 | 4 significant |
| | | | | | q = 0.011) | (multivariate | | 1 trending |
| | | | | | | p = 0.003) | | |
| 12 | 54200000 | 54800000 | 0.6 | 0.22 | Yes (p < 0.001, | Trend to yes | 5 | 0 significant |
| | | | | | q = 0.007) | (p = 0.056) | | 2 trending |
| 12 | 54900000 | 55200000 | 0.3 | 0.22 | Yes (p < 0.001, | Trend to yes | 0 | NA |
| | | | | | q = 0.007) | (p = 0.163) | | |
| 12 | 55600000 | 55700000 | 0.1 | 0.22 | Yes (p < 0.001, | Trend to yes | 1 | 0 |
| | | | | | q = 0.001) | (p = 0.156) | | |
| 12 | 56400000 | 56700000 | 0.3 | 0.23 | Yes (p < 0.001, q < | Trend to yes | 4 | 3 |
| | | | | | 0.001) | (p = 0.119) | | |

TABLE 2
Wide MCRs of gain associated with prognosis by array-CGH analysis

| | | | | | | | # | |
|---|---|---|---|---|---|---|---|---|
| | | | | | | | genes | # genes with |
| | | | MCR | Proportion | Poor survival | Improved survival | tested | same survival |
| | BP start | BP end | Length | of Samples | in absence of | with | by | association by |
| Chromosome | position* | position* | (Mb) | with Gain | chemotherapy? | Chemotherapy? | qPCR | qPCR |
| 1 | 21700000 | 22800000 | 1.1 | 0.21 | No | Yes (p = 0.023) | 0 | NA |
| 1 | 27000000 | 27400000 | 0.4 | 0.22 | No | Yes (p = 0.026) | 0 | NA |
| 1 | 36600000 | 37200000 | 0.6 | 0.20 | No | Yes (p = 0.005) | 0 | NA |
| 1 | 43000000 | 43200000 | 0.2 | 0.21 | No | Yes (p = 0.002) | 3 | 0 significant |
| | | | | | | | | 3 trending |
| 1 | 43200000 | 43300000 | 0.1 | 0.19 | Yes (p = 0.031) | Yes (p = 0.003) | 0 | NA |
| 1 | 43400000 | 44100000 | 0.7 | 0.21 | No | Yes (p = 0.007) | 0 | NA |
| 2 | 222500000 | 222600000 | 0.1 | 0.06 | Yes (p < 0.001) | Yes (p = 0.004) | 0 | NA |
| 4 | 59100000 | 59300000 | 0.2 | 0.07 | Trend to yes | Yes (p = 0.018) | 0 | NA |
| | | | | | (p = 0.068) | | | |
| 5 | 1 | 44600000 | 44.6 | 0.57 | Yes (p = 0.018) | Yes (p = 0.007) | 7 | 0 |
| 5 | 45400000 | 45900000 | 0.5 | 0.32 | Yes (p = 0.013) | No | 0 | NA |
| 5 | 49400000 | 52900000 | 3.5 | 0.16 | Yes (p = 0.003) | No | 0 | NA |
| 6 | 61900000 | 72800000 | 10.9 | 0.20 | Yes (p = 0.011) | No | 0 | NA |
| 8 | 90700000 | 146100000 | 55.4 | 0.57 | No | Yes (p = 0.004) | 9 | 2 significant |
| | | | | | | | | 5 trending |
| 8 | 102000000 | 104200000 | 2.2 | 0.44 | Trend to yes | Yes (p < 0.001) | 0 | NA |
| | | | | | (p = 0.083) | | | |
| 8 | 118700000 | 120300000 | 1.6 | 0.39 | No | Yes (p = 0.003) | 0 | NA |
| 8 | 123400000 | 138100000 | 14.7 | 0.58 | No | Yes (p = 0.001) | 6 | 1 significant |
| | | | | | | | | 4 trending |
| 8 | 139400000 | 139500000 | 0.1 | 0.37 | No | Yes (p = 0.043) | 0 | NA |
| 9 | 35500000 | 38200000 | 2.7 | 0.12 | No | Yes (p = 0.018) | 0 | NA |
| 12 | 36900000 | 37000000 | 0.1 | 0.06 | Yes (p = 0.026) | Trend to yes | 0 | NA |
| | | | | | | (p = 0.082) | | |
| 12 | 46200000 | 55500000 | 9.3 | 0.28 | Yes (p = 0.005) | Yes (p = 0.047) | 19 | 6 significant |
| | | | | | | | | 2 trending |
| 12 | 55600000 | 56500000 | 0.9 | 0.22 | Yes (p < 0.001) | No | 10 | 5 significant |
| 12 | 58700000 | 59200000 | 0.5 | 0.23 | Yes (p = 0.007) | No | 0 | NA |
| 14 | 18000000 | 23400000 | 5.4 | 0.44 | Yes (p = 0.043) | No | 0 | NA |
| 14 | 41300000 | 42200000 | 0.9 | 0.25 | Yes (p = 0.046) | No | 0 | NA |
| 16 | 44900000 | 45100000 | 0.2 | 0.11 | Yes (p = 0.046) | No | 0 | NA |
| 19 | 8600000 | 8800000 | 0.2 | 0.13 | Yes (p = 0.041) | Yes (p = 0.007) | 0 | NA |

TABLE 3
Wide MCRs of loss associated with poor prognosis and/or significant
response to chemotherapy by array-CGH analysis

| | | | MCR | Proportion | Poor survival | Improved |
|---|---|---|---|---|---|---|
| | BP start | BP end | Size | of samples | in absence of | survival with |
| Chromosome | position* | position* | (Mb) | with loss | chemotherapy? | chemotherapy? |
| 1 | 107600000 | 121000000 | 13.4 | 0.29 | Yes (p = 0.014) | Yes (p = 0.020) |
| 1 | 241100000 | 241300000 | 0.2 | 0.10 | No | Yes (p = 0.021) |
| 1 | 243200000 | 243800000 | 0.6 | 0.12 | No | Yes (p = 0.01) |
| 3 | 1 | 17900000 | 17.9 | 0.43 | No | Yes (p = 0.014) |
| 3 | 36300000 | 73900000 | 37.6 | 0.43 | No | Yes (p = 0.030) |
| 3 | 193100000 | 194300000 | 1.2 | 0.10 | No | Yes (p = 0.025) |
| 5 | 61600000 | 68700000 | 7.1 | 0.35 | Trend to yes | Yes (p = 0.028) |
| | | | | | (p = 0.087) | |
| 5 | 70800000 | 74500000 | 3.7 | 0.36 | Yes (p = 0.036) | Yes (p = 0.047) |
| 5 | 75900000 | 77600000 | 1.7 | 0.32 | No | Yes (p = 0.050) |
| 5 | 166900000 | 180600000 | 13.7 | 0.40 | No | Yes (p = 0.023) |
| 8 | 56200000 | 56600000 | 0.4 | 0.13 | Yes (p = 0.025) | No |
| 11 | 1 | 3500000 | 3.5 | 0.27 | No | Yes (p = 0.010) |
| 11 | 3700000 | 3800000 | 0.1 | 0.24 | No | Yes (p = 0.040) |
| 12 | 113100000 | 129500000 | 16.4 | 0.26 | No | Yes (p = 0.037) |
| 13 | 84300000 | 90000000 | 5.7 | 0.27 | Trend to yes | Yes (p = 0.04) |
| | | | | | (p = 0.062) | |
| 18 | 69400000 | 76000000 | 6.6 | 0.35 | No | Yes (p = 0.029) |

TABLE 4
Wide MCRs of loss associated with
good prognosis by array-CGH analysis

| | | | | Proportion | Good |
|---|---|---|---|---|---|
| | | | MCR | of | prognosis in |
| Chro- | BP start | BP end | Size | samples | absence of |
| mosome | position* | position* | (Mb) | with loss | chemotherapy? |
| 2 | 85600000 | 91700000 | 6.1 | 0.22 | Yes (p = 0.032) |
| 2 | 94600000 | 95900000 | 1.3 | 0.19 | Yes (p = 0.047) |
| 6 | 2200000 | 6500000 | 4.3 | 0.16 | Yes (p = 0.028) |
| 9 | 36400000 | 46200000 | 9.8 | 0.51 | Yes (p = 0.030) |
| 14 | 18100000 | 18700000 | 0.6 | 0.16 | Yes (p = 0.044) |

TABLE 5
Poor prognosis genes when gained as determined by aCGH and RNA microarray analysis

| | | | | | | | | Significant |
|---|---|---|---|---|---|---|---|---|
| | | | | | | Proportion | | improvement |
| | | | | | | of | | with |
| | Entrez | | BP start | BP end | | samples | p- | chemotherapy? |
| Gene_Symbol | Gene ID | Chromosome | position* | position* | strand | gained | value | (p < 0.05) |
| MFSD7 | 84179 | 4 | 665618 | 672973 | − | 0.09 | 0.0026 | yes |
| D4S234E | 27065 | 4 | 4438884 | 4471686 | + | 0.1 | 0.0397 | yes |
| ACOX3 | 8310 | 4 | 8418909 | 8493352 | − | 0.12 | 0.0174 | yes |
| SRD5A1 | 6715 | 5 | 6686500 | 6722675 | + | 0.48 | 0.0365 | yes |
| ADCY2 | 108 | 5 | 7449343 | 7883194 | + | 0.45 | 0.0202 | no |
| clone Z146 | none | 5 | 10594566 | 10596305 | + | 0.46 | 0.013 | no |
| | (unigene ID | | | | | | | |
| | Hs.544229) | | | | | | | |
| ANKH | 56172 | 5 | 14762019 | 14924876 | − | 0.45 | 0.0434 | no |
| CDH18 | 1016 | 5 | 19508898 | 20017046 | − | 0.47 | 0.0481 | no |
| OXCT1 | 5019 | 5 | 41765924 | 41906548 | − | 0.37 | 0.0031 | no |
| UTRN | 7402 | 6 | 144654566 | 145215863 | + | 0.06 | 0.0089 | no |
| cDNA | none | 7 | 50485828 | 50488511 | + | 0.23 | 0.0234 | no |
| DKFZp434E2423 | (unigene ID | | | | | | | |
| | Hs.244772) | | | | | | | |
| C9orf68 | 55064 | 9 | 4588316 | 4656464 | − | 0.06 | 0.011 | no |
| AQP2 | 359 | 12 | 48630796 | 48638931 | + | 0.18 | 0.0257 | yes |
| ACCN2 | 41 | 12 | 48737754 | 48763661 | + | 0.18 | 0.0257 | yes |
| SLC11A2 | 4891 | 12 | 49666044 | 49706409 | − | 0.18 | 0.0257 | yes |
| SCN8A | 6334 | 12 | 50271287 | 50488574 | + | 0.18 | 0.0257 | yes |
| KRT81 | 3887 | 12 | 50965964 | 50971566 | − | 0.21 | 0.0079 | yes |
| KRT1 | 3848 | 12 | 51354787 | 51360458 | − | 0.26 | 0.0098 | yes |
| ESPL1 | 9700 | 12 | 51948350 | 51973694 | + | 0.24 | 0.0051 | yes |
| NPFF | 8620 | 12 | 52186741 | 52187689 | − | 0.23 | 0.0051 | yes |
| ATP5G2 | 517 | 12 | 52345211 | 52356779 | − | 0.22 | 0.0088 | yes |
| HOXC11 | 3227 | 12 | 52653177 | 52656470 | + | 0.22 | 0.0125 | yes |
| NEUROD4 | 58158 | 12 | 53699996 | 53710068 | + | 0.19 | 0.001 | yes |
| ITGA7 | 3679 | 12 | 54364619 | 54387894 | − | 0.18 | 0.0022 | no |
| CDK2/BCDO2 | 1017/ | 12 | 54646826 | 54652836 | + | 0.18 | 0.0079 | no |
| | 83875 | | | | | | | |
| ERBB3 | 2065 | 12 | 54760159 | 54783395 | + | 0.19 | 0.0079 | no |
| DLST/PA2G4 | 1743/ | 12 | 54784628 | 54793913 | + | 0.19 | 0.0079 | no |
| | 389424 | | | | | | | |
| PRIM1 | 5557 | 12 | 55411631 | 55432413 | − | 0.16 | 0.0024 | no |
| ZBTB39 | 9880 | 12 | 55678885 | 55686497 | − | 0.19 | 0.0003 | yes |
| KIAA0286 | 23306 | 12 | 55735693 | 55758813 | − | 0.19 | 0.0003 | yes |
| INHBE | 83729 | 12 | 56135363 | 56138058 | + | 0.19 | 0.0011 | yes |
| MARS | 4141 | 12 | 56168118 | 56196700 | + | 0.19 | 0.0011 | yes |
| B4GALNT1 | 2583 | 12 | 56305818 | 56313252 | − | 0.19 | 0.0011 | yes |
| TSFM | 10102 | 12 | 56462826 | 56476784 | + | 0.19 | 0.0043 | yes |
| TRHDE | 29953 | 12 | 70952730 | 71345689 | + | 0.11 | 0.0214 | no |
| OR1E1/OR1E2 | 8387/ | 17 | 3282914 | 3283886 | − | 0.11 | 0.0358 | no |
| | 8388 | | | | | | | |
| RCVRN | 5957 | 17 | 9741752 | 9749409 | − | 0.05 | 0.0032 | no |
| DNMT3B | 1789 | 20 | 30813852 | 30860823 | + | 0.27 | 0.0401 | yes |

TABLE 6
Good prognosis genes when gained as determined by aCGH and RNA microarray analysis

| | | | | | | Proportion | |
|---|---|---|---|---|---|---|---|
| | | | | | | of | |
| | Entrezgene | | BP start | BP end | | samples | p- |
| Gene_Symbol | ID | Chromosome | position* | position* | strand | gained | value |
| RAB11FIP1 | 80223 | 8 | 37835628 | 37876161 | − | 0.19 | 0.017 |

TABLE 7
Poor prognosis genes when lost as determined by aCGH and RNA microarray analysis

| | | | | | | Proportion | | Significant |
|---|---|---|---|---|---|---|---|---|
| | | | | | | of | | improvement |
| | Entrez | | | | | samples | | with |
| Gene | Gene | | BP start | BP end | | with DNA | p- | chemotherapy? |
| Symbol | ID | Chromosome | position* | position* | strand | loss | value | (p < 0.05) |
| AHCYL1 | 10768 | 1 | 110328831 | 110367887 | + | 0.31 | 0.028 | no |
| RHOC | 389 | 1 | 113045272 | 113051548 | − | 0.32 | 0.013 | yes |
| ATP1A1 | 476 | 1 | 116717359 | 116748919 | + | 0.31 | 0.023 | no |
| IGSF3 | 3321 | 1 | 116918554 | 117011837 | − | 0.31 | 0.023 | no |
| ELF1 | 1997 | 13 | 40404164 | 40454418 | − | 0.33 | 0.046 | no |
| RGC32 | 28984 | 13 | 40929542 | 40943013 | + | 0.35 | 0.046 | no |
| ESD | 2098 | 13 | 46243392 | 46269368 | − | 0.36 | 0.046 | no |
| TAF1C | 9013 | 16 | 82768962 | 82778163 | − | 0.26 | 0.032 | no |
| ATP2C2 | 9914 | 16 | 82959634 | 83055294 | + | 0.26 | 0.032 | yes |
| ZDHHC7 | 55625 | 16 | 83565573 | 83602642 | − | 0.26 | 0.024 | yes |
| COX4I1 | 1327 | 16 | 84390697 | 84398109 | + | 0.26 | 0.024 | yes |
| FOXF1 | 2294 | 16 | 85101634 | 85105571 | + | 0.26 | 0.024 | yes |
| MAP1LC3B | 81631 | 16 | 85983320 | 85995881 | + | 0.27 | 0.05 | no |

TABLE 8
Good prognosis genes when lost as determined by aCGH and RNA microarray analysis

| | | | | | | Proportion | |
|---|---|---|---|---|---|---|---|
| | | | | | | of | |
| | | | | | | samples | |
| | | | BP start | BP end | | with DNA | p- |
| Gene Symbol | Entrez Gene ID | Chromosome | position* | position* | strand | loss | value |
| CDYL | 9425 | 6 | 4651392 | 4900777 | + | 0.16 | 0.028 |
| C6orf15 | 29113 | 6 | 31186979 | 31188311 | − | 0.15 | 0.039 |
| NCR3 | 259197 | 6 | 31664651 | 31668741 | − | 0.15 | 0.039 |
| MSH5/ | 401251/4439 | 6 | 31815753 | 31840606 | + | 0.15 | 0.039 |
| C6orf26 | | | | | | | |
| HLA-DOA | 3111 | 6 | 33079937 | 33085367 | − | 0.16 | 0.028 |
| RXRB | 6257 | 6 | 33269343 | 33276410 | − | 0.16 | 0.028 |
| KIFC1 | 3833 | 6 | 33467583 | 33485625 | + | 0.16 | 0.028 |
| TCL6 | 27004 | 14 | 95187268 | 95215923 | + | 0.19 | 0.187 |

All basepair positions in the tables refer to positions on the NCBI human genome build 36.3.

TABLE 9
Survival associations of genes within wide MCRs as determined by qPCR analysis

| | | | | Significant | | |
|---|---|---|---|---|---|---|
| | | | | poor | Significant | Amplitude of |
| | | | | prognosis in | improved | copy number |
| Gene | | BP start | BP end | observation | response to | associated |
| Symbol | Chromosome | position | position | arm? | chemotherapy? | with survival |
| TERT | 5 | 1306286 | 1348162 | no | no | NA |
| BC035019 | 5 | 3470265 | 3589161 | no | no | NA |
| SRD5A1 | 5 | 6686499 | 6722675 | no | no | NA |
| ADCY2 | 5 | 7846731 | 7883194 | no | no | NA |
| ANKH | 5 | 14762018 | 14799111 | no | no | NA |
| CDH18 | 5 | 19508897 | 20017044 | no | no | NA |
| OXCT1 | 5 | 41765923 | 41906548 | no | no | NA |
| RAB11FIP1 | 8 | 37852535 | 38058325 | no | no | NA |
| BAALC | 8 | 104222096 | 104311709 | no | yes | gain |
| | | | | | (p 0.047, HR | |
| | | | | | 0.17) | |
| ANGPT1 | 8 | 108330885 | 108579430 | trend | trend | gain |
| | | | | (p 0.079, HR | (p 0.077, HR | |
| | | | | 2.70) | 0.21) | |
| MAL2 | 8 | 120289790 | 120327092 | no | no | NA |
| MYC | 8 | 128784030 | 128957168 | no | yes | amp |
| | | | | | (p 0.010, HR 0) | |
| WISP1 | 8 | 134272493 | 134310753 | no | trend | gain |
| | | | | | (p 0.078, HR 0) | |
| NDRG1 | 8 | 134318595 | 134337653 | no | trend | gain |
| | | | | | (p 0.139, HR 0) | |
| AQP2 | 12 | 48630795 | 48638931 | no | no | NA |
| ACCN2 | 12 | 48737753 | 48763661 | no | no | NA |
| SLC11A2 | 12 | 49666041 | 49706423 | no | no | NA |
| SCN8A | 12 | 50271286 | 50488566 | no | no | NA |
| KRT81 | 12 | 50965963 | 50988422 | no | yes | amp |
| | | | | | (p 0.021, HR 0) | |
| KRT1 | 12 | 51354718 | 51360458 | no | yes | continuous |
| | | | | | (p 0.047, HR | |
| | | | | | 0.06) | |
| ESPL1 | 12 | 51948383 | 51973694 | no | no | NA |
| MAP3K12 | 12 | 52160546 | 52179538 | no | no | NA |
| NPFF | 12 | 52188225 | 52187689 | no | no | NA |
| ATP5G2 | 12 | 52345252 | 52356779 | no | no | NA |
| HOXC11 | 12 | 52653176 | 52656470 | yes | no | gain |
| | | | | (p 0.007, HR | | |
| | | | | 4.54) | | |
| NEUROD4 | 12 | 53706622 | 53707486 | no | yes | gain |
| | | | | | (p 0.037, HR | |
| | | | | | 0.09) | |
| ITGA7 | 12 | 54364618 | 54387894 | trend | no | gain |
| | | | | (p 0.092, HR | | |
| | | | | 2.72) | | |
| CDK1 | 12 | 54646825 | 54652835 | no | no | NA |
| ERBB3 | 12 | 54760158 | 54783395 | no | no | NA |
| PA2G4 | 12 | 54784627 | 54793961 | no | trend | continuous |
| | | | | | (p 0.099, HR | |
| | | | | | 0.10) | |
| DLST | 12 | 54784627 | 54793961 | no | no | NA |
| PRIM1 | 12 | 55411312 | 55432413 | yes | no | amp |
| | | | | (p 0.024, HR | | |
| | | | | 8.24) | | |
| ZBTB39 | 12 | 55678884 | 55686497 | no | no | NA |
| KIAA0286 | 12 | 55735697 | 55758810 | no | no | NA |
| INHBE | 12 | 56135378 | 56138058 | no | no | NA |
| MARS | 12 | 56167343 | 56197601 | no | no | NA |
| B4GALNT1 | 12 | 56303459 | 56313252 | yes | no | amp |
| | | | | (p 0.024, HR | | |
| | | | | 3.70) | | |
| OS9 | 12 | 56374152 | 56401607 | yes | no | amp |
| | | | | (p 0.008, HR | | |
| | | | | 3.17) | | |
| CDK4 | 12 | 56428269 | 56432431 | yes | no | amp |
| | | | | (p 0.022, HR | | |
| | | | | 3.60) | | |
| TSFM | 12 | 56462850 | 56476784 | yes | no | amp |
| | | | | (p 0.011, HR | | |
| | | | | 10.49) | | |

HR refers to Hazard ratio.
In the column relating to improvement with chemotherapy, a HR of 0 indicates that no subjects in the chemotherapy-treated group died due to disease. A HR of 0.1 means that the risk of dying due to disease was 10 times greater in the non-chemotherapy-treated group compared to the chemotherapy-treated group.
A gene identified as “amp” is a higher threshold gain than a gene identified as a “gain” (e.g. an “amp” gene comprises a gain of greater than 4 copies by qPCR analysis.
A gene identified as “continuous” refers to a gene that shows an increasing survival effect with increasing amplitude of DNA copy gain or amplification, by cox proportional hazards statistical analysis on continuous copy number data..

TABLE 10
High-amplitude MCRs with survival associations by array-CGH data analysis

| | | | | | | | | | | # genes |
|---|---|---|---|---|---|---|---|---|---|---|
| | | | | | | | Prognostic | Predictive of | # genes | with same |
| | | | | MCR | Gain | Amplification | effect in | improved | tested | survival |
| MCR | | | | size | frequency | frequency | observation | response to | by | association |
| ID | Chromoosome | BP start | BP end | (Mb) | (%) | (%) | arm? | chemotherapy? | qPCR | by qPCR |
| NRG-4 | 1 | 41265460 | 43221579 | 2.0 | 20 | 5 | None | yes | 5 | 0 significant |
| | | | | | | | | (p 0.003, HR 0) | | 5 trending |
| NRG- | 2 | 61986306 | 63127125 | 1.1 | 16 | 4 | poor | yes | 3 | 0 |
| 11 | | | | | | | (p 0.002, HR | (p 0.01, HR | | |
| | | | | | | | 3.40)* | 0.10)* | | |
| NRG- | 8 | 36761058 | 38829703 | 2.1 | 25 | 11 | good | no | 3 | 0 |
| 56 | | | | | | | (p 0.042, HR | | | |
| | | | | | | | 0.25) | | | |
| NRG- | 8 | 128289292 | 128936748 | 0.6 | 54 | 13 | None | yes | 4 | 1 significant |
| 58 | | | | | | | | (p 0.018, HR | | 2 trending |
| | | | | | | | | 0.26)* | | |
| NRG- | 11 | 68572940 | 70388868 | 1.8 | 22 | 7 | None | trend to yes | 1 | 1 |
| 74 | | | | | | | | (p 0.056, HR | | |
| | | | | | | | | 0.19) | | |
| NRG- | 12 | 50731457 | 51457372 | 0.7 | 24 | 4 | poor | yes | 2 | 2 |
| 79 | | | | | | | (p 0.008, HR | (p 0.039, HR | | |
| | | | | | | | 3.16) | 0.21) | | |
| NRG- | 12 | 52696908 | 53538441 | 0.8 | 23 | 3 | poor | yes | 1 | 1 |
| 80 | | | | | | | (p < 0.001, HR | (p 0.002, HR | | |
| | | | | | | | 4.70) | 0.09) | | |
| NRG- | 12 | 55933813 | 57461765 | 1.5 | 24 | 5 | poor | trend to yes | 8 | 5 |
| 81 | | | | | | | (p < 0.001, HR | (p 0.081, HR | | |
| | | | | | | | 6.81) | 0.29) | | |
| NRG- | 12 | 64438067 | 68503251 | 4.1 | 15 | 5 | poor | no | 5 | 4 |
| 82 | | | | | | | (p < 0.001, HR | | | |
| | | | | | | | 4.64) | | | |
| NRG- | 14 | 96994959 | 99058653 | 2.1 | 30 | 4 | poor | yes | 3 | 1 |
| 89 | | | | | | | (p 0.061, HR | (p 0.104, HR | | |
| | | | | | | | 2.26) | 0.29) | | |
| NRG- | 20 | 30409813 | 30901867 | 0.5 | 36 | 8 | none | yes | 3 | 0 |
| 119 | | | | | | | | (p 0.014, HR | | |
| | | | | | | | | 0.14) | | |

*Survival effect significant using amplitude of gain as a continuous variable

TABLE 11
Survival associations of genes within high-amplitude MCRs, by qPCR analysis

| | | | | | | | Amplitude |
|---|---|---|---|---|---|---|---|
| | | | | | Significant | | of copy |
| | | | | | poor | Significant | number |
| | | | | | prognosis in | improved | associated |
| Gene | Associated | | BP start | BP end | observation | response to | with |
| Symbol | MCR ID | Chromosome | position | position | arm? | chemotherapy? | survival |
| GUCA2A | NRG4 | 1 | 42400948 | 42402982 | trend to yes | trend to yes | gain |
| | | | | | (p 0.117, HR | (p 0.190, HR 0.29) | |
| | | | | | 2.41) | | |
| PPIH | NRG4 | 1 | 42896634 | 42915016 | no | trend to yes | gain |
| | | | | | | (p 0.139, HR 0.19) | |
| LEPRE1 | NRG4 | 1 | 42984631 | 43005270 | trend to yes | trend to yes | gain |
| | | | | | (p 0.089, HR | (p 0.172, HR 0.27) | |
| | | | | | 1.99)* | | |
| CR623026 | NRG4 | 1 | 43003707 | 43005283 | no | trend to yes | gain |
| | | | | | | (p 0.115, HR 0) | |
| C1orf50 | NRG4 | 1 | 43005526 | 43013998 | yes | trend to yes | gain |
| | | | | | (p 0.037, HR | (p 0.060, HR 0.17) | |
| | | | | | 3.15) | | |
| TMEM17 | NRG11 | 2 | 62581263 | 62586980 | no | no | NA |
| BC038779 | NRG11 | 2 | 62692510 | 62743267 | no | no | NA |
| EHBP1 | NRG11 | 2 | 62786636 | 63127125 | no | no | NA |
| RAB11FIP1 | NRG56 | 8 | 37835627 | 37849954 | no | no | NA |
| WHSC1L1 | NRG56 | 8 | 38251717 | 38256885 | no | no | NA |
| FGFR1 | NRG56 | 8 | 38387812 | 38445509 | no | no | NA |
| AK125310 | NRG58 | 8 | 128289292 | 128300515 | no | no | NA |
| DQ515898 | NRG58 | 8 | 128371243 | 128502801 | no | trend to yes | amplification |
| | | | | | | (p 0.061, HR | |
| | | | | | | 0.13) | |
| DQ515897 | NRG58 | 8 | 128371243 | 128563566 | no | trend to yes | amplification |
| | | | | | | (p 0.076, HR 0) | |
| MYC | NRG58 | 8 | 128784030 | 128957168 | no | yes | amplification |
| | | | | | | (p 0.010, HR 0) | |
| FGF3 | NRG74 | 11 | 69333916 | 69343129 | yes | yes | amplification |
| | | | | | (p 0.021, HR | (p 0.043, HR 0) | |
| | | | | | 3.08)* | | |
| KRT81 | NRG79 | 12 | 50965963 | 50988422 | no | yes | amplification |
| | | | | | | (p 0.021, HR 0) | |
| KRT1 | NRG79 | 12 | 51354718 | 51360458 | no | yes | continuous |
| | | | | | | (p 0.047, HR | |
| | | | | | | 0.06)* | |
| FAM112B | NRG80 | 12 | 53136010 | 53153614 | yes | yes | gain |
| | | | | | (p 0.019, HR | (p 0.040, HR 0) | |
| | | | | | 8.74) | | |
| INHBE | NRG81 | 12 | 56135378 | 56138058 | no | no | NA |
| MARS | NRG81 | 12 | 56167343 | 56197601 | no | no | NA |
| B4GALNT1 | NRG81 | 12 | 56303459 | 56313252 | yes | trend to yes | amplification |
| | | | | | (p 0.024, HR | (p 0.185, HR | |
| | | | | | 3.70) | 0.59) | |
| OS9 | NRG81 | 12 | 56374152 | 56401607 | yes | no | amplification |
| | | | | | (p 0.008, HR | | |
| | | | | | 3.17) | | |
| CENTG1 | NRG81 | 12 | 56405260 | 56418296 | yes | trend to yes | gain |
| | | | | | (p 0.025, HR | (p 0.117, HR | |
| | | | | | 3.41) | 0.25) | |
| TSPAN31 | NRG81 | 12 | 56425050 | 56428293 | no | no | NA |
| CDK4 | NRG81 | 12 | 56428269 | 56432431 | yes | no | amplification |
| | | | | | (p 0.022, HR | | |
| | | | | | 3.60) | | |
| TSFM | NRG81 | 12 | 56462850 | 56476784 | yes | no | amplification |
| | | | | | (p 0.011, HR | | |
| | | | | | 10.49) | | |
| DYRK2 | NRG82 | 12 | 66329020 | 66340410 | no | no | NA |
| AK024870 | NRG82 | 12 | 66340990 | 66344213 | yes | no | amplification |
| | | | | | (p 0.017, HR | (p 0.079, HR | |
| | | | | | 8.99) | 9.86)** | |
| NUP107 | NRG82 | 12 | 67366997 | 67422740 | yes | no | amplification |
| | | | | | (p 0.017, HR | | |
| | | | | | 8.99) | | |
| MDM2 | NRG82 | 12 | 67488246 | 67520481 | yes | no | amplification |
| | | | | | (p 0.024, HR | | |
| | | | | | 8.24) | | |
| CPSF6 | NRG82 | 12 | 67919666 | 67951290 | yes | no | amplification |
| | | | | | (p 0.017, HR | (p 0.074, HR | |
| | | | | | 8.99) | 20.74)** | |
| BC038465 | NRG89 | 14 | 96994959 | 97000249 | no | no | NA |
| AK097943 | NRG89 | 14 | 97469151 | 97514200 | no | no | NA |
| BCL11B | NRG89 | 14 | 98705377 | 98807575 | yes | trend to yes | gain |
| | | | | | (p 0.029, HR | (p 0.188, HR | |
| | | | | | 3.51) | 0.23) | |
| ASXH1 | NRG119 | 20 | 30409813 | 30479886 | trend to yes | no | gain |
| | | | | | (p 0.159, HR | | |
| | | | | | 2.22) | | |
| C20orf112 | NRG119 | 20 | 30498329 | 30534849 | trend to yes | no | gain |
| | | | | | (p 0.193, HR | | |
| | | | | | 2.32) | | |
| DNMT3B | NRG119 | 20 | 30813851 | 30860823 | no | no | NA |

*Survival effect significant using amplitude of gain as a continuous variable
**Trend to worse survival with administration of chemotherapy

TABLE 12
qPCR primers

| Gene Symbol | forward primer sequence | reverse primer sequence |
|---|---|---|
| ABCD2 | GAAAAGAAGCCTCGGACTTTCA | AAGCCAATTTGCATTCCAGGT |
| ABCD2 | CCAAATGGTCCAATGGGTAT | TCAGTCTTTTTCATGTTTTCCG |
| ABCD2 | TCCATGAGCTTTTTGTGCCT | TCAGAGATGTTTTCCCTTCCA |
| ACCN2 | CAGAGGGAAGCAGGAATGAG | TGCTGTTCCCCTATCCAATG |
| ACCN2 | TTCAATCCCAGAACAGGACC | ACAGCCTTACTCTCCAGCTCC |
| ADCY2 | CCTTCCCAACTCACTGTGCT | CCTGGTCATTCGGTGTATCC |
| ADCY2 | CTCCAGTCCAGTTTCCCAAA | CATCCTGGATTGATGACAAAAC |
| AK024870 | TGGTCTTGGACAGTAAGGGAAAATCCA | AGAAATGCCCATTGCTAGCTCAACTT |
| AK024870 | CATCTGTCGTTAAGGAGCAGCAAGAA | TCTCCAAGGAGCTTTCTATGTAAGGGG |
| AK024870 | CCAATTGCCTCGTCATAGCCTGGG | TGCACTGGGTGTGAACTTTAAGAAGCA |
| AK097943 | GCCCGAGATGTTCAAGACAGGGC | ACCAGCAGGAAAACTGGCTGTGTG |
| AK097943 | TGTGCGAAGAGCTGCTGCATGA | AGGGGAAGGCACGGTGTTTGC |
| AK125310 | TGCCAGTGTCCCTTCACCCCT | TGGCAGAGTGATGCCAAGGCTG |
| AK125310 | TGGGAAAGGTGCCGAGACATGA | GCTGGCCAGGTCAGTGCAAC |
| ALG10B | TTGGAAGCAAATTGTTGGTTT | AAGAGATTGTGATTCCACAGAGAA |
| ALG10B | TTCAGCCATATTAACATACATTGACA | CCATTTGTTAACTGGAATCATTCAT |
| ANGPT1 | TGGCAATTTAACATGTGTATTCTTT | CGAATACCTAATTATCCTATTCTGAAA |
| ANGPT1 | CCATTTTTCTATTCTTGGTGGC | GAAGGAGAGGCTTACCTGCT |
| ANKH | TGTCGCTTTTAAGGAAGTGCT | CCAATGCAAAAACTTCCATCT |
| ANKH | GTCATTCCTCTACATGGGCTG | AAACTGACAAACCTATGGGCTG |
| AQP2 | AGACTGTAAGCCCTTTGGGG | GATAGGAGAACGCCATCCAA |
| AQP2 | CCATACTCCCACTTTGTGCC | CGACATTGAAGCACCATTTG |
| ASXH1/ASXL1 | ACCAGCCAAGAGCCGTGTGC | GGGCAAGCTACCCTGCAGCAA |
| ASXH1/ASXL1 | TCAAATGAAGCGCAACAGAGGGGA | AGGGCACGGAGGTTGGTGTTG |
| ATP5G2 | TTTCCTATACCTCCCCAGGC | CTGTCAAACCCTGAGCCAAC |
| ATP5G2 | GGCAGTCTCATGTCCCCTTA | TGTGTCGATGTCCCTTGAAA |
| AZIN1 | AAAGGTAACTTGTGTGTGATTCTGA | GAAGCCAAAGTAAAACATGAGGA |
| AZIN1 | GCAACTTTGAGTCCTTGGCT | AGCTCTCCTGCAGATATGGC |
| B3GNT2 | TCCGGGAATCCTGGGGCCAA | GGGTGGTTGTCCTCTGGGGGT |
| B3GNT2 | GCCGGAGGCTAGCAGAGCCA | GCCGCAGCTCACGCTCCAT |
| B4GALNT | AGAGTCCCTGTGCAAACACC | CCCTTGAACCCCCTTACCTA |
| B4GALNT1 | AATGTGGCAGTCCTCTCAGG | GCTGAGCTATGGGTGAGGAA |
| BAALC | AAATGCAGGGCACATGATCT | GGTTGCTGTCTCCGTGAAAT |
| BAALC | TTTGTGGCTTCTCTTTACAGCTT | CAAACAACATGCAGCAGTGA |
| BAG4 | GGCAGCGGATCCCATGTCGG | GCACATCTCCACCCCCAGGC |
| BAG4 | AGCTTTCGGGGTTCGGCAGC | TGGAGAGCAGCGAAGGGGGT |
| BC035019 | AGAGAAGAGCCTGACGCAGA | AGTGAATGCCGACCTTTGAA |
| BC035019 | TGGCTTGATCTCTCATACAAAGG | TCATTGCATATTTTCAGGGAA |
| BC038465 | TCCCACCCATGCCTTGCTCCA | TGTTCTCCATCTCTTGGAGGCTGAGAC |
| BC038465 | GGAGCAACATGTTTGGCCAAGTTCC | TGGTTTCTCCAACGGCCAGGACT |
| BC038779 | ACCCCACATCTTGGCAACAACGA | TGTGGGACATTGTTCGATGTGATGAA |
| BC038779 | TGAGTGCTTCAGCTTCTGATCCCAT | GGTCTCTTGGGAATCAAATGCCCCT |
| BC042052 | TGGGTCCATTTGAAGCACAGCAGAAG | CATTGGCGGGGCTTGAACCTCA |
| BC042052 | TGCCATGCAACTGAGAAGTGGTTCA | TGCGACAGCATAGCACAGTGGG |
| BCL11B | ACCGTCAGCCGAAGGTCTCGT | GGGACTTTGCTTTGCAGGGCTGA |
| BCL11B | TCTCTTTGGCCCAGAGGTGGGT | TGCCAGTATTGTGAATGCCACGCT |
| BCL11B | GCAGTGGCTGGTGGGCTACG | CTCGGACGACGTGGCGAAGG |
| BRCA2 | GCTCCACCCTATAATTCTGAACC | TTTTACAGGAGATTGGTACAGCG |
| C1orf50 | GCCTCCGTGCACTGAACCCA | GACAGCCAGGTCCTGTGAGAGC |
| C1orf50 | AGCCCAGGGAGTGGGGAGGATA | TGCCTTTCAAGGACCCCCTCGG |
| C20orf112 | TGGCTGCTGGTGGGTAACTGC | CCAGGCTGCCCAGGGAAGAAC |
| C20orf112 | CCCGACGGCAGATGATGACGAC | AGACGCTCAGGGTCCATGCCT |
| CACNB4 | AAAAACGTGGTGTTATTTTTGTGA | ATGCATGCACTCTGCATTTT |
| CACNB4 | ATTCTACAAGGCATGCTGGG | GGGAGAACAAAACATGCAGC |
| CAND1 | AGCCAGGACCCACAGCCCTC | CGCGGCGGATGGTTTCCACT |
| CAND1 | AGCAGCACTGCTAACCATTCCAG | AGCCGCCAGCTCAGGGTTAGA |
| CAND1 | GGAGGCGGGCTTTGGCCTTT | GGCCTCTACGGGGAGCCAGA |
| CCND1 | TTGCGCCTGTGACCACCACC | TGGCCTTTCCCGACCCTGCT |
| CCNK | ACCCAGAAGGGGCAGAAGAACCA | GCCATCCAATGAGGCAACCCCT |
| CCNK | GGACCGGGCCCCTGGGATAAA | TGCAAGGGCACTGATGAGGCT |
| CCT2 | TCCCACGTGCTGTCGATCTTTGG | TGGGCACCGATAAACAGATTCCACA |
| CCT2 | GTGTGGCGTCACTTCCGGCT | TGGTTCCGAGGAGTTCCGCAC |
| CD14 | ACGCCAGAACCTTGTGAGC | GCATGGATCTCCACCTCTACTG |
| CDH18 | TGGAACTGAGGAAGCTGGAC | TTCGATCATGAAAAGGGCAC |
| CDH18 | TGAAAGAACAACTTAGGGGGTC | TCAGGAAGCAAATTCCACAA |
| CDK2 | CTATTGCTTCACCATGGCCT | ATCAGGGATCCTTGGCAACT |
| CDK2 | TCTGACGTCCACCTCCTACC | AGCCCTGAAAAAGTGTCAGC |
| CDK4 | CATTTCTCTACACTAAGGGGTATGTTC | AAGGTAGGGAAAGGGACAAGA |
| CDK4 | GAGGGCAATCTTTGCCTTTA | AGAAAGATGGAGGAGGACCC |
| CDK4 | TGGCCTCGAGATGTATCCCTGCC | TCCATCTCAGGTACCACCGACTGC |
| CDK4 | TTTCCGCGCGCCTCTTTGGC | ACGCAGAGGGCCCGACCATA |
| CENTG1/CR625050 | TCGGGAACCCCCTCCTTCTCCCAT | GCCCAGCCGAGCCTTCAGTCTT |
| CENTG1/CR625050 | CAGGCTGGGCAGATGCTGTGATCT | GGAGACGGCTCACAGCCTGGAAAC |
| COMMD7 | ACCCTCCTCCCAAAAAGCAAGAGC | GTGCAGAAATCCGTGGGGGCT |
| COMMD7 | CCAGGTGATGCTGGGGTGATGC | GCAGGCCAGAGTGTCTCTCGGA |
| COPZ1 | TGACCCAGAACTTCCTCCCCACA | ACCTCAGTGCTGGAGAACTGGCA |
| COPZ1 | GGCCCTCATCCCAACAGCCC | AATCCCTACCCATCCCCGCCC |
| CPSF6 | GGCCCACTTTAAAAGCACCTGACTAGC | AATCAAGTTGACACCCTGCCTCTGC |
| CPSF6 | AGCTCCGCATGTGAACCCAGC | TGTTGGTGGTGGACCTCGGCT |
| CPSF6/AK021534 | CACGTGGGAGTATCCTAAAACTCTGCC | TCGCTAAATGCAGGGTCTGTCCAA |
| CR623026 | TGGGGACCTCAGATTTCCACCCC | TCAAGTCCAGCGCTCTTCCGAGT |
| CR623026 | TCCAGCCTGGGGTTAGGGCA | TGGGAGACCCAAACTGCCGC |
| CR625050 | GTTCACATGGAGGCTGCGGCT | CACCTTTCCTGGGTCACGCCG |
| CR625050 | ACCTGCCCCTCCACTGCACA | AGCGCCTTTCAGGTGCCCTCT |
| CTSC | AGGGCAAGGATCAACTCCAT | TCGTGTAATACATAGGGAATCAAATG |
| CTSC | TCAGTGAGTACAAAATTGCAGATACA | CAAACAGGCAATTATGACACAGA |
| DCD | GCCATGAAGCATCAGCAGCTCAAAAGG | TCTGCTTCCTTGGCTTTGGTGCC |
| DCD | CCAAGGATTTGGTGGCATACCCACT | AGAGCTGTCAGGAAGAGGAGAGTCA |
| DLST | TAGGCCTCGTATCCTGCACT | CCCCAGCTTGTCTTGGATTA |
| DLST | GAGCAAGGTCTTGTTGCCTC | TCGTCGCTGTCCTAACTCCT |
| DNMT3B | AAGGCCACCTCCAAGCGACA | CTCGGAGAACTTGCCATCGCC |
| DNMT3B | TCCGACACCTCTTCGCCCCTC | TGGGTCCTGGCTCTGCCACA |
| DQ515897 | TCCAAGCACTCACTGCCCTCTTG | GCAGGTGAGGCAGGCAGAAACT |
| DQ595898 | GCCTCACTGACTACCTTCAGGGCA | ACCCTTCTGGTTCTCAAGGAGTTTCC |
| DQ595898 | CCAAGCACTCACTGCCCTCTTGC | AGGCAGGTGAGGCAGGCAGAAA |
| DYRK2/AK024870 | TGCCACTGTAAGGTTCTCTCAGCCT | CAGCCAAAGTGACTTCTGTTCGTCCA |
| DYRK2/AK024870 | TCCCTCCATGCTCCAGGTCCA | TCCCACTACCCCCAACACCCA |
| EHBP1/KIAA0903 | CACAGAACCCCAGAAGTCTCAGCAG | CAAAACCTGTGCTTGGGTTGAATCTGT |
| EHBP1/KIAA0903 | GGGAGAAGACTTATGGACCCCAAGCA | TGCAGAGGGTCCAAAGCAAAGGA |
| ERBB3 | CTAACCCCAACAGCCACATC | CCACCACCACTTCCTGAGAT |
| ERBB3 | CTGAGCTTAAAGAGATGAAATAAA | AGGAATTGGGAGGATTTTGC |
| ESPL1 | GCCTCATAACTGTTCTACCTCCA | CATATAAAACACTGGGGAAAATCAC |
| ESPL1 | CAAGCTCCCCGACTCAAGTA | CAGAGAGACAGGCAAGCCAT |
| FAM112B (GTSF1) | ACTGTTGCTTCTTCTTCTACCAGTGGG | TTGTCAGTTTGGAAAGTCACAGGGAGT |
| FAM112B (GTSF1) | GACTCCCTGGACCCTGAGAAGCTAT | AGGAAACCTGCAAGCCCTGATTTGA |
| FGF19 | ACCGGACTGGAGGCCGTGAG | TACCACAGCCCCTGGCAGCA |
| FGF19 | CCTACCCGTGGGGCCCGTAA | CGCAGCGCTCCTGCTCTGAC |
| FGF3 | CCCGCGTCTGGGTTCTCAGC | CCCCCTCCGAGCTCCGACTT |
| FGF3 | AGCGCCGAGTGCGAGTTTGT | CGGGCCCCAGGCGTACTAGA |
| FGF4 | GGGGAACCGAGTGTCGCCCA | AGGGGCTTCCCGAGGCTGAG |
| FGF4 | CTGGTGGCGCTCTCGTTGGC | CCGCTTGATGCCCAGCAGGT |
| FGFR1 | ATGTGTCTGCCCCCTCTATGT | ACAAGAAACGAAGCCAGGGAC |
| FGFR1 | GTAGCGCATTGCGGCGACCT | AGTCCTTGGGTTCCGCGGCT |
| FGFR1 | GTAGCTCCATATTGGACATCCCCAGA | GGGTCCCACTGGAAGGGCATT |
| FIGN | TCTGCGAGTATAGGAAGCTCTC | CCCTGCATGAAGACTGGGT |
| FIGN | TGCAAAGCACTGGCATTTTA | GGGGCTTCTCATTGCATTTAT |
| FIGN | TGAACTGTCACAGAGCAGGC | GCCAGGCTGTTCTGCTTATG |
| FLCN | TGCCAGAGAGTACAGAAGGG | CCGGAGGGACTTGAAGACT |
| FLJ33706 | ACACAGCCAAGCCCTGCTGC | GCCCCAGGAGGCAGACAACG |
| (LOC284805) | | |
| FLJ33706 | CTGTGCAGGGCCGGGATAGC | CCACAGCCCAGCAGGAGCAC |
| (LOC284805) | | |
| FLJ33706 | ATTCTGGGCACCGGCGCTTG | CGGCCGGGGTTCTACCCAGA |
| (LOC284805) | | |
| FRS2 | GCTGAGCTGATCATACACTGACCTGA | GGGGAAAGTGAGCATGAAAAGAACTCC |
| FRS2 | ATGGTGCCTTCCCTCCCTCCA | GTGCATATCTCATCACTCCACAGCAGC |
| FRS2 | ACAGTGATGAACGAAGAGATGCACCC | CCTCTCCTGACCCCTGAGGCAC |
| GAD2 | CTTCCGCATGGTCATCTCAA | CTTGTCCAAGGCGTTCTATTTCTTC |
| GAD2 | ATGTGGCAACCTGTTCTTCC | TTGGGTTTAGAGAGACAACACAGA |
| GAD2 | GTGTGCCAAACTACCGTTCC | ATGTTGGGGGAATGTTGATG |
| GLI1 | TGCCCCCATTGCCCACTTGC | GACCCCCTCCTAGCCTGCCC |
| GLI1 | GCGAGGGGTCCAGGCTCTCT | TGAGGCCTGCTGGGGACAGG |
| GPR158 | CACATTCAAGCAATAACCCACG | CCCATGTCTAGCTCATCCTCAT |
| GPR158 | AAACCTACAGCATCCCACCA | GCTGCAACACGTCACAACAT |
| GPR158 | CTGCATGCAAGTTATGACAGG | ACGACTCTCGGTTGCTAAATG |
| GUCA2A | AATCGCTGAGGACCCGGGCA | GGAGGCAGGCAGTGGGCAAG |
| GUCA2A | TGCACCCATCCCTGGTGAACCT | TGGGCTCCTTGCAGAGAGGCTT |
| HIC1 | GGCGACGACTACAAGAGCAG | CGGAATGCACACGTACAGGTT |
| HNRPA1 | GGCGAAGGTAGGCTGGCAGAT | TGACGGCAGGGTGAAGAGAGACT |
| HNRPA1 | TTCCTTCGGTCGCTGCCACG | TCATGGTTGGCGGAGAGCGG |
| HNRPA1 | TGTTGGCAAAGGAACGTCCTGCT | AGTCGTCCAGTTTCCCACTACCCT |
| HOXC10 | CTATCCGTCCTACCTCTCGCA | ACATGCAGCAGACATTCTCCT |
| HOXC11 | ATGTTTAACTCGGTCAACCTGG | GCATGTAGTAAGTGCAACTGGG |
| HOXC11 | TTCAAATCACGCATCTCTACTCC | TGCACATGTACACACGCACT |
| HOXC11 | AGTAGGGAGATGGGATTGGG | CCCTCCAGGTGGAAAGAAAC |
| HOXC9 | CTCGCTCATCTCTCACGACAA | GACGGAAAATCGCTACAGTCC |
| IFI44 | TCTAAGACCAAAGGGATGTGTTT | AATGTTCTATGCATTTCTTCATCC |
| IFI44 | CACGTAAATTTCCTCACATCACA | TTTGGTCTGTGTTTTCTCCTTTC |
| INHBE | AAAGGAGAAAGAAAATCAACAAATG | GGACCATCACCCTAACCCTT |
| INHBE | AGGGAAGGTCAAGAGGGAGA | AGGGAAGGTCAAGAGGGAGA |
| INHBE | ACCGAGGCGGCTCTTGGACA | CGGCCTGCTCCAGGCTCATT |
| INHBE | TGCACCTGACCAGTCGTCCCA | CTGGAGCCACACTCCCTGGCT |
| IRAK4 | CTGGAAAAAGTCCCACTTCTGAA | AAAAGACTCGCAGGAGCAAAA |
| ITGA7 | CTCTCCCATTCACCCTGTGT | CCCCGACCCTCTAGGTTAAG |
| ITGA7 | AGAACTCCTCCCACCCAACT | CCCACTCTCATCTCACAGCA |
| KIAA0286 | GCAGGGCTAAGGAATTACTGG | CCCTAAGGTATTACCCACAGGC |
| KIAA0286 | TCATGAATGTTTGAAAGGAACAA | TAAGACCCATGGCAAAGAGC |
| KRT1 | AGTTAGACCCAGGGTGTGGA | CAAAACCAAAACAGCACAGAGA |
| KRT1 | TGAAGTTTTCAGATCAGTGGCA | ACAAAGCAGGGTCATAGCCA |
| KRT6A | GGAGGCGGCAGTTCCACCAT | GGACCGAGAGCTAGCAGACGCA |
| KRT6A | CCGAGCCTGATTCCTAGTCCTGCT | TGGATGTGCTGGCCATGGTTCC |
| KRT6B | TGCAGTGTCCCTGAATGGCAAGTG | AGGCAAAGAGAGCAGAGAAAGCAGTG |
| KRT6B | TGCTGCCGCCAGCTCTCAGT | TGGAGGCCAGGGGAGGACAA |
| KRT76 | CTGTCTGAGGAGGGCAGAGCCA | CCCTGGGAACCAGCAGTCTGGA |
| KRT76 | GCAGCTGCCTTACCTTCCAGATGA | ACCCTCCTCTGCCCCAGCAT |
| KRT76 | GCCCCCTCCTATTCCAGCCCA | TGTGGCGGACTCCCCATCCT |
| KRT81 | AAGGGCCAGGACCAGAAC | TCAAGAGCAGAGGAGGAAGG |
| KRT81 | CTGTGTGATCCCCCACTTCT | CTTTCTAGGGTGGCCTTTCC |
| KRT86 (AK057905) | CCTGGTAGTCAATTTGTTGTCCCGAGG | TGGGGAAGAGCTCAGGCAAGAC |
| KRT86 (AK057905) | GTCTGCGGCGGCAGCTGTAA | GTGGCGCGAGGTACTGGCTG |
| KRT86 (AK057905) | TGGCGATCTCTGCGCCTCCA | AGTGCCCACCACCACGTTGC |
| LACRT | GCAGAACCAGCTTCACCCCCAG | AGGTGACCTTGGCTGTCCCCT |
| LACRT | TGCATTGCACCCACACACAACG | GTTGTGTGAGCCAGGACAGAAACCA |
| LACRT | TGGTGGTAATGGGGAGGGGCA | CCTTGCCTCTCTGGGTCATCCTCT |
| LEPRE1/CR623026 | GCCTACATCTGCCACTCAGCCG | ATCCAGGGGGTGCGGTGTCT |
| LEPRE1/CR623026 | TGTGGAAGAGCCGTGGGATTCTCT | GGGTGAACCACAGGGCGATGG |
| LOC284804 | AGTTCCGGGACTGGTGCTTGC | TCGGCGATCCGCTGGTATTTGC |
| LOC284804 | AGCCGGCGAGAAAGGCAAGT | TGGCCCATCTTGGGTTCCCG |
| MAL2 | TTGCCTCCTCCAATGTTCCTC | CAGTTAGCATCAATTTGAGCCAC |
| MAP3K12 | GATGGCTCAGGCTGAAGAAC | CACCAGGATAAAAGCAGGGA |
| MAP3K12 | CGTAGAGCTGTGGCTAAGGG | TATTGCCTTGTTGCTTGCTG |
| MARS | CAGATACAAGCGCTGATGGA | TTGTGCTTTCAGTTCTCGGA |
| MARS | GATTGGCACAGTCAGTCCCT | CAAAGCGCTGCCTTAAACTT |
| MDM1 | AGAGTCCCTTACCATGACCCACAGAT | GGGCTTCTGGTTCTGGTGATGC |
| MDM1 | CGGGCCGAGGCTTTGCTAGG | GAGCCCCCGCTACTCCGACA |
| MDM2 | GCTCATCCTTTACACCAACTCC | CCAAGTACTTCTCATTTAAGACAGAG |
| MDM2 | GAAAAGGAATAAGCCCTGCC | AGACAGGTCAACTAGGGGAAA |
| MDM2 | GTCACATGGCAGCCTGGCCTA | AGCCCAAACTCCCCTCCCTGT |
| MDM2 | TGGAATCTGTTGTTTCCCCCTAAGTTG | GGAACCATGTAACCCAGGCCAAGA |
| MYC | ACGGCCGACCAGCTGGAGAT | TCGTCGTCCGGGTCGCAGAT |
| MYC | TCCGCAACCCTTGCCGCATC | CGCGTCCTTGCTCGGGTGTT |
| MYC.1 | CCTCCACTCGGAAGGACTATC | TCGGTTGTTGCTGATCTGTCT |
| MYC.2 | CCACAGCAAACCTCCTCACAG | GCAGGATAGTCCTTCCGAGTG |
| MYL1 | CCTATGATGCAAGCCATTTCCA | ACACGCAGACCCTCAACAAAG |
| MYL1 | CACAAACAAAGTGTCTGCTGC | GAATGGTGCTTGGATTTGAGA |
| MYL1 | CACCCATGACAAACTCTCCA | CCGTCCAGATTGCTTTGTTT |
| NDRG | GGGATCAGTTTACCTGCCAA | GGCCTGGATTCCTGATCTTT |
| NDRG | GGAACTTGCTTCCCTCTCCT | GCCAATGCTACAAACCCAGT |
| NEUROD4 | TATGCCTTTGGGGAGTATGG | ACAATTTCAGGGAGGCTTGG |
| NEUROD4 | GTGCTTGCAAACCCTTCCTA | CCCTCACTCCAAAACTCAGC |
| NPFF | AACGCTTTGGGAAGAAGTGA | TTGACACTTTTGGGTGTGGA |
| NPFF | CTTCCTGTTTCAGCCCCAG | CTCCAGGATCCCTGGGTATT |
| NUP107 | GCTAAGGAAGTTGCTGCAGAAGCTCAG | TACCCTAATGGGTCAAGTCCCTGGTC |
| NUP107 | GGGCATTTGGATGCCCTAACTGCT | CACCATCCACCCTCCATCAACAAACAA |
| ORAOV1 | CCGCCTCCGGAATGCACAGG | TGGCCACCACAGACTCCCCC |
| ORAOV1 | GCCTCGCCACACATGCCCTT | TGCTGCCGGAGAGGCTGTCA |
| OS9 | TGAGGAGCCTTCACCTCTGT | GTGGGTGCTTCACACCTTTT |
| OS9 | TATTCCCTGCTGCCTACCTG | CTGCTAAGTGTCCTGCCCTC |
| OXCT1 | AAACTTATCATTCCAGTATGCATCTTT | TGCATTTCTTAACATGTATAGCACTCT |
| OXCT1 | ATGGTTAAATGCATACCTTCCC | TGCACATTCTAAGAAGGGTCATT |
| PA2G4 | GAGCTGGAAGCTCAACTGGT | CTTTCATGGGAGGGAGATCA |
| PA2G4 | GATTGCTGGGGGTTTGTAGA | GAGCCCTAGTTTCCTGGGAC |
| PDGFRB | TGATGCCGAGGAACTATTCATCT | TTTCTTCTCGTGCAGTGTCAC |
| PDK1 | GGACAGGAAGTGGACACGAA | TCTTGCTGTCCCTTCCTAGA |
| PPAPDC1B | CCGCTGCTTCCCTGATGGGC | TGGGGAAGCTCTTTCGGCCCT |
| PPAPDC1B | CCGTTCCAGAGACTCATCCAGCCG | ATCGGCTTGGTGGGGAAATACTCCG |
| PPIH | GAGTAAGATAATCTGGACTGGCCCCCG | TCCATGGTCTCTTGATCAAATGGGGCA |
| PPIH | TCCGCGGACCGGGCTTTAGG | TTTGCCACCGCCATGGCTCC |
| PRIM1 | TGGATAAATCCCGAAAAGGA | TCCACAATGGTTTGAGGAGC |
| PRIM1 | TCATCCTAAAACAGGTCGCA | CGGCAGATGAAGCTTATGGT |
| PRMT6 | GCTGTCCACCTCGCCTTTT | TCCTGAAACGTCCGTGTCTTG |
| PSRC1 | CACCGAAGTGACCCAAATGC | GTCTCGGACAGGACTATCCTT |
| PVT1 | AAGAGGATCACCCCAGGAACGCT | ACAGCCCCAAGCTGGGTCTTCA |
| (M34429/M34430) | | |
| PVT1 | TGACACACGCCCGGCACATT | TCCCCCATGGACATCCAAGCTGT |
| (M34429/M34430) | | |
| RAB11F1P1 | AGCTCAACGGGGCAGAGGGA | TGGGAGGGAGGATGGTGCGT |
| RAB11F1P1 | TGCGCAGCTGACCCACGATG | CAGCTCGCGGACCTGGAACTC |
| RAB11FIP1 | TGGGTCTCTTGTGGAGAGCAA | TCCGCATCATGGAATCAATGG |
| RAP1B | ACATCGCCAAACCTCGCCCAG | CGCTACTCTAGGCGCCACGG |
| RAP1B | GGGCTTGAGCCTGACAGCGA | TCCTCCTGCCACTTCCCGCA |
| RHOC.1 | CATCGTCTTCAGCAAGGATCAGTT | TGCCGTCCACCTCAATGTC |
| RHOC.2 | ATGGCTGCAATCCGAAAGAAG | ACAGTAGGGACGTAGACCTCC |
| SCN8A | AGCCATTGTTGCACATTTTG | CCCCATGTACTGGACACAGA |
| SCN8A | GGTAAGAGTTCCATACCGGC | CCCTACCCAGAAGGTGTATGAA |
| SLC11A2 | TTAACAGGGGAAAAGGGAAGA | CACTAGCAGAACCTCAAGGGA |
| SLC11A2 | TGTGTTTATGTGGAATGTTTAAGGA | AGCAGCACAATTATTTCATGTCA |
| SLC35E3 | TGCTGTGGGTTCTCGGTGTTCA | ACACTGGGAAACCATCTATCAGCAAGC |
| SLC35E3 | TGGCATTCTCGCCTATACCCACT | ACCCAATTAAGGACGTTGTGCCAGT |
| SMAD5 | GTCCAGCAGTAAAGCGATTGT | GGGGTAAGCCTTTTCTGTGAG |
| SRD5A1 | GCATTGCTTTGCCTTATCATC | AAGACAACTGAAACAAATGGCA |
| SRD5A1 | TGTTTTGCTGTTGTTGCTTTG | ACAGGTACAGGCTATGAGGGG |
| ST7L | GTGTCCTGAGTGGGTCTGA | CCTTTGTCTCACTTCCCTTATCAAG |
| TBP | CGCAGCGTGACTGTGAGTT | TCCCTCAAACCAACTTGTCAACAG |
| THBS2 | GACACGCTGGATCTCACCTAC | AACTGGTCCTATGAGGTCGCA |
| TMEM17 | CCTTGCTTTCCAAGTTGTTGCAGCATT | AGAGCCGGTCAAAGTCTTGGAGGT |
| TMEM17 | AGCCCGTGTCTGAGGGGGTG | CGGCCCGGCTGAAGTTTCCC |
| TMEM75 | AGACCAACAGCAGATAGTTTCAG | GCACTTACTTTGTGCTATACCCT |
| TNFRSF19 | AGCAGTCAAGATTTGGTTGGTG | CCTGAGTTGATGCTGATTCTACC |
| TP53 | AGCTGGTTAGGTAGAGGGAGTTGTC | GGTTCACCAAGAGGTTGTCAGA |
| TRIP13 | TTCCAATGTTGTGATTCTGACCA | TTCCAATGTTGTGATTCTGACCA |
| TSFM | TGGCCCAGGAGGAATATTTA | GCATTCTCGGTCTGAAGAGG |
| TSFM | TCAGGGGGTGTCGGTAGTAG | GTTTCTGCTGCCTCTTCACC |
| TSPAN31 | GGGCCTGGGTCTGGTGTCCA | ACAGCACCCACCAGTCCAGC |
| TSPAN31 | CGGTCCCCAATACCCTCCCCC | AGCAGGCAAAGCCGCCACAA |
| TUBA2 | GCTGGGAACTGTACTGCCTG | GTCCCCACCACCAATGGTTT |
| UBE1L2 | AGTTCCTTCCCAGTCACAACC | AATTACCAAAGGGTACGTGGC |
| UBE1L2 | AGTTGAGCTCTTGAGGATCAGA | TGACATAGAGCAAAATGAACACA |
| UTP18 | TTGAGTCACAAGAGAAGCCTGT | AGCTGGATCTATAAATCATTTTCCA |
| UTP18 | AAGTGGATACTTTGCCTTGGG | CTATACATCAGGGCCTTGCC |
| WHSC1L1 | GGAAAAACCTTCCCCTCCACAGCC | TTCAGGTGAGCCAGTCTTCTTTGGA |
| WHSC1L1 | CCACCTCAACTCATTGACTCCGCC | GGTGTCTGGCCACCATCTTCAGC |
| WISP1 | CCAGTTGGTGACTGGGAAAG | AAACAGGGGGAAAATATGGG |
| WISP1 | TTCGTTCTGCTGACCAAATG | AAACAACCGGTAAACCTCCA |
| YAF2 | GTTACTGGGACTGTAGCGTCT | TTCCGCACATCGCACATCAT |
| YAF2 | TTAAAGGCTTTCTCATGAGGCT | AAGAGCGAATCCATTCCAGA |
| YAF2 | AAATGGTCTAGAAGTTTTCGTTTCC | AAAAAGCGAGTGGCGGA |
| YBX1 | TACCGACGCAGACGCCCAGA | AGCCTCGGGAGCGGACGAAT |
| YEATS4 | GCCAGCCCCGGTCTCTTTCC | CGCCGGAGTCAGGCCCAAAT |
| YEATS4 | TCACCGCCGTGAGCCCAAGT | TCGCCGCTCCCCTCAGAGAC |
| ZBTB39 | TAAAACCCTTCCCCTGTCCA | TTAGCTATTCAAGGTGGGGG |
| ZBTB39 | CCCCAAATAGTAGATGTCTAAAATCA | ACAATGGAATATAAAAGAATCAGATGT |

TABLE 13
BAC clones that lie within high-amplitude MCRs

| | | MCR ID |
|---|---|---|
| | | that |
| | | clone |
| BAC | | lies |
| clone ID 1 | BAC clone ID 2 | within |
| N0316O06 | RP11-316O6 | NRG4 |
| N0164K22 | RP11-164K22 | NRG4 |
| N0399E06 | RP11-399E6 | NRG4 |
| N1006C08 | RP11-1006C8 | NRG4 |
| N0595K03 | RP11-595K3 | NRG4 |
| N0462E20 | RP11-462E20 | NRG4 |
| N0092H18 | RP11-92H18 | NRG4 |
| N0413J19 | RP11-413J19 | NRG4 |
| N0499B14 | RP11-499B14 | NRG4 |
| N0045C15 | RP11-45C15 | NRG4 |
| N0799L22 | RP11-799L22 | NRG4 |
| N0558M13 | RP11-558M13 | NRG4 |
| N0096H10 | RP11-96H10 | NRG4 |
| N0105J15 | RP11-105J15 | NRG4 |
| N0483I17 | RP11-483I17 | NRG4 |
| N0336K05 | RP11-336K5 | NRG4 |
| N0772D22 | RP11-772D22 | NRG11 |
| N0270B14 | RP11-270B14 | NRG11 |
| N0093M19 | RP11-93M19 | NRG11 |
| N0342G13 | RP11-342G13 | NRG11 |
| N0598I11 | RP11-598I11 | NRG11 |
| N0017L22 | RP11-17L22 | NRG11 |
| N0312H10 | RP11-312H10 | NRG11 |
| M2010B19 | CTD-2010B19 | NRG11 |
| N0257N14 | RP11-257N14 | NRG11 |
| N0678E17 | RP11-678E17 | NRG56 |
| N0380B11 | RP11-380B11 | NRG56 |
| N0745K06 | RP11-745K6 | NRG56 |
| N0371M15 | RP11-371M15 | NRG56 |
| N0095I18 | RP11-95I18 | NRG56 |
| N0621B01 | RP11-621B1 | NRG56 |
| F0631H19 | RP13-631H19 | NRG56 |
| N0332C08 | RP11-332C8 | NRG56 |
| N0319J12 | RP11-319J12 | NRG56 |
| F0509O17 | RP13-509O17 | NRG56 |
| F0620O23 | RP13-620O23 | NRG56 |
| M2015B18 | CTD-2015B18 | NRG56 |
| F0580P15 | RP13-580P15 | NRG56 |
| N0275E14 | RP11-275E14 | NRG56 |
| M2225N15 | CTD-2225N15 | NRG56 |
| N0264P13 | RP11-264P13 | NRG56 |
| N0156L03 | RP11-156L3 | NRG56 |
| N0594D10 | RP11-594D10 | NRG56 |
| N0389E22 | RP11-389E22 | NRG56 |
| N0601G22 | RP11-601G22 | NRG56 |
| N0636F12 | RP11-636F12 | NRG56 |
| M2385A20 | CTD-2385A20 | NRG56 |
| N0350N15 | RP11-350N15 | NRG56 |
| N0148D21 | RP11-148D21 | NRG56 |
| N0675F06 | RP11-675F6 | NRG56 |
| N0734M08 | RP11-734M8 | NRG56 |
| N0495O10 | RP11-495O10 | NRG56 |
| N0794F05 | RP11-794F5 | NRG56 |
| N0690P09 | RP11-690P9 | NRG56 |
| N0288B17 | RP11-288B17 | NRG58 |
| N0336P08 | RP11-336P8 | NRG58 |
| N0367L07 | RP11-367L7 | NRG58 |
| N0472A17 | RP11-472A17 | NRG58 |
| N0440N18 | RP11-440N18 | NRG58 |
| N0237F24 | RP11-237F24 | NRG58 |
| F0597L24 | RP13-597L24 | NRG74 |
| N0409P16 | RP11-409P16 | NRG74 |
| N0211G23 | RP11-211G23 | NRG74 |
| N0683C06 | RP11-683C6 | NRG74 |
| N0657B01 | RP11-657B1 | NRG74 |
| M2009H02 | CTD-2009H2 | NRG74 |
| N0699M19 | RP11-699M19 | NRG74 |
| M2192B11 | CTD-2192B11 | NRG74 |
| N0124K14 | RP11-124K14 | NRG74 |
| N0681H17 | RP11-681H17 | NRG74 |
| M2234J21 | CTD-2234J21 | NRG74 |
| N0775I17 | RP11-775I17 | NRG74 |
| N0278A17 | RP11-278A17 | NRG74 |
| N0804L21 | RP11-804L21 | NRG74 |
| N0599F23 | RP11-599F23 | NRG74 |
| N0626H12 | RP11-626H12 | NRG74 |
| N0345C10 | RP11-345C10 | NRG74 |
| N0517E18 | RP11-517E18 | NRG74 |
| N0347I13 | RP11-347I13 | NRG74 |
| M2011L13 | CTD-2011L13 | NRG74 |
| N0574F24 | RP11-574F24 | NRG74 |
| N0440D23 | RP11-440D23 | NRG74 |
| F0495C07 | RP13-495C7 | NRG79 |
| N0195M24 | RP11-195M24 | NRG79 |
| N0845M18 | RP11-845M18 | NRG79 |
| N0699F03 | RP11-699F3 | NRG79 |
| N0797O20 | RP11-797O20 | NRG79 |
| N0096P03 | RP11-96P3 | NRG79 |
| M2013M19 | CTD-2013M19 | NRG79 |
| N0593B08 | RP11-593B8 | NRG79 |
| N0417B20 | RP11-417B20 | NRG79 |
| N0641A06 | RP11-641A6 | NRG79 |
| N0707F10 | RP11-707F10 | NRG80 |
| N0185A01 | RP11-185A1 | NRG80 |
| N0615N13 | RP11-615N13 | NRG80 |
| N0722G21 | RP11-722G21 | NRG80 |
| N0442B16 | RP11-442B16 | NRG80 |
| N0383J07 | RP11-383J7 | NRG80 |
| N0192J19 | RP11-192J19 | NRG80 |
| M2265L24 | CTD-2265L24 | NRG80 |
| N0681J20 | RP11-681J20 | NRG80 |
| N0653N18 | RP11-653N18 | NRG80 |
| N0213J12 | RP11-213J12 | NRG81 |
| N0746D11 | RP11-746D11 | NRG81 |
| N0799H16 | RP11-799H16 | NRG81 |
| N0571M06 | RP11-571M6 | NRG81 |
| N0066N19 | RP11-66N19 | NRG81 |
| N0672O16 | RP11-672O16 | NRG81 |
| N0369G07 | RP11-369G7 | NRG81 |
| N0277A02 | RP11-277A2 | NRG81 |
| N0620J15 | RP11-620J15 | NRG81 |
| N0549D07 | RP11-549D7 | NRG81 |
| N0489P06 | RP11-489P6 | NRG81 |
| N0016E13 | RP11-16E13 | NRG81 |
| N0694B03 | RP11-694B3 | NRG81 |
| N0055F19 | RP11-55F19 | NRG81 |
| N0491C17 | RP11-491C17 | NRG81 |
| N0071C21 | RP11-71C21 | NRG81 |
| N0267H12 | RP11-267H12 | NRG81 |
| N0136P02 | RP11-136P2 | NRG81 |
| N0742J10 | RP11-742J10 | NRG81 |
| N0782O11 | RP11-782O11 | NRG81 |
| N0182F04 | RP11-182F4 | NRG82 |
| N0118B13 | RP11-118B13 | NRG82 |
| M2214L24 | CTD-2214L24 | NRG82 |
| F0530J15 | RP13-530J15 | NRG82 |
| N0587G17 | RP11-587G17 | NRG82 |
| N0745O10 | RP11-745O10 | NRG82 |
| N0293H23 | RP11-293H23 | NRG82 |
| N0242M13 | RP11-242M13 | NRG82 |
| N0263A04 | RP11-263A4 | NRG82 |
| N0559K12 | RP11-559K12 | NRG82 |
| N0640G12 | RP11-640G12 | NRG82 |
| N0607F06 | RP11-607F6 | NRG82 |
| N0597A07 | RP11-597A7 | NRG82 |
| N0654O12 | RP11-654O12 | NRG82 |
| N0328H16 | RP11-328H16 | NRG82 |
| N0528M24 | RP11-528M24 | NRG82 |
| N0612H02 | RP11-612H2 | NRG82 |
| N0350A05 | RP11-350A5 | NRG82 |
| N0365P01 | RP11-365P1 | NRG82 |
| N0667H20 | RP11-667H20 | NRG82 |
| N0207E06 | RP11-207E6 | NRG82 |
| N0043N05 | RP11-43N5 | NRG82 |
| N0404H13 | RP11-404H13 | NRG82 |
| N0554D04 | RP11-554D4 | NRG82 |
| M2305I15 | CTD-2305I15 | NRG82 |
| N0044D17 | RP11-44D17 | NRG82 |
| N0679J04 | RP11-679J4 | NRG82 |
| N0104O18 | RP11-104O18 | NRG82 |
| N0081H14 | RP11-81H14 | NRG82 |
| N0185H13 | RP11-185H13 | NRG82 |
| N0392J17 | RP11-392J17 | NRG82 |
| D2538A02 | CTD-2538A2 | NRG82 |
| N0450G15 | RP11-450G15 | NRG82 |
| N0797C20 | RP11-797C20 | NRG82 |
| N0611O02 | RP11-611O2 | NRG82 |
| F0618A08 | RP13-618A8 | NRG82 |
| M2067J14 | CTD-2067J14 | NRG82 |
| N0675P21 | RP11-675P21 | NRG82 |
| N0204P07 | RP11-204P7 | NRG82 |
| N0584J05 | RP11-584J5 | NRG82 |
| N0324P09 | RP11-324P9 | NRG82 |
| N0072P21 | RP11-72P21 | NRG82 |
| N0426B12 | RP11-426B12 | NRG82 |
| N0663D20 | RP11-663D20 | NRG82 |
| N0159A18 | RP11-159A18 | NRG82 |
| N0267B05 | RP11-267B5 | NRG82 |
| N0023C15 | RP11-23C15 | NRG82 |
| N0382J04 | RP11-382J4 | NRG82 |
| N0607D03 | RP11-607D3 | NRG82 |
| N0015L03 | RP11-15L3 | NRG82 |
| N0354A24 | RP11-354A24 | NRG89 |
| N0109L09 | RP11-109L9 | NRG89 |
| N0095F16 | RP11-95F16 | NRG89 |
| N0177F03 | RP11-177F3 | NRG89 |
| N0063E01 | RP11-63E1 | NRG89 |
| N0061O01 | RP11-61O1 | NRG89 |
| N0815L01 | RP11-815L1 | NRG89 |
| N0068I08 | RP11-68I8 | NRG89 |
| N0075N22 | RP11-75N22 | NRG89 |
| N0148G13 | RP11-148G13 | NRG89 |
| N0468P09 | RP11-468P9 | NRG89 |
| N0057E12 | RP11-57E12 | NRG89 |
| N0415J21 | RP11-415J21 | NRG89 |
| N0430I09 | RP11-430I9 | NRG89 |
| N0634B02 | RP11-634B2 | NRG89 |
| N0594K17 | RP11-594K17 | NRG89 |
| N0793L22 | RP11-793L22 | NRG89 |
| N0724J12 | RP11-724J12 | NRG119 |
| N0610D23 | RP11-610D23 | NRG119 |
| N0815L24 | RP11-815L24 | NRG119 |

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

1. Azzoli C G, Park B J, Pao W. Molecularly tailored adjuvant chemotherapy for resected non-small cell lung cancer: a time for excitement and equipoise. J Thorac Oncol (2008); 3: 84-93.
2. Balsara B R, Testa J R. Chromosomal imbalances in human lung cancer. Oncogene (2002); 21: 6877-83.
3. Blayeri E, Brewer J L, Roydasgupta R, et al. Bladder cancer stage and outcome by array-based comparative genomic hybridization. Clin. Cancer Res. 2005; 11(19 Pt 1):7012-22.
4. Cappuzzo F, Hirsch F R, Rossi E, et al. Epidermal growth factor receptor gene and protein and gefitinib sensitivity in non-small-cell lung cancer. J. Natl. Cancer Inst. 2005; 97(9):643-55.
5. Climent J, Garcia J L, Mao J H, Arsuaga J, Perez-Losada J. Characterization of breast cancer by array comparative genomic hybridization. Biochem. Cell Biol. (2007a); 85(4):497-508.
6. Climent J et al. Deletion of chromosome 11q predicts response to anthracycline-based chemotherapy in early breast cancer. Cancer Res (2007b); 67 (2): 818-26.
7. Canadian Cancer Society/National Cancer Institute of Canada: Canadian Cancer Statistics 2008, Toronto, Canada, 2008.
8. Chen W et al. Array comparative genomic hybridization reveals genomic copy number changes associated with outcome in diffuse large B-cell lymphomas. Blood (2006); 107: 2477-85.
9. Chi B, DeLeeuw R J, Coe B P, MacAulay C, Lam W L (2004). SeeGH—a software tool for visualization of whole genome array comparative genomic hybridization data. BMC Bioinformat 5(1):13.
10. Chi et al. “MD-SeeGH: a platform for integrative analysis of multi-dimensional genomic data”, BMC Bioinformatics 2008 May 20; 9:243. [
11. Choi et al. Comparative genomic hybridization array analysis and real time PCR reveals genomic alterations in squamous cell carcinomas of the lung. Lung Cancer (2007); 55: 43-51.
12. Coe B P, Lockwood W W, Girard L, et al. Differential disruption of cell cycle pathways in small cell and non-small cell lung cancer. Br. J. Cancer. 2006; 94(12):1927-35.
13. Dehan E, Ben-Dor A, Liao W, et al. Chromosomal aberrations and gene expression profiles in non-small cell lung cancer. Lung Cancer. 2007; 56(2):175-84.
14. Dhesy-Thind B et al. HER2/neu in systemic therapy for women with breast cancer: a systematic review. Breast Cancer Res Treat (2007); DOI 10.1007/s10549-007-9656-y (published online).
15. Diskin et al. (2006) STAC: A method for testing the significance of DNA copy number aberrations across multiple array-CGH experiments. Genome Research 16: 1149-58.
16. Fensterer H, Radlwimmer B, Sträter J, et al. Matrix-comparative genomic hybridization from multicenter formalin-fixed paraffin-embedded colorectal cancer tissue blocks. BMC Cancer. 2007; 7:58.
17. Han W, Han M, Kang J J, et al. Genomic alterations identified by array comparative genomic hybridization as prognostic markers in tamoxifen-treated estrogen receptor-positive breast cancer. BMC Cancer. 2006; 6:92.
18. Hirsch F R, Varella-Garcia M, Cappuzzo F, et al. Combination of EGFR gene copy number and protein expression predicts outcome for advanced non-small-cell lung cancer patients treated with gefitinib. Ann. Oncol. 2007; 18(4):752-60.
19. Höglund M, Gisselsson D, Hansen G B, Mitelman F. Statistical dissection of cytogenetic patterns in lung cancer reveals multiple modes of karyotypic evolution independent of histological classification. Cancer Genet. Cytogenet. 2004; 154(2):99-109.
20. Idbaih A, Marie Y, Lucchesi C, et al. BAC array CGH distinguishes mutually exclusive alterations that define clinicogenetic subtypes of gliomas. Int. J. Cancer. 2008; 122(8):1778-86.
21. Jaffe E S. Pathology and Genetics: Tumours of Haematopoietic and Lymphoid Tissues. Intl Agency for Research on Cancer; 2003.
22. Jiang et al. Genomic profiles in stage I primary non small cell lung cancer using comparative genomic hybridization analysis of cDNA microarrays. Neoplasia (2004); 6(5): 623-35.
23. Jong et al. (2004) Breakpoint identification and smoothing of array comparative genomic hybridization data. Bioinformatics 20(18): 3636-7.
24. Khojasteh M et al. A stepwise framework for the normalization of array CGH data. BMC Bioinformatics. 2005; 18(6):274.
25. Kim M, Yim S, Kwon M, et al. Recurrent genomic alterations with impact on survival in colorectal cancer identified by genome-wide array comparative genomic hybridization. Gastroenterology. 2006; 131(6):1913-24.
26. Massion P P et al. Genomic copy number analysis of non-small cell lung cancer using array comparative genomic hybridization: implications of the phosphatidylinositol 3-kinase pathway. Cancer Res (2002); 62: 3636-40.
27. Massion P P, Taflan P M, Jamshedur Rahman S M, et al. Significance of p63 amplification and overexpression in lung cancer development and prognosis. Cancer Res. 2003; 63(21):7113-21.
28. Mayr D, Kanitz V, Anderegg B, et al. Analysis of gene amplification and prognostic markers in ovarian cancer using comparative genomic hybridization for microarrays and immunohistochemical analysis for tissue microarrays. Am. J. Clin. Pathol. 2006; 126(1):101-9.
29. Patel A, Kang S, Lennon P A, et al. Validation of a targeted DNA microarray for the clinical evaluation of recurrent abnormalities in chronic lymphocytic leukemia. Am. J. Hematol. 2008; 83(7):540-6.
30. Rubio-Moscardo F et al. Mantle-cell lymphoma genotypes identified with CGH to BAC microarrays define a leukemic subgroup of disease and predict patient outcome. Neoplasia (2005); 105(11): 4445-54.
31. Shah et al. (2006) Integrating copy number polymorphisms into array CGH analysis using a robust HMM. Bioinformatics 22(14): e431-9.
32. Shibata et al. Genetic classification of lung adenocarcinoma based on array-based comparative genomic hybridization analysis: its association with clinicopathologic features. Clin Cancer Res (2005); 11(17): 6177-85.
33. Slamon D J, Clark G M, Wong S G, Levin W J, Ulrich A, McGuire W L. Human breast cancer: correlation of relapse and survival with amplification of HER-2/neu oncogene. Science (1987); 235: 177-82.
34. Takano T, Ohe Y, Sakamoto H, et al. Epidermal growth factor receptor gene mutations and increased copy numbers predict gefitinib sensitivity in patients with recurrent non-small-cell lung cancer. J. Clin. Oncol. 2005; 23(28):6829-37.
35. Tagawa H, Suguro M, Tsuzuki S, et al. Comparison of genome profiles for identification of distinct subgroups of diffuse large B-cell lymphoma. Blood. 2005:106(5):1770-7.
36. Thomas R K, Weir B, Meyerson M. Genomic approaches to lung cancer. Clin Cancer Res (2006); 12(14 Suppl): 4384s-91s.
37. Tomioka N, Oba S, Ohira M, et al. Novel risk stratification of patients with neuroblastoma by genomic signature, which is independent of molecular signature. Oncogene. 2008; 27(4):441-9.
38. Tonon G et al. High-resolution genomic profiles of human lung cancer. PNAS (2005); 102(27): 9625-30.
39. Weir B A et al. Characterizing the cancer genome in lung adenocarcinoma. Nature (2007); 450(7171):893-8.
40. Weiss M M et al. Genomic alterations in primary gastric adenocarcinomas correlate with clinicopathological characteristics and survival. Cellular Oncology (2004); 26: 307-17.
41. Venkatraman E S, Olshen A B (2007). A faster circular binary segmentation algorithm for the analysis of array CGH data. Bioinformatics 23(6): 657-63.
42. Watson S K, deLeeuw R L, Horsman D E, Squire J A, Lam W L (2007). Cytogenetically balanced translocations are associated with focal copy number alterations. Hum Genet. 120: 795-805.
43. Winton et al. (2005) Vinorelbine plus cisplatin vs. observation in resected non-small-cell lung cancer. New Eng J Med 352: 2589-97.
44. Zhao et al. Homozygous deletions and chromosome amplifications in human lung carcinomas revealed by single nucleotide polymorphism array analysis. Cancer Res (2005); 65: 5561-70.

1. A method for determining a lung cancer prognosis predicting tumour responsiveness and/or likelihood of improved survival with chemotherapy in a subject, the method comprising: (a) determining a genomic profile comprising detecting one or more genomic alterations in one or more of chromosomes 2, 11, 4, 5, 7, 9, 12, 17, 19, 20, 8, 1, 13, 16, 6 and/or 14 listed in Tables 1 to 11, in a biological sample from the subject; wherein the prognosis is determined to be poor when the genomic profile comprises a gain of all or part of one or more minimal common regions (MCRs) and/or genes within chromosomes 1, 2, 11, 4, 5, 6, 7, 9, 12, 14, 16, 17, 19 and 20, listed as associated with poor prognosis in Tables 1, 2, 5, 9, 10, and 11, and/or a loss of all or part of one or more MCRs and/or genes within chromosomes 1, 5, 8, 13 and 16 listed as associated with poor prognosis in Tables 3 and 7; and the prognosis is determined to be good when the genomic profile comprises a genomic gain of all or part of a MCR and/or gene within chromosome 8 listed as associated with good prognosis in Table 6; and/or a loss of one or more MCRs and/or genes within chromosome 6 or 14 listed as associated with good prognosis in Table 8, relative to a control.

2. (canceled)

3. The method of claim 2, wherein the gain comprises a gain in all or part of one or more of Table 11 genes FGF3, FAM112B, TSFM, NUP107 and/or MDM2; or wherein the MCR listed as associated with poor prognosis is selected from a MCR listed in Table 10.

4. The method of claim 1 comprising after step (a) the step: (b) comparing the genomic profile with one or more controls.

5. (canceled)

6. The method of claim 1, wherein the prognosis is determined to be poor when the genomic profile comprises a gain of all or part of a gene listed in Table 5, 9, and/or 11 associated with poor prognosis and/or comprises a loss of all or part of a gene listed in Table 7, and the prognosis is determined to be good when the genomic profile comprises a gain of all or part of gene listed in Table 6 or a loss of all or part of a gene listed in Table 8 relative to the control.

7. The method of claim 1, wherein the method of determining a genomic profile comprises: determining a hybridization pattern using one or more chromosomal probes in the biological sample from the subject, wherein the one or more probes hybridze specifically to one or more MCRs and/or genes listed in Tables 1 to 11.

8. (canceled)

9. The method of claim 6, wherein the gain associated with good prognosis comprises all or part of RAB11FIP1 and/or the loss associated with good prognosis comprises all or part of a gene listed in Table 8.

10. The method of claim 4, wherein the one or more controls comprise a control copy number such as centromere copy number or a control gene on the same or different chromosome.

11. (canceled)

12. (canceled)

13. (canceled)

14. The method of claim 1, wherein the lung cancer is non-small cell lung cancer (NSCLC), early stage NSCLC, squamous cell carcinoma or adenocarcinoma.

15. The method of claim 1, comprising detecting the expression level of a gene listed in Table 5, 6, 7, 8, 9 and/or 11, wherein the expression level of the gene all or partly gained or lost is increased or decreased respectively, relative to a control expression level.

16. (canceled)

17. The method of claim 1 for selecting a treatment regimen for a subject with lung cancer, the method comprising: (a) determining a genomic profile comprising detecting a genomic alteration in one or more genes selected from Table 5, 9 and/or 11 and/or 7 in a biological sample from the subject; (b) selecting a treatment for the subject by comparing the genomic profile with one or more controls, wherein the treatment selected comprises chemotherapy when the genomic profile comprises a gain of all or part of one or more genes associated with improved survival with chemotherapy including the following genes: MFSD7, D4S234E, ACOX3, SRD5A1, AQP2, ACCN2, SLC11A2, SCN8A, KRT81, KRT1, ESPL1, NPFF, ATP5G2, HOXC11, NEUROD4, ZBTB39, KIAA0286, INHBE, MARS, B4GALNT1, TSFM, DNMT3B, BAALC, ANGPT1, MYC, WISP1, KRT81, KRT1, NEUROD4, PA2G4, GUCA2A, PPIH, LEPRE1, CR623026, C1orf50, DQ515898, DQ515897, MYC FGF3, KRT81, KRT1, FAM112B, B4GALNT1, CENTG1, and/or BCL11B; and/or a loss of all or part of one or more genes associated with improved survival with chemotherapy including the following genes: RHOC, ATP2C2, ZDHHC7, COC4I1, FOXF1 relative to the control and/or wherein the treatment comprises a non-chemotherapy treatment and/or a non-platinum analog, a vinca alkyloid or a combination thereof chemotherapy treatment, when the genomic profile comprises a gain of all or part of one or more of AK024870 and CPSF6.

18. The method of claim 1, wherein the biological sample is selected from the group consisting of lung tissue, lung cells, lung biopsy and sputum, including formalin fixed, paraffin embedded and fresh frozen specimens.

19. (canceled)

20. (canceled)

21. (canceled)

22. (canceled)

23. (canceled)

24. (canceled)

25. (canceled)

26. The method of claim 1, wherein the genomic alteration, MCR and/or gene gain or loss is determined by array CGH, FISH, chromagen in situ hybridization (CISH) or PCR.

27. The method of claim 1 for determining a likelihood of improved survival in a lung cancer subject who was or is receiving a chemotherapeutic treatment, comprising determining the presence or absence of a gain or loss of all or part of a MCR and/or gene associated with improvement with chemotherapy, predicting the likelihood of improved survival and/or predisposition to platinum analogs, vinca alkaloids and/or a combination thereof according to the presence or absence of the MCR or gene gain or loss compared to a control, wherein detecting a MCR and/or gene associated with improvement with chemotherapy predicts likelihood of improved survival compared to a control having the same gain or loss who has not received and/or is not receiving chemotherapy, and/or is indicative of a favourable predisposition of the subject to respond to platinum analogs, vinca alkaloids and/or a combination thereof.

28. (canceled)

29. The method of claim 1, for treating a subject with lung cancer comprising determining the presence or absence of a gain or loss of a MCR or gene associated with improvement with chemotherapy in a subject with lung cancer and administering chemotherapy to a subject with at least one gain or loss associated with improvement with chemotherapy.

30. The method of claim 29 wherein the chemotherapy is a platinum analog, a vinca alkaloid or a combination thereof.

31. The method of claim 30 wherein the platinum analog is selected from cisplatin, paraplatin, carboplatin, oxaliplatin and satraplatin in either IV or oral form and/or wherein the vinca alkyloid is selected from vinorelbine, vincristine, vinblastine, vindesine and vinflunine in either IV or oral form.

32. (canceled)

33. A composition comprising two or more detection agents for detecting the presence or absence of a MCR and/or gene gain or loss associated with prognosis, wherein each detection agent comprises a hybridization probe; or a primer and/or a primer pair for amplifying one or more genomic alterations listed in Tables 1 to 11 for use in the method of claim 1.

34. (canceled)

35. The composition of claim 33 wherein the probe comprises at least 8, 10, 15, 20, 25, 50, 75, 100, 150, 200, 250, 400, or 500 contiguous nucleotides hybridizable and/or complementary to a gene listed in Table 5, 6, 7, 8, 9 and/or 11, or a genomic region alteration such as a MCR and/or region flanking a MCR described herein, for example in Tables 1, 2, 3, 4 and/or 10 and/or comprises at least 90, 95, 96, 97, 98, 99, 99.5, 99.9% identityl to at least 8, 10, 15, 20, 25, 50, 75, 100, 150, 200, 250, 400, or 500 contiguous nucleotides of a gene listed in Table 5, 6, 7, 8, 9 and/or 11, and/or a MCR and/or region flanking a MCR described herein, for example in Table 1, 2, 3, 4 and/or 10.

36. (canceled)

37. (canceled)

38. (canceled)

39. A kit for determining lung cancer prognosis and/or tumour responsiveness according to claim 1 in a subject, the kit comprising two or more detection agents probe, wherein the two or more detection agents are each a probe to a MCR and/or gene listed in Tables 1 to 11.

40. The kit of claim 39, wherein each detection agent comprises one or more gene expression probes, or a set of probes specific for a gene expression product of a gene listed in Tables 5, 6, 7, 8, 9 and/or 11, or an array with one or more probes for one or more MCRs or genes gained or lost described herein and labeling reagents for labeling the subject sample DNA comprises a primer set for amplifying all or part of a MCR or gene listed in any one of Tables 1 to 11 associated with prognosis, optionally comprising one or more of the primers listed in Table 12.

41. (canceled)

42. (canceled)

43. (canceled)

44. (canceled)

45. (canceled)

46. The method of claim 1 wherein the method comprises (a) determining a hybridization pattern of a chromosomal probe or a set of chromosomal probes in a biological sample from the subject, wherein the probe or probeset is targeted to all or part of one or more MCRs listed in the provided tables, including but not limited to NRG4 on the short arm of chromosome 1 (1p), NRG58 on 8q, NRG74 on 11q, NRG79 on 12q, NRG80 on 12q, NRG81 on 12q, NRG82 on 12q, and/or NRG89 on 14q; (b) determining the prognosis and/or predicting the response to chemotherapy for a patient with lung cancer based on the hybridization pattern, wherein the prognosis is determined to be poor without chemotherapy when the hybridization pattern indicates a gain of DNA copy number at an MCR on 11q and/or a gain at an MCR on 12q and/or a gain at an MCR on 14q relative to a control; and/or the prognosis is determined to be good when treated with chemotherapy when the hybridization pattern indicates a gain of DNA copy number within an MCR on 1p and/or 8q and/or 11q and/or 12q and/or 14q.


SEQUENCE LISTING (534 sequences)

SEQ ID NO: 1 - DNA, 22 bp, Homo sapiens
gaaaagaagcctcggactttca

SEQ ID NO: 2 - DNA, 21 bp, Homo sapiens
aagccaatttgcattccaggt

SEQ ID NO: 3 - DNA, 20 bp, Homo sapiens
ccaaatggtccaatgggtat

SEQ ID NO: 4 - DNA, 22 bp, Homo sapiens
tcagtctttttcatgttttccg

SEQ ID NO: 5 - DNA, 20 bp, Homo sapiens
tccatgagctttttgtgcct

SEQ ID NO: 6 - DNA, 21 bp, Homo sapiens
tcagagatgttttcccttcca

SEQ ID NO: 7 - DNA, 20 bp, Homo sapiens
cagagggaagcaggaatgag

SEQ ID NO: 8 - DNA, 20 bp, Homo sapiens
tgctgttcccctatccaatg

SEQ ID NO: 9 - DNA, 20 bp, Homo sapiens
ttcaatcccagaacaggacc

SEQ ID NO: 10 - DNA, 21 bp, Homo sapiens
acagccttactctccagctcc

SEQ ID NO: 11 - DNA, 20 bp, Homo sapiens
ccttcccaactcactgtgct

SEQ ID NO: 12 - DNA, 20 bp, Homo sapiens
cctggtcattcggtgtatcc

SEQ ID NO: 13 - DNA, 20 bp, Homo sapiens
ctccagtccagtttcccaaa

SEQ ID NO: 14 - DNA, 22 bp, Homo sapiens
catcctggattgatgacaaaac

SEQ ID NO: 15 - DNA, 27 bp, Homo sapiens
tggtcttggacagtaagggaaaatcca

SEQ ID NO: 16 - DNA, 26 bp, Homo sapiens
agaaatgcccattgctagctcaactt

SEQ ID NO: 17 - DNA, 26 bp, Homo sapiens
catctgtcgttaaggagcagcaagaa

SEQ ID NO: 18 - DNA, 27 bp, Homo sapiens
tctccaaggagctttctatgtaagggg

SEQ ID NO: 19 - DNA, 24 bp, Homo sapiens
ccaattgcctcgtcatagcctggg

SEQ ID NO: 20 - DNA, 27 bp, Homo sapiens
tgcactgggtgtgaactttaagaagca

SEQ ID NO: 21 - DNA, 23 bp, Homo sapiens
gcccgagatgttcaagacagggc

SEQ ID NO: 22 - DNA, 24 bp, Homo sapiens
accagcaggaaaactggctgtgtg

SEQ ID NO: 23 - DNA, 22 bp, Homo sapiens
tgtgcgaagagctgctgcatga

SEQ ID NO: 24 - DNA, 21 bp, Homo sapiens
aggggaaggcacggtgtttgc

SEQ ID NO: 25 - DNA, 21 bp, Homo sapiens
tgccagtgtcccttcacccct

SEQ ID NO: 26 - DNA, 22 bp, Homo sapiens
tggcagagtgatgccaaggctg

SEQ ID NO: 27 - DNA, 22 bp, Homo sapiens
tgggaaaggtgccgagacatga

SEQ ID NO: 28 - DNA, 20 bp, Homo sapiens
gctggccaggtcagtgcaac

SEQ ID NO: 29 - DNA, 21 bp, Homo sapiens
ttggaagcaaattgttggttt

SEQ ID NO: 30 - DNA, 24 bp, Homo sapiens
aagagattgtgattccacagagaa

SEQ ID NO: 31 - DNA, 26 bp, Homo sapiens
ttcagccatattaacatacattgaca

SEQ ID NO: 32 - DNA, 25 bp, Homo sapiens
ccatttgttaactggaatcattcat

SEQ ID NO: 33 - DNA, 25 bp, Homo sapiens
tggcaatttaacatgtgtattcttt

SEQ ID NO: 34 - DNA, 27 bp, Homo sapiens
cgaatacctaattatcctattctgaaa

SEQ ID NO: 35 - DNA, 22 bp, Homo sapiens
ccatttttctattcttggtggc

SEQ ID NO: 36 - DNA, 20 bp, Homo sapiens
gaaggagaggcttacctgct

SEQ ID NO: 37 - DNA, 21 bp, Homo sapiens
tgtcgcttttaaggaagtgct

SEQ ID NO: 38 - DNA, 21 bp, Homo sapiens
ccaatgcaaaaacttccatct

SEQ ID NO: 39 - DNA, 21 bp, Homo sapiens
gtcattcctctacatgggctg

SEQ ID NO: 40 - DNA, 22 bp, Homo sapiens
aaactgacaaacctatgggctg

SEQ ID NO: 41 - DNA, 20 bp, Homo sapiens
agactgtaagccctttgggg

SEQ ID NO: 42 - DNA, 20 bp, Homo sapiens
gataggagaacgccatccaa

SEQ ID NO: 43 - DNA, 20 bp, Homo sapiens
ccatactcccactttgtgcc

SEQ ID NO: 44 - DNA, 20 bp, Homo sapiens
cgacattgaagcaccatttg

SEQ ID NO: 45 - DNA, 20 bp, Homo sapiens
accagccaagagccgtgtgc

SEQ ID NO: 46 - DNA, 21 bp, Homo sapiens
gggcaagctaccctgcagcaa

SEQ ID NO: 47 - DNA, 24 bp, Homo sapiens
tcaaatgaagcgcaacagagggga

SEQ ID NO: 48 - DNA, 21 bp, Homo sapiens
agggcacggaggttggtgttg

SEQ ID NO: 49 - DNA, 20 bp, Homo sapiens
tttcctatacctccccaggc

SEQ ID NO: 50 - DNA, 20 bp, Homo sapiens
ctgtcaaaccctgagccaac

SEQ ID NO: 51 - DNA, 20 bp, Homo sapiens
ggcagtctcatgtcccctta

SEQ ID NO: 52 - DNA, 20 bp, Homo sapiens
tgtgtcgatgtcccttgaaa

SEQ ID NO: 53 - DNA, 25 bp, Homo sapiens
aaaggtaacttgtgtgtgattctga

SEQ ID NO: 54 - DNA, 23 bp, Homo sapiens
gaagccaaagtaaaacatgagga

SEQ ID NO: 55 - DNA, 20 bp, Homo sapiens
gcaactttgagtccttggct

SEQ ID NO: 56 - DNA, 20 bp, Homo sapiens
agctctcctgcagatatggc

SEQ ID NO: 57 - DNA, 20 bp, Homo sapiens
tccgggaatcctggggccaa

SEQ ID NO: 58 - DNA, 21 bp, Homo sapiens
gggtggttgtcctctgggggt

SEQ ID NO: 59 - DNA, 20 bp, Homo sapiens
gccggaggctagcagagcca

SEQ ID NO: 60 - DNA, 19 bp, Homo sapiens
gccgcagctcacgctccat

SEQ ID NO: 61 - DNA, 20 bp, Homo sapiens
agagtccctgtgcaaacacc

SEQ ID NO: 62 - DNA, 20 bp, Homo sapiens
cccttgaacccccttaccta

SEQ ID NO: 63 - DNA, 20 bp, Homo sapiens
aatgtggcagtcctctcagg

SEQ ID NO: 64 - DNA, 20 bp, Homo sapiens
gctgagctatgggtgaggaa

SEQ ID NO: 65 - DNA, 20 bp, Homo sapiens
aaatgcagggcacatgatct

SEQ ID NO: 66 - DNA, 20 bp, Homo sapiens
ggttgctgtctccgtgaaat

SEQ ID NO: 67 - DNA, 23 bp, Homo sapiens
tttgtggcttctctttacagctt

SEQ ID NO: 68 - DNA, 20 bp, Homo sapiens
caaacaacatgcagcagtga

SEQ ID NO: 69 - DNA, 20 bp, Homo sapiens
ggcagcggatcccatgtcgg

SEQ ID NO: 70 - DNA, 20 bp, Homo sapiens
gcacatctccacccccaggc

SEQ ID NO: 71 - DNA, 20 bp, Homo sapiens
agctttcggggttcggcagc

SEQ ID NO: 72 - DNA, 20 bp, Homo sapiens
tggagagcagcgaagggggt

SEQ ID NO: 73 - DNA, 20 bp, Homo sapiens
agagaagagcctgacgcaga

SEQ ID NO: 74 - DNA, 20 bp, Homo sapiens
agtgaatgccgacctttgaa

SEQ ID NO: 75 - DNA, 23 bp, Homo sapiens
tggcttgatctctcatacaaagg

SEQ ID NO: 76 - DNA, 21 bp, Homo sapiens
tcattgcatattttcagggaa

SEQ ID NO: 77 - DNA, 21 bp, Homo sapiens
tcccacccatgccttgctcca

SEQ ID NO: 78 - DNA, 27 bp, Homo sapiens
tgttctccatctcttggaggctgagac

SEQ ID NO: 79 - DNA, 25 bp, Homo sapiens
ggagcaacatgtttggccaagttcc

SEQ ID NO: 80 - DNA, 23 bp, Homo sapiens
tggtttctccaacggccaggact

SEQ ID NO: 81 - DNA, 23 bp, Homo sapiens
accccacatcttggcaacaacga

SEQ ID NO: 82 - DNA, 26 bp, Homo sapiens
tgtgggacattgttcgatgtgatgaa

SEQ ID NO: 83 - DNA, 25 bp, Homo sapiens
tgagtgcttcagcttctgatcccat

SEQ ID NO: 84 - DNA, 25 bp, Homo sapiens
ggtctcttgggaatcaaatgcccct

SEQ ID NO: 85 - DNA, 26 bp, Homo sapiens
tgggtccatttgaagcacagcagaag

SEQ ID NO: 86 - DNA, 22 bp, Homo sapiens
cattggcggggcttgaacctca

SEQ ID NO: 87 - DNA, 25 bp, Homo sapiens
tgccatgcaactgagaagtggttca

SEQ ID NO: 88 - DNA, 22 bp, Homo sapiens
tgcgacagcatagcacagtggg

SEQ ID NO: 89 - DNA, 21 bp, Homo sapiens
accgtcagccgaaggtctcgt

SEQ ID NO: 90 - DNA, 23 bp, Homo sapiens
gggactttgctttgcagggctga

SEQ ID NO: 91 - DNA, 22 bp, Homo sapiens
tctctttggcccagaggtgggt

SEQ ID NO: 92 - DNA, 24 bp, Homo sapiens
tgccagtattgtgaatgccacgct

SEQ ID NO: 93 - DNA, 20 bp, Homo sapiens
gcagtggctggtgggctacg

SEQ ID NO: 94 - DNA, 20 bp, Homo sapiens
ctcggacgacgtggcgaagg

SEQ ID NO: 95 - DNA, 23 bp, Homo sapiens
gctccaccctataattctgaacc

SEQ ID NO: 96 - DNA, 23 bp, Homo sapiens
ttttacaggagattggtacagcg

SEQ ID NO: 97 - DNA, 20 bp, Homo sapiens
gcctccgtgcactgaaccca

SEQ ID NO: 98 - DNA, 22 bp, Homo sapiens
gacagccaggtcctgtgagagc

SEQ ID NO: 99 - DNA, 22 bp, Homo sapiens
agcccagggagtggggaggata

SEQ ID NO: 100 - DNA, 22 bp, Homo sapiens
tgcctttcaaggaccccctcgg

SEQ ID NO: 101 - DNA, 21 bp, Homo sapiens
tggctgctggtgggtaactgc

SEQ ID NO: 102 - DNA, 21 bp, Homo sapiens
ccaggctgcccagggaagaac

SEQ ID NO: 103 - DNA, 22 bp, Homo sapiens
cccgacggcagatgatgacgac

SEQ ID NO: 104 - DNA, 21 bp, Homo sapiens
agacgctcagggtccatgcct

SEQ ID NO: 105 - DNA, 24 bp, Homo sapiens
aaaaacgtggtgttatttttgtga

SEQ ID NO: 106 - DNA, 20 bp, Homo sapiens
atgcatgcactctgcatttt

SEQ ID NO: 107 - DNA, 20 bp, Homo sapiens
attctacaaggcatgctggg

SEQ ID NO: 108 - DNA, 20 bp, Homo sapiens
gggagaacaaaacatgcagc

SEQ ID NO: 109 - DNA, 20 bp, Homo sapiens
agccaggacccacagccctc

SEQ ID NO: 110 - DNA, 20 bp, Homo sapiens
cgcggcggatggtttccact

SEQ ID NO: 111 - DNA, 23 bp, Homo sapiens
agcagcactgctaaccattccag

SEQ ID NO: 112 - DNA, 21 bp, Homo sapiens
agccgccagctcagggttaga

SEQ ID NO: 113 - DNA, 20 bp, Homo sapiens
ggaggcgggctttggccttt

SEQ ID NO: 114 - DNA, 20 bp, Homo sapiens
ggcctctacggggagccaga

SEQ ID NO: 115 - DNA, 20 bp, Homo sapiens
ttgcgcctgtgaccaccacc

SEQ ID NO: 116 - DNA, 20 bp, Homo sapiens
tggcctttcccgaccctgct

SEQ ID NO: 117 - DNA, 23 bp, Homo sapiens
acccagaaggggcagaagaacca

SEQ ID NO: 118 - DNA, 22 bp, Homo sapiens
gccatccaatgaggcaacccct

SEQ ID NO: 119 - DNA, 21 bp, Homo sapiens
ggaccgggcccctgggataaa

SEQ ID NO: 120 - DNA, 21 bp, Homo sapiens
tgcaagggcactgatgaggct

SEQ ID NO: 121 - DNA, 23 bp, Homo sapiens
tcccacgtgctgtcgatctttgg

SEQ ID NO: 122 - DNA, 25 bp, Homo sapiens
tgggcaccgataaacagattccaca

SEQ ID NO: 123 - DNA, 20 bp, Homo sapiens
gtgtggcgtcacttccggct

SEQ ID NO: 124 - DNA, 21 bp, Homo sapiens
tggttccgaggagttccgcac

SEQ ID NO: 125 - DNA, 19 bp, Homo sapiens
acgccagaaccttgtgagc

SEQ ID NO: 126 - DNA, 22 bp, Homo sapiens
gcatggatctccacctctactg

SEQ ID NO: 127 - DNA, 20 bp, Homo sapiens
tggaactgaggaagctggac

SEQ ID NO: 128 - DNA, 20 bp, Homo sapiens
ttcgatcatgaaaagggcac

SEQ ID NO: 129 - DNA, 22 bp, Homo sapiens
tgaaagaacaacttagggggtc

SEQ ID NO: 130 - DNA, 20 bp, Homo sapiens
tcaggaagcaaattccacaa

SEQ ID NO: 131 - DNA, 20 bp, Homo sapiens
ctattgcttcaccatggcct

SEQ ID NO: 132 - DNA, 20 bp, Homo sapiens
atcagggatccttggcaact

SEQ ID NO: 133 - DNA, 20 bp, Homo sapiens
tctgacgtccacctcctacc

SEQ ID NO: 134 - DNA, 20 bp, Homo sapiens
agccctgaaaaagtgtcagc

SEQ ID NO: 135 - DNA, 27 bp, Homo sapiens
catttctctacactaaggggtatgttc

SEQ ID NO: 136 - DNA, 21 bp, Homo sapiens
aaggtagggaaagggacaaga

SEQ ID NO: 137 - DNA, 20 bp, Homo sapiens
gagggcaatctttgccttta

SEQ ID NO: 138 - DNA, 20 bp, Homo sapiens
agaaagatggaggaggaccc

SEQ ID NO: 139 - DNA, 23 bp, Homo sapiens
tggcctcgagatgtatccctgcc

SEQ ID NO: 140 - DNA, 24 bp, Homo sapiens
tccatctcaggtaccaccgactgc

SEQ ID NO: 141 - DNA, 20 bp, Homo sapiens
tttccgcgcgcctctttggc

SEQ ID NO: 142 - DNA, 20 bp, Homo sapiens
acgcagagggcccgaccata

SEQ ID NO: 143 - DNA, 24 bp, Homo sapiens
tcgggaaccccctccttctcccat

SEQ ID NO: 144 - DNA, 22 bp, Homo sapiens
gcccagccgagccttcagtctt

SEQ ID NO: 145 - DNA, 24 bp, Homo sapiens
caggctgggcagatgctgtgatct

SEQ ID NO: 146 - DNA, 24 bp, Homo sapiens
ggagacggctcacagcctggaaac

SEQ ID NO: 147 - DNA, 24 bp, Homo sapiens
accctcctcccaaaaagcaagagc

SEQ ID NO: 148 - DNA, 21 bp, Homo sapiens
gtgcagaaatccgtgggggct

SEQ ID NO: 149 - DNA, 22 bp, Homo sapiens
ccaggtgatgctggggtgatgc

SEQ ID NO: 150 - DNA, 22 bp, Homo sapiens
gcaggccagagtgtctctcgga

SEQ ID NO: 151 - DNA, 23 bp, Homo sapiens
tgacccagaacttcctccccaca

SEQ ID NO: 152 - DNA, 23 bp, Homo sapiens
acctcagtgctggagaactggca

SEQ ID NO: 153 - DNA, 20 bp, Homo sapiens
ggccctcatcccaacagccc

SEQ ID NO: 154 - DNA, 21 bp, Homo sapiens
aatccctacccatccccgccc

SEQ ID NO: 155 - DNA, 27 bp, Homo sapiens
ggcccactttaaaagcacctgactagc

SEQ ID NO: 156 - DNA, 25 bp, Homo sapiens
aatcaagttgacaccctgcctctgc

SEQ ID NO: 157 - DNA, 21 bp, Homo sapiens
agctccgcatgtgaacccagc

SEQ ID NO: 158 - DNA, 21 bp, Homo sapiens
tgttggtggtggacctcggct

SEQ ID NO: 159 - DNA, 27 bp, Homo sapiens
cacgtgggagtatcctaaaactctgcc

SEQ ID NO: 160 - DNA, 24 bp, Homo sapiens
tcgctaaatgcagggtctgtccaa

SEQ ID NO: 161 - DNA, 23 bp, Homo sapiens
tggggacctcagatttccacccc

SEQ ID NO: 162 - DNA, 23 bp, Homo sapiens
tcaagtccagcgctcttccgagt

SEQ ID NO: 163 - DNA, 20 bp, Homo sapiens
tccagcctggggttagggca

SEQ ID NO: 164 - DNA, 20 bp, Homo sapiens
tgggagacccaaactgccgc

SEQ ID NO: 165 - DNA, 21 bp, Homo sapiens
gttcacatggaggctgcggct

SEQ ID NO: 166 - DNA, 21 bp, Homo sapiens
cacctttcctgggtcacgccg

SEQ ID NO: 167 - DNA, 20 bp, Homo sapiens
acctgcccctccactgcaca

SEQ ID NO: 168 - DNA, 21 bp, Homo sapiens
agcgcctttcaggtgccctct

SEQ ID NO: 169 - DNA, 20 bp, Homo sapiens
agggcaaggatcaactccat

SEQ ID NO: 170 - DNA, 26 bp, Homo sapiens
tcgtgtaatacatagggaatcaaatg

SEQ ID NO: 171 - DNA, 26 bp, Homo sapiens
tcagtgagtacaaaattgcagataca

SEQ ID NO: 172 - DNA, 23 bp, Homo sapiens
caaacaggcaattatgacacaga

SEQ ID NO: 173 - DNA, 27 bp, Homo sapiens
gccatgaagcatcagcagctcaaaagg

SEQ ID NO: 174 - DNA, 23 bp, Homo sapiens
tctgcttccttggctttggtgcc

SEQ ID NO: 175 - DNA, 25 bp, Homo sapiens
ccaaggatttggtggcatacccact

SEQ ID NO: 176 - DNA, 25 bp, Homo sapiens
agagctgtcaggaagaggagagtca

SEQ ID NO: 177 - DNA, 20 bp, Homo sapiens
taggcctcgtatcctgcact

SEQ ID NO: 178 - DNA, 20 bp, Homo sapiens
ccccagcttgtcttggatta

SEQ ID NO: 179 - DNA, 20 bp, Homo sapiens
gagcaaggtcttgttgcctc

SEQ ID NO: 180 - DNA, 20 bp, Homo sapiens
tcgtcgctgtcctaactcct

SEQ ID NO: 181 - DNA, 20 bp, Homo sapiens
aaggccacctccaagcgaca

SEQ ID NO: 182 - DNA, 21 bp, Homo sapiens
ctcggagaacttgccatcgcc

SEQ ID NO: 183 - DNA, 21 bp, Homo sapiens
tccgacacctcttcgcccctc

SEQ ID NO: 184 - DNA, 20 bp, Homo sapiens
tgggtcctggctctgccaca

SEQ ID NO: 185 - DNA, 23 bp, Homo sapiens
tccaagcactcactgccctcttg

SEQ ID NO: 186 - DNA, 22 bp, Homo sapiens
gcaggtgaggcaggcagaaact

SEQ ID NO: 187 - DNA, 24 bp, Homo sapiens
gcctcactgactaccttcagggca

SEQ ID NO: 188 - DNA, 26 bp, Homo sapiens
acccttctggttctcaaggagtttcc

SEQ ID NO: 189 - DNA, 23 bp, Homo sapiens
ccaagcactcactgccctcttgc

SEQ ID NO: 190 - DNA, 22 bp, Homo sapiens
aggcaggtgaggcaggcagaaa

SEQ ID NO: 191 - DNA, 25 bp, Homo sapiens
tgccactgtaaggttctctcagcct

SEQ ID NO: 192 - DNA, 26 bp, Homo sapiens
cagccaaagtgacttctgttcgtcca

SEQ ID NO: 193 - DNA, 21 bp, Homo sapiens
tccctccatgctccaggtcca

SEQ ID NO: 194 - DNA, 21 bp, Homo sapiens
tcccactacccccaacaccca

SEQ ID NO: 195 - DNA, 25 bp, Homo sapiens
cacagaaccccagaagtctcagcag

SEQ ID NO: 196 - DNA, 27 bp, Homo sapiens
caaaacctgtgcttgggttgaatctgt

SEQ ID NO: 197 - DNA, 26 bp, Homo sapiens
gggagaagacttatggaccccaagca

SEQ ID NO: 198 - DNA, 23 bp, Homo sapiens
tgcagagggtccaaagcaaagga

SEQ ID NO: 199 - DNA, 20 bp, Homo sapiens
ctaaccccaacagccacatc

SEQ ID NO: 200 - DNA, 20 bp, Homo sapiens
ccaccaccacttcctgagat

SEQ ID NO: 201 - DNA, 25 bp, Homo sapiens
ctgagccttaaagagatgaaataaa

SEQ ID NO: 202 - DNA, 20 bp, Homo sapiens
aggaattgggaggattttgc

SEQ ID NO: 203 - DNA, 23 bp, Homo sapiens
gcctcataactgttctacctcca

SEQ ID NO: 204 - DNA, 25 bp, Homo sapiens
catataaaacactggggaaaatcac

SEQ ID NO: 205 - DNA, 20 bp, Homo sapiens
caagctccccgactcaagta

SEQ ID NO: 206 - DNA, 20 bp, Homo sapiens
cagagagacaggcaagccat

SEQ ID NO: 207 - DNA, 27 bp, Homo sapiens
actgttgcttcttcttctaccagtggg

SEQ ID NO: 208 - DNA, 27 bp, Homo sapiens
ttgtcagtttggaaagtcacagggagt

SEQ ID NO: 209 - DNA, 25 bp, Homo sapiens
gactccctggaccctgagaagctat

SEQ ID NO: 210 - DNA, 25 bp, Homo sapiens
aggaaacctgcaagccctgatttga

SEQ ID NO: 211 - DNA, 20 bp, Homo sapiens
accggactggaggccgtgag

SEQ ID NO: 212 - DNA, 20 bp, Homo sapiens
taccacagcccctggcagca

SEQ ID NO: 213 - DNA, 20 bp, Homo sapiens
cctacccgtggggcccgtaa

SEQ ID NO: 214 - DNA, 20 bp, Homo sapiens
cgcagcgctcctgctctgac

SEQ ID NO: 215 - DNA, 20 bp, Homo sapiens
cccgcgtctgggttctcagc

SEQ ID NO: 216 - DNA, 20 bp, Homo sapiens
ccccctccgagctccgactt

SEQ ID NO: 217 - DNA, 20 bp, Homo sapiens
agcgccgagtgcgagtttgt

SEQ ID NO: 218 - DNA, 20 bp, Homo sapiens
cgggccccaggcgtactaga

SEQ ID NO: 219 - DNA, 20 bp, Homo sapiens
ggggaaccgagtgtcgccca

SEQ ID NO: 220 - DNA, 20 bp, Homo sapiens
aggggcttcccgaggctgag

SEQ ID NO: 221 - DNA, 20 bp, Homo sapiens
ctggtggcgctctcgttggc

SEQ ID NO: 222 - DNA, 20 bp, Homo sapiens
ccgcttgatgcccagcaggt

SEQ ID NO: 223 - DNA, 21 bp, Homo sapiens
atgtgtctgccccctctatgt

SEQ ID NO: 224 - DNA, 21 bp, Homo sapiens
acaagaaacgaagccagggac

SEQ ID NO: 225 - DNA, 20 bp, Homo sapiens
gtagcgcattgcggcgacct

SEQ ID NO: 226 - DNA, 20 bp, Homo sapiens
agtccttgggttccgcggct

SEQ ID NO: 227 - DNA, 26 bp, Homo sapiens
gtagctccatattggacatccccaga

SEQ ID NO: 228 - DNA, 21 bp, Homo sapiens
gggtcccactggaagggcatt

SEQ ID NO: 229 - DNA, 22 bp, Homo sapiens
tctgcgagtataggaagctctc

SEQ ID NO: 230 - DNA, 19 bp, Homo sapiens
ccctgcatgaagactgggt

SEQ ID NO: 231 - DNA, 20 bp, Homo sapiens
tgcaaagcactggcatttta

SEQ ID NO: 232 - DNA, 21 bp, Homo sapiens
ggggcttctcattgcatttat

SEQ ID NO: 233 - DNA, 20 bp, Homo sapiens
tgaactgtcacagagcaggc

SEQ ID NO: 234 - DNA, 20 bp, Homo sapiens
gccaggctgttctgcttatg

SEQ ID NO: 235 - DNA, 20 bp, Homo sapiens
tgccagagagtacagaaggg

SEQ ID NO: 236 - DNA, 19 bp, Homo sapiens
ccggagggacttgaagact

SEQ ID NO: 237 - DNA, 20 bp, Homo sapiens
acacagccaagccctgctgc

SEQ ID NO: 238 - DNA, 20 bp, Homo sapiens
gccccaggaggcagacaacg

SEQ ID NO: 239 - DNA, 20 bp, Homo sapiens
ctgtgcagggccgggatagc

SEQ ID NO: 240 - DNA, 20 bp, Homo sapiens
ccacagcccagcaggagcac

SEQ ID NO: 241 - DNA, 20 bp, Homo sapiens
attctgggcaccggcgcttg

SEQ ID NO: 242 - DNA, 20 bp, Homo sapiens
cggccggggttctacccaga

SEQ ID NO: 243 - DNA, 26 bp, Homo sapiens
gctgagctgatcatacactgacctga

SEQ ID NO: 244 - DNA, 27 bp, Homo sapiens
ggggaaagtgagcatgaaaagaactcc

SEQ ID NO: 245 - DNA, 21 bp, Homo sapiens
atggtgccttccctccctcca

SEQ ID NO: 246 - DNA, 27 bp, Homo sapiens
gtgcatatctcatcactccacagcagc

SEQ ID NO: 247 - DNA, 26 bp, Homo sapiens
acagtgatgaacgaagagatgcaccc

SEQ ID NO: 248 - DNA, 22 bp, Homo sapiens
cctctcctgacccctgaggcac

SEQ ID NO: 249 - DNA, 20 bp, Homo sapiens
cttccgcatggtcatctcaa

SEQ ID NO: 250 - DNA, 25 bp, Homo sapiens
cttgtccaaggcgttctatttcttc

SEQ ID NO: 251 - DNA, 20 bp, Homo sapiens
atgtggcaacctgttcttcc

SEQ ID NO: 252 - DNA, 24 bp, Homo sapiens
ttgggtttagagagacaacacaga

SEQ ID NO: 253 - DNA, 20 bp, Homo sapiens
gtgtgccaaactaccgttcc

SEQ ID NO: 254 - DNA, 20 bp, Homo sapiens
atgttgggggaatgttgatg

SEQ ID NO: 255 - DNA, 20 bp, Homo sapiens
tgcccccattgcccacttgc

SEQ ID NO: 256 - DNA, 20 bp, Homo sapiens
gaccccctcctagcctgccc

SEQ ID NO: 257 - DNA, 20 bp, Homo sapiens
gcgaggggtccaggctctct

SEQ ID NO: 258 - DNA, 20 bp, Homo sapiens
tgaggcctgctggggacagg

SEQ ID NO: 259 - DNA, 22 bp, Homo sapiens
cacattcaagcaataacccacg

SEQ ID NO: 260 - DNA, 22 bp, Homo sapiens
cccatgtctagctcatcctcat

SEQ ID NO: 261 - DNA, 20 bp, Homo sapiens
aaacctacagcatcccacca

SEQ ID NO: 262 - DNA, 20 bp, Homo sapiens
gctgcaacacgtcacaacat

SEQ ID NO: 263 - DNA, 21 bp, Homo sapiens
ctgcatgcaagttatgacagg

SEQ ID NO: 264 - DNA, 21 bp, Homo sapiens
acgactctcggttgctaaatg

SEQ ID NO: 265 - DNA, 20 bp, Homo sapiens
aatcgctgaggacccgggca

SEQ ID NO: 266 - DNA, 20 bp, Homo sapiens
ggaggcaggcagtgggcaag

SEQ ID NO: 267 - DNA, 22 bp, Homo sapiens
tgcacccatccctggtgaacct

SEQ ID NO: 268 - DNA, 22 bp, Homo sapiens
tgggctccttgcagagaggctt

SEQ ID NO: 269 - DNA, 20 bp, Homo sapiens
ggcgacgactacaagagcag

SEQ ID NO: 270 - DNA, 21 bp, Homo sapiens
cggaatgcacacgtacaggtt

SEQ ID NO: 271 - DNA, 21 bp, Homo sapiens
ggcgaaggtaggctggcagat

SEQ ID NO: 272 - DNA, 23 bp, Homo sapiens
tgacggcagggtgaagagagact

SEQ ID NO: 273 - DNA, 20 bp, Homo sapiens
ttccttcggtcgctgccacg

SEQ ID NO: 274 - DNA, 20 bp, Homo sapiens
tcatggttggcggagagcgg

SEQ ID NO: 275 - DNA, 23 bp, Homo sapiens
tgttggcaaaggaacgtcctgct

SEQ ID NO: 276 - DNA, 24 bp, Homo sapiens
agtcgtccagtttcccactaccct

SEQ ID NO: 277 - DNA, 21 bp, Homo sapiens
ctatccgtcctacctctcgca

SEQ ID NO: 278 - DNA, 21 bp, Homo sapiens
acatgcagcagacattctcct

SEQ ID NO: 279 - DNA, 22 bp, Homo sapiens
atgtttaactcggtcaacctgg

SEQ ID NO: 280 - DNA, 22 bp, Homo sapiens
gcatgtagtaagtgcaactggg

SEQ ID NO: 281 - DNA, 23 bp, Homo sapiens
ttcaaatcacgcatctctactcc

SEQ ID NO: 282 - DNA, 20 bp, Homo sapiens
tgcacatgtacacacgcact

SEQ ID NO: 283 - DNA, 20 bp, Homo sapiens
agtagggagatgggattggg

SEQ ID NO: 284 - DNA, 20 bp, Homo sapiens
ccctccaggtggaaagaaac

SEQ ID NO: 285 - DNA, 21 bp, Homo sapiens
ctcgctcatctctcacgacaa

SEQ ID NO: 286 - DNA, 21 bp, Homo sapiens
gacggaaaatcgctacagtcc

SEQ ID NO: 287 - DNA, 23 bp, Homo sapiens
tctaagaccaaagggatgtgttt

SEQ ID NO: 288 - DNA, 24 bp, Homo sapiens
aatgttctatgcatttcttcatcc

SEQ ID NO: 289 - DNA, 23 bp, Homo sapiens
cacgtaaatttcctcacatcaca

SEQ ID NO: 290 - DNA, 23 bp, Homo sapiens
tttggtctgtgttttctcctttc

SEQ ID NO: 291 - DNA, 25 bp, Homo sapiens
aaaggagaaagaaaatcaacaaatg

SEQ ID NO: 292 - DNA, 20 bp, Homo sapiens
ggaccatcaccctaaccctt

SEQ ID NO: 293 - DNA, 20 bp, Homo sapiens
agggaaggtcaagagggaga

SEQ ID NO: 294 - DNA, 20 bp, Homo sapiens
agggaaggtcaagagggaga

SEQ ID NO: 295 - DNA, 20 bp, Homo sapiens
accgaggcggctcttggaca

SEQ ID NO: 296 - DNA, 20 bp, Homo sapiens
cggcctgctccaggctcatt

SEQ ID NO: 297 - DNA, 21 bp, Homo sapiens
tgcacctgaccagtcgtccca

SEQ ID NO: 298 - DNA, 21 bp, Homo sapiens
ctggagccacactccctggct

SEQ ID NO: 299 - DNA, 23 bp, Homo sapiens
ctggaaaaagtcccacttctgaa

SEQ ID NO: 300 - DNA, 21 bp, Homo sapiens
aaaagactcgcaggagcaaaa

SEQ ID NO: 301 - DNA, 20 bp, Homo sapiens
ctctcccattcaccctgtgt

SEQ ID NO: 302 - DNA, 20 bp, Homo sapiens
ccccgaccctctaggttaag

SEQ ID NO: 303 - DNA, 20 bp, Homo sapiens
agaactcctcccacccaact

SEQ ID NO: 304 - DNA, 20 bp, Homo sapiens
cccactctcatctcacagca

SEQ ID NO: 305 - DNA, 21 bp, Homo sapiens
gcagggctaaggaattactgg

SEQ ID NO: 306 - DNA, 22 bp, Homo sapiens
ccctaaggtattacccacaggc

SEQ ID NO: 307 - DNA, 23 bp, Homo sapiens
tcatgaatgtttgaaaggaacaa

SEQ ID NO: 308 - DNA, 20 bp, Homo sapiens
taagacccatggcaaagagc

SEQ ID NO: 309 - DNA, 20 bp, Homo sapiens
agttagacccagggtgtgga

SEQ ID NO: 310 - DNA, 22 bp, Homo sapiens
caaaaccaaaacagcacagaga

SEQ ID NO: 311 - DNA, 22 bp, Homo sapiens
tgaagttttcagatcagtggca

SEQ ID NO: 312 - DNA, 20 bp, Homo sapiens
acaaagcagggtcatagcca

SEQ ID NO: 313 - DNA, 20 bp, Homo sapiens
ggaggcggcagttccaccat

SEQ ID NO: 314 - DNA, 22 bp, Homo sapiens
ggaccgagagctagcagacgca

SEQ ID NO: 315 - DNA, 24 bp, Homo sapiens
ccgagcctgattcctagtcctgct

SEQ ID NO: 316 - DNA, 22 bp, Homo sapiens
tggatgtgctggccatggttcc

SEQ ID NO: 317 - DNA, 24 bp, Homo sapiens
tgcagtgtccctgaatggcaagtg

SEQ ID NO: 318 - DNA, 26 bp, Homo sapiens
aggcaaagagagcagagaaagcagtg

SEQ ID NO: 319 - DNA, 20 bp, Homo sapiens
tgctgccgccagctctcagt

SEQ ID NO: 320 - DNA, 20 bp, Homo sapiens
tggaggccaggggaggacaa

SEQ ID NO: 321 - DNA, 22 bp, Homo sapiens
ctgtctgaggagggcagagcca

SEQ ID NO: 322 - DNA, 22 bp, Homo sapiens
ccctgggaaccagcagtctgga

SEQ ID NO: 323 - DNA, 24 bp, Homo sapiens
gcagctgccttaccttccagatga

SEQ ID NO: 324 - DNA, 20 bp, Homo sapiens
accctcctctgccccagcat

SEQ ID NO: 325 - DNA, 21 bp, Homo sapiens
gccccctcctattccagccca

SEQ ID NO: 326 - DNA, 20 bp, Homo sapiens
tgtggcggactccccatcct

SEQ ID NO: 327 - DNA, 18 bp, Homo sapiens
aagggccaggaccagaac

SEQ ID NO: 328 - DNA, 20 bp, Homo sapiens
tcaagagcagaggaggaagg

SEQ ID NO: 329 - DNA, 20 bp, Homo sapiens
ctgtgtgatcccccacttct

SEQ ID NO: 330 - DNA, 20 bp, Homo sapiens
ctttctagggtggcctttcc

SEQ ID NO: 331 - DNA, 27 bp, Homo sapiens
cctggtagtcaatttgttgtcccgagg

SEQ ID NO: 332 - DNA, 22 bp, Homo sapiens
tggggaagagctcaggcaagac

SEQ ID NO: 333 - DNA, 20 bp, Homo sapiens
gtctgcggcggcagctgtaa

SEQ ID NO: 334 - DNA, 20 bp, Homo sapiens
gtggcgcgaggtactggctg

SEQ ID NO: 335 - DNA, 20 bp, Homo sapiens
tggcgatctctgcgcctcca

SEQ ID NO: 336 - DNA, 20 bp, Homo sapiens
agtgcccaccaccacgttgc

SEQ ID NO: 337 - DNA, 22 bp, Homo sapiens
gcagaaccagcttcacccccag

SEQ ID NO: 338 - DNA, 21 bp, Homo sapiens
aggtgaccttggctgtcccct

SEQ ID NO: 339 - DNA, 22 bp, Homo sapiens
tgcattgcacccacacacaacg

SEQ ID NO: 340 - DNA, 25 bp, Homo sapiens
gttgtgtgagccaggacagaaacca

SEQ ID NO: 341 - DNA, 21 bp, Homo sapiens
tggtggtaatggggaggggca

SEQ ID NO: 342 - DNA, 24 bp, Homo sapiens
ccttgcctctctgggtcatcctct

SEQ ID NO: 343 - DNA, 22 bp, Homo sapiens
gcctacatctgccactcagccg

SEQ ID NO: 344 - DNA, 20 bp, Homo sapiens
atccagggggtgcggtgtct

SEQ ID NO: 345 - DNA, 24 bp, Homo sapiens
tgtggaagagccgtgggattctct

SEQ ID NO: 346 - DNA, 21 bp, Homo sapiens
gggtgaaccacagggcgatgg

SEQ ID NO: 347 - DNA, 21 bp, Homo sapiens
agttccgggactggtgcttgc

SEQ ID NO: 348 - DNA, 22 bp, Homo sapiens
tcggcgatccgctggtatttgc

SEQ ID NO: 349 - DNA, 20 bp, Homo sapiens
agccggcgagaaaggcaagt

SEQ ID NO: 350 - DNA, 20 bp, Homo sapiens
tggcccatcttgggttcccg

SEQ ID NO: 351 - DNA, 21 bp, Homo sapiens
ttgcctcctccaatgttcctc

SEQ ID NO: 352 - DNA, 23 bp, Homo sapiens
cagttagcatcaatttgagccac

SEQ ID NO: 353 - DNA, 20 bp, Homo sapiens
gatggctcaggctgaagaac

SEQ ID NO: 354 - DNA, 20 bp, Homo sapiens
caccaggataaaagcaggga

SEQ ID NO: 355 - DNA, 20 bp, Homo sapiens
cgtagagctgtggctaaggg

SEQ ID NO: 356 - DNA, 20 bp, Homo sapiens
tattgccttgttgcttgctg

SEQ ID NO: 357 - DNA, 20 bp, Homo sapiens
cagatacaagcgctgatgga

SEQ ID NO: 358 - DNA, 20 bp, Homo sapiens
ttgtgctttcagttctcgga

SEQ ID NO: 359 - DNA, 20 bp, Homo sapiens
gattggcacagtcagtccct

SEQ ID NO: 360 - DNA, 20 bp, Homo sapiens
caaagcgctgccttaaactt

SEQ ID NO: 361 - DNA, 26 bp, Homo sapiens
agagtcccttaccatgacccacagat

SEQ ID NO: 362 - DNA, 22 bp, Homo sapiens
gggcttctggttctggtgatgc

SEQ ID NO: 363 - DNA, 20 bp, Homo sapiens
cgggccgaggctttgctagg

SEQ ID NO: 364 - DNA, 20 bp, Homo sapiens
gagcccccgctactccgaca

SEQ ID NO: 365 - DNA, 22 bp, Homo sapiens
gctcatcctttacaccaactcc

SEQ ID NO: 366 - DNA, 26 bp, Homo sapiens
ccaagtacttctcatttaagacagag

SEQ ID NO: 367 - DNA, 20 bp, Homo sapiens
gaaaaggaataagccctgcc

SEQ ID NO: 368 - DNA, 21 bp, Homo sapiens
agacaggtcaactaggggaaa

SEQ ID NO: 369 - DNA, 21 bp, Homo sapiens
gtcacatggcagcctggccta

SEQ ID NO: 370 - DNA, 21 bp, Homo sapiens
agcccaaactcccctccctgt

SEQ ID NO: 371 - DNA, 27 bp, Homo sapiens
tggaatctgttgtttccccctaagttg

SEQ ID NO: 372 - DNA, 24 bp, Homo sapiens
ggaaccatgtaacccaggccaaga

SEQ ID NO: 373 - DNA, 20 bp, Homo sapiens
acggccgaccagctggagat

SEQ ID NO: 374 - DNA, 20 bp, Homo sapiens
tcgtcgtccgggtcgcagat

SEQ ID NO: 375 - DNA, 20 bp, Homo sapiens
tccgcaacccttgccgcatc

SEQ ID NO: 376 - DNA, 20 bp, Homo sapiens
cgcgtccttgctcgggtgtt

SEQ ID NO: 377 - DNA, 21 bp, Homo sapiens
cctccactcggaaggactatc

SEQ ID NO: 378 - DNA, 21 bp, Homo sapiens
tcggttgttgctgatctgtct

SEQ ID NO: 379 - DNA, 21 bp, Homo sapiens
ccacagcaaacctcctcacag

SEQ ID NO: 380 - DNA, 21 bp, Homo sapiens
gcaggatagtccttccgagtg

SEQ ID NO: 381 - DNA, 22 bp, Homo sapiens
cctatgatgcaagccatttcca

SEQ ID NO: 382 - DNA, 21 bp, Homo sapiens
acacgcagaccctcaacaaag

SEQ ID NO: 383 - DNA, 21 bp, Homo sapiens
cacaaacaaagtgtctgctgc

SEQ ID NO: 384 - DNA, 21 bp, Homo sapiens
gaatggtgcttggatttgaga

SEQ ID NO: 385 - DNA, 20 bp, Homo sapiens
cacccatgacaaactctcca

SEQ ID NO: 386 - DNA, 20 bp, Homo sapiens
ccgtccagattgctttgttt

SEQ ID NO: 387 - DNA, 20 bp, Homo sapiens
gggatcagtttacctgccaa

SEQ ID NO: 388 - DNA, 20 bp, Homo sapiens
ggcctggattcctgatcttt

SEQ ID NO: 389 - DNA, 20 bp, Homo sapiens
ggaacttgcttccctctcct

SEQ ID NO: 390 - DNA, 20 bp, Homo sapiens
gccaatgctacaaacccagt

SEQ ID NO: 391 - DNA, 20 bp, Homo sapiens
tatgcctttggggagtatgg

SEQ ID NO: 392 - DNA, 20 bp, Homo sapiens
acaatttcagggaggcttgg

SEQ ID NO: 393 - DNA, 20 bp, Homo sapiens
gtgcttgcaaacccttccta

SEQ ID NO: 394 - DNA, 20 bp, Homo sapiens
ccctcactccaaaactcagc

SEQ ID NO: 395 - DNA, 20 bp, Homo sapiens
aacgctttgggaagaagtga

SEQ ID NO: 396 - DNA, 20 bp, Homo sapiens
ttgacacttttgggtgtgga

SEQ ID NO: 397 - DNA, 19 bp, Homo sapiens
cttcctgtttcagccccag

SEQ ID NO: 398 - DNA, 20 bp, Homo sapiens
ctccaggatccctgggtatt

SEQ ID NO: 399 - DNA, 27 bp, Homo sapiens
gctaaggaagttgctgcagaagctcag

SEQ ID NO: 400 - DNA, 26 bp, Homo sapiens
taccctaatgggtcaagtccctggtc

SEQ ID NO: 401 - DNA, 24 bp, Homo sapiens
gggcatttggatgccctaactgct

SEQ ID NO: 402 - DNA, 27 bp, Homo sapiens
caccatccaccctccatcaacaaacaa

SEQ ID NO: 403 - DNA, 20 bp, Homo sapiens
ccgcctccggaatgcacagg

SEQ ID NO: 404 - DNA, 20 bp, Homo sapiens
tggccaccacagactccccc

SEQ ID NO: 405 - DNA, 20 bp, Homo sapiens
gcctcgccacacatgccctt

SEQ ID NO: 406 - DNA, 20 bp, Homo sapiens
tgctgccggagaggctgtca

SEQ ID NO: 407 - DNA, 20 bp, Homo sapiens
tgaggagccttcacctctgt

SEQ ID NO: 408 - DNA, 20 bp, Homo sapiens
gtgggtgcttcacacctttt

SEQ ID NO: 409 - DNA, 20 bp, Homo sapiens
tattccctgctgcctacctg

SEQ ID NO: 410 - DNA, 20 bp, Homo sapiens
ctgctaagtgtcctgccctc

SEQ ID NO: 411 - DNA, 27 bp, Homo sapiens
aaacttatcattccagtatgcatcttt

SEQ ID NO: 412 - DNA, 27 bp, Homo sapiens
tgcatttcttaacatgtatagcactct

SEQ ID NO: 413 - DNA, 22 bp, Homo sapiens
atggttaaatgcataccttccc

SEQ ID NO: 414 - DNA, 23 bp, Homo sapiens
tgcacattctaagaagggtcatt

SEQ ID NO: 415 - DNA, 20 bp, Homo sapiens
gagctggaagctcaactggt

SEQ ID NO: 416 - DNA, 20 bp, Homo sapiens
ctttcatgggagggagatca

SEQ ID NO: 417 - DNA, 20 bp, Homo sapiens
gattgctgggggtttgtaga

SEQ ID NO: 418 - DNA, 20 bp, Homo sapiens
gagccctagtttcctgggac

SEQ ID NO: 419 - DNA, 23 bp, Homo sapiens
tgatgccgaggaactattcatct

SEQ ID NO: 420 - DNA, 21 bp, Homo sapiens
tttcttctcgtgcagtgtcac

SEQ ID NO: 421 - DNA, 20 bp, Homo sapiens
ggacaggaagtggacacgaa

SEQ ID NO: 422 - DNA, 20 bp, Homo sapiens
tcttgctgtcccttcctaga

SEQ ID NO: 423 - DNA, 20 bp, Homo sapiens
ccgctgcttccctgatgggc

SEQ ID NO: 424 - DNA, 21 bp, Homo sapiens
tggggaagctctttcggccct

SEQ ID NO: 425 - DNA, 24 bp, Homo sapiens
ccgttccagagactcatccagccg

SEQ ID NO: 426 - DNA, 25 bp, Homo sapiens
atcggcttggtggggaaatactccg

SEQ ID NO: 427 - DNA, 27 bp, Homo sapiens
gagtaagataatctggactggcccccg

SEQ ID NO: 428 - DNA, 27 bp, Homo sapiens
tccatggtctcttgatcaaatggggca

SEQ ID NO: 429 - DNA, 20 bp, Homo sapiens
tccgcggaccgggctttagg

SEQ ID NO: 430 - DNA, 20 bp, Homo sapiens
tttgccaccgccatggctcc

SEQ ID NO: 431 - DNA, 20 bp, Homo sapiens
tggataaatcccgaaaagga

SEQ ID NO: 432 - DNA, 20 bp, Homo sapiens
tccacaatggtttgaggagc

SEQ ID NO: 433 - DNA, 20 bp, Homo sapiens
tcatcctaaaacaggtcgca

SEQ ID NO: 434 - DNA, 20 bp, Homo sapiens
cggcagatgaagcttatggt

SEQ ID NO: 435 - DNA, 19 bp, Homo sapiens
gctgtccacctcgcctttt

SEQ ID NO: 436 - DNA, 21 bp, Homo sapiens
tcctgaaacgtccgtgtcttg

SEQ ID NO: 437 - DNA, 20 bp, Homo sapiens
caccgaagtgacccaaatgc

SEQ ID NO: 438 - DNA, 21 bp, Homo sapiens
gtctcggacaggactatcctt

SEQ ID NO: 439 - DNA, 23 bp, Homo sapiens
aagaggatcaccccaggaacgct

SEQ ID NO: 440 - DNA, 22 bp, Homo sapiens
acagccccaagctgggtcttca

SEQ ID NO: 441 - DNA, 20 bp, Homo sapiens
tgacacacgcccggcacatt

SEQ ID NO: 442 - DNA, 23 bp, Homo sapiens
tcccccatggacatccaagctgt

SEQ ID NO: 443 - DNA, 20 bp, Homo sapiens
agctcaacggggcagaggga

SEQ ID NO: 444 - DNA, 20 bp, Homo sapiens
tgggagggaggatggtgcgt

SEQ ID NO: 445 - DNA, 20 bp, Homo sapiens
tgcgcagctgacccacgatg

SEQ ID NO: 446 - DNA, 21 bp, Homo sapiens
cagctcgcggacctggaactc

SEQ ID NO: 447 - DNA, 21 bp, Homo sapiens
tgggtctcttgtggagagcaa

SEQ ID NO: 448 - DNA, 21 bp, Homo sapiens
tccgcatcatggaatcaatgg

SEQ ID NO: 449 - DNA, 21 bp, Homo sapiens
acatcgccaaacctcgcccag

SEQ ID NO: 450 - DNA, 20 bp, Homo sapiens
cgctactctaggcgccacgg

SEQ ID NO: 451 - DNA, 20 bp, Homo sapiens
gggcttgagcctgacagcga

SEQ ID NO: 452 - DNA, 20 bp, Homo sapiens
tcctcctgccacttcccgca

SEQ ID NO: 453 - DNA, 24 bp, Homo sapiens
catcgtcttcagcaaggatcagtt

SEQ ID NO: 454 - DNA, 19 bp, Homo sapiens
tgccgtccacctcaatgtc

SEQ ID NO: 455 - DNA, 21 bp, Homo sapiens
atggctgcaatccgaaagaag

SEQ ID NO: 456 - DNA, 21 bp, Homo sapiens
acagtagggacgtagacctcc

SEQ ID NO: 457 - DNA, 20 bp, Homo sapiens
agccattgttgcacattttg

SEQ ID NO: 458 - DNA, 20 bp, Homo sapiens
ccccatgtactggacacaga

SEQ ID NO: 459 - DNA, 20 bp, Homo sapiens
ggtaagagttccataccggc

SEQ ID NO: 460 - DNA, 22 bp, Homo sapiens
ccctacccagaaggtgtatgaa

SEQ ID NO: 461 - DNA, 21 bp, Homo sapiens
ttaacaggggaaaagggaaga

SEQ ID NO: 462 - DNA, 21 bp, Homo sapiens
cactagcagaacctcaaggga

SEQ ID NO: 463 - DNA, 25 bp, Homo sapiens
tgtgtttatgtggaatgtttaagga

SEQ ID NO: 464 - DNA, 23 bp, Homo sapiens
agcagcacaattatttcatgtca

SEQ ID NO: 465 - DNA, 22 bp, Homo sapiens
tgctgtgggttctcggtgttca

SEQ ID NO: 466 - DNA, 27 bp, Homo sapiens
acactgggaaaccatctatcagcaagc

SEQ ID NO: 467 - DNA, 23 bp, Homo sapiens
tggcattctcgcctatacccact

SEQ ID NO: 468 - DNA, 25 bp, Homo sapiens
acccaattaaggacgttgtgccagt

SEQ ID NO: 469 - DNA, 21 bp, Homo sapiens
gtccagcagtaaagcgattgt

SEQ ID NO: 470 - DNA, 21 bp, Homo sapiens
ggggtaagccttttctgtgag

SEQ ID NO: 471 - DNA, 21 bp, Homo sapiens
gcattgctttgccttatcatc

SEQ ID NO: 472 - DNA, 22 bp, Homo sapiens
aagacaactgaaacaaatggca

SEQ ID NO: 473 - DNA, 21 bp, Homo sapiens
tgttttgctgttgttgctttg

SEQ ID NO: 474 - DNA, 21 bp, Homo sapiens
acaggtacaggctatgagggg

SEQ ID NO: 475 - DNA, 19 bp, Homo sapiens
gtgtcctgagtgggtctga

SEQ ID NO: 476 - DNA, 25 bp, Homo sapiens
cctttgtctcacttcccttatcaag

SEQ ID NO: 477 - DNA, 19 bp, Homo sapiens
cgcagcgtgactgtgagtt

SEQ ID NO: 478 - DNA, 24 bp, Homo sapiens
tccctcaaaccaacttgtcaacag

SEQ ID NO: 479 - DNA, 21 bp, Homo sapiens
gacacgctggatctcacctac

SEQ ID NO: 480 - DNA, 21 bp, Homo sapiens
aactggtcctatgaggtcgca

SEQ ID NO: 481 - DNA, 27 bp, Homo sapiens
ccttgctttccaagttgttgcagcatt

SEQ ID NO: 482 - DNA, 24 bp, Homo sapiens
agagccggtcaaagtcttggaggt

SEQ ID NO: 483 - DNA, 20 bp, Homo sapiens
agcccgtgtctgagggggtg

SEQ ID NO: 484 - DNA, 20 bp, Homo sapiens
cggcccggctgaagtttccc

SEQ ID NO: 485 - DNA, 23 bp, Homo sapiens
agaccaacagcagatagtttcag

SEQ ID NO: 486 - DNA, 23 bp, Homo sapiens
gcacttactttgtgctataccct

SEQ ID NO: 487 - DNA, 22 bp, Homo sapiens
agcagtcaagatttggttggtg

SEQ ID NO: 488 - DNA, 23 bp, Homo sapiens
cctgagttgatgctgattctacc

SEQ ID NO: 489 - DNA, 25 bp, Homo sapiens
agctggttaggtagagggagttgtc

SEQ ID NO: 490 - DNA, 22 bp, Homo sapiens
ggttcaccaagaggttgtcaga

SEQ ID NO: 491 - DNA, 23 bp, Homo sapiens
ttccaatgttgtgattctgacca

SEQ ID NO: 492 - DNA, 23 bp, Homo sapiens
ttccaatgttgtgattctgacca

SEQ ID NO: 493 - DNA, 20 bp, Homo sapiens
tggcccaggaggaatattta

SEQ ID NO: 494 - DNA, 20 bp, Homo sapiens
gcattctcggtctgaagagg

SEQ ID NO: 495 - DNA, 20 bp, Homo sapiens
tcagggggtgtcggtagtag

SEQ ID NO: 496 - DNA, 20 bp, Homo sapiens
gtttctgctgcctcttcacc

SEQ ID NO: 497 - DNA, 20 bp, Homo sapiens
gggcctgggtctggtgtcca

SEQ ID NO: 498 - DNA, 20 bp, Homo sapiens
acagcacccaccagtccagc

SEQ ID NO: 499 - DNA, 21 bp, Homo sapiens
cggtccccaataccctccccc

SEQ ID NO: 500 - DNA, 20 bp, Homo sapiens
agcaggcaaagccgccacaa

SEQ ID NO: 501 - DNA, 20 bp, Homo sapiens
gctgggaactgtactgcctg

SEQ ID NO: 502 - DNA, 20 bp, Homo sapiens
gtccccaccaccaatggttt

SEQ ID NO: 503 - DNA, 21 bp, Homo sapiens
agttccttcccagtcacaacc

SEQ ID NO: 504 - DNA, 21 bp, Homo sapiens
aattaccaaagggtacgtggc

SEQ ID NO: 505 - DNA, 22 bp, Homo sapiens
agttgagctcttgaggatcaga

SEQ ID NO: 506 - DNA, 23 bp, Homo sapiens
tgacatagagcaaaatgaacaca

SEQ ID NO: 507 - DNA, 22 bp, Homo sapiens
ttgagtcacaagagaagcctgt

SEQ ID NO: 508 - DNA, 25 bp, Homo sapiens
agctggatctataaatcattttcca

SEQ ID NO: 509 - DNA, 21 bp, Homo sapiens
aagtggatactttgccttggg

SEQ ID NO: 510 - DNA, 20 bp, Homo sapiens
ctatacatcagggccttgcc

SEQ ID NO: 511 - DNA, 24 bp, Homo sapiens
ggaaaaaccttcccctccacagcc

SEQ ID NO: 512 - DNA, 25 bp, Homo sapiens
ttcaggtgagccagtcttctttgga

SEQ ID NO: 513 - DNA, 24 bp, Homo sapiens
ccacctcaactcattgactccgcc

SEQ ID NO: 514 - DNA, 23 bp, Homo sapiens
ggtgtctggccaccatcttcagc

SEQ ID NO: 515 - DNA, 20 bp, Homo sapiens
ccagttggtgactgggaaag

SEQ ID NO: 516 - DNA, 20 bp, Homo sapiens
aaacagggggaaaatatggg

SEQ ID NO: 517 - DNA, 20 bp, Homo sapiens
ttcgttctgctgaccaaatg

SEQ ID NO: 518 - DNA, 20 bp, Homo sapiens
aaacaaccggtaaacctcca

SEQ ID NO: 519 - DNA, 21 bp, Homo sapiens
gttactgggactgtagcgtct

SEQ ID NO: 520 - DNA, 20 bp, Homo sapiens
ttccgcacatcgcacatcat

SEQ ID NO: 521 - DNA, 22 bp, Homo sapiens
ttaaaggctttctcatgaggct

SEQ ID NO: 522 - DNA, 20 bp, Homo sapiens
aagagcgaatccattccaga

SEQ ID NO: 523 - DNA, 25 bp, Homo sapiens
aaatggtctagaagttttcgtttcc

SEQ ID NO: 524 - DNA, 17 bp, Homo sapiens
aaaaagcgagtggcgga

SEQ ID NO: 525 - DNA, 20 bp, Homo sapiens
taccgacgcagacgcccaga

SEQ ID NO: 526 - DNA, 20 bp, Homo sapiens
agcctcgggagcggacgaat

SEQ ID NO: 527 - DNA, 20 bp, Homo sapiens
gccagccccggtctctttcc

SEQ ID NO: 528 - DNA, 20 bp, Homo sapiens
cgccggagtcaggcccaaat

SEQ ID NO: 529 - DNA, 20 bp, Homo sapiens
tcaccgccgtgagcccaagt

SEQ ID NO: 530 - DNA, 20 bp, Homo sapiens
tcgccgctcccctcagagac

SEQ ID NO: 531 - DNA, 20 bp, Homo sapiens
taaaacccttcccctgtcca

SEQ ID NO: 532 - DNA, 20 bp, Homo sapiens
ttagctattcaaggtggggg

SEQ ID NO: 533 - DNA, 26 bp, Homo sapiens
ccccaaatagtagatgtctaaaatca

SEQ ID NO: 534 - DNA, 27 bp, Homo sapiens
acaatggaatataaaagaatcagatgt